(19) United States
(12) Patent Application Publication
(10) Pub. No.: US 2026/0152798 A1
(43) Pub. Date: Jun. 4, 2026

(54) METHODS FOR DETECTING CIRCULATING GENETICALLY ABNORMAL CELLS

(71) Applicant: LungLife AI, Inc., Thousand Oaks, CA (US)

(72) Inventors: Shahram TAHVILIAN, Tarzana, CA (US); Daniel GRAMAJO-LEVENTON, Northridge, CA (US); Benjamin KATCHMAN, Simi Valley, CA (US); Paul PAGANO, Moorpark, CA (US)

(21) Appl. No.: 18/868,588

(22) PCT Filed: May 24, 2023
(86) PCT No.: PCT/US2023/067430

§ 371 Date: Nov. 22, 2024

Related U.S. Application Data
(60) Provisional application No. 63/345,078, filed on May 24, 2022.

(51) Int. Cl. C12Q 1/6886; C12Q 1/6804

(52) U.S. Cl. CPC C12Q 1/6886; C12Q 1/6804; C12Q 2600/156

(57) ABSTRACT

The present disclosure provides a method of identifying lung cancer in a subject in need there of comprising detecting circulating genetically abnormal cells (CGAC) in a sample comprising a population of cells. CGACs can be identified and classified according chromosomal hybridization patterns detected via fluorescent in situ hybridization (FISH). The identification of lung cancer can also comprise the evaluation of at least one nuclear parameter in the CGAC and/or identifying the presence or absence of an intracellular or cell surface protein in the CGAC.

### RELATED APPLICATIONS

This application is a U.S. National Phase Application, filed under 35 U.S.C. § 371(c), of International Application No PCT/US2023/067430, filed May 24, 2023, which claims priority to, and the benefit of U.S. Provisional Application No. 63/345,078, filed May 24, 2022, the contents of which is hereby incorporated by reference in its entirety.

### BACKGROUND OF THE INVENTION

In 2021, the estimated number of new lung cancer cases exceeded 235,000 (Siegel R L et al. 2021). Identification of lung cancer at earlier stages results in more favorable prognoses and outcomes (Knight S B et al, 2017). Early-stage lung cancer continues to be challenging to detect due to low adherence to lung cancer screening guidelines and because early-stage disease is typically asymptomatic (Siegel R L et al. 2021). Therefore, many lung cancer cases are caught in the later stages of disease when metastasis has occurred and survival is low.

The majority of early-stage lung cancers are initially identified as indeterminate pulmonary nodules (IPNs), and an estimated 1.5 million IPNs are identified in the US each year using CT (Gould M K et al. 2015). Guidelines, such as those from the American College of Chest Physicians (ACCP) and Flieshner Society, are generally in alignment for low or high-risk nodules, where the pre-test probability for lung cancer is <5% or >65%, respectively (Mazzone P J et al. 2021, Bueno J et al. 2018). However, for intermediate risk IPNs (5%-65% pre-test probability for lung cancer), guidelines are poorly aligned and these nodules represent the most challenging to evaluate (Paez R et al. 2021). It is estimated that >40% of biopsies of CT-identified IPNs are negative for lung cancer (Lokhandwala T et al. 2017). As a result, many individuals are unnecessarily exposed to invasive diagnostic procedures, such as biopsy, where approximately 20% of patients experience adverse events, including infection, pneumothorax, hemorrhage and even death from the procedure (Lokhandwala T et al. 2017, Huo J et al. 2019, Handy J R et al. 2020). Altogether, this highlights the need for an improved noninvasive evaluation method that can provide additional information with higher confidence for individuals with IPNs.

Diagnosing cancer using blood, which can be obtained inexpensively and less invasively than a tissue biopsy, is an emerging approach. A main advantage of using blood compared with traditional biopsy is that the specimen is not restricted to a single tumor site. This allows for more diverse sampling of circulating components from the tumor, such as circulating tumor cells (CTCs) and circulating tumor DNA (ctDNA), as well as other circulating components in the blood that may have interacted with the tumor, such as immune cells.

Though metastasis is associated with later-stage disease in many cancer types, very early appearance of metastatic behavior in lung cancer model systems and clinical lung cancers (Pagano P C, et al, 2017 and Tanaka F et al, 2009) has been demonstrated. Previous studies demonstrate that CTCs can be identified in patients with stage I lung cancer (Tanaka F et al, 2009, Chemi F et al, 2019) and those with chronic obstructive pulmonary disease (COPD) at high-risk for lung cancer, even years before frank malignancy is observed radiographically (Ilie M et al, 2014). This early metastatic behavior can be leveraged for early detection of lung cancer using various assays.

Numerous blood-based technologies have emerged to facilitate early detection of lung cancer and these technologies typically measure and assess RNA, proteins, circulating cell-free DNA (cfDNA), ctDNA, or CTC (Seijo L M et al, 2019, Freitas C et al, 2021). However, these technologies are limited by their reliance on broad detection of molecular pathophysiological changes typically associated with high tumor burden in later-stage disease and are less likely to accurately detect early-stage lung cancer. Detection of ctDNA has had a marked impact on treatment stratification for late-stage lung cancer and this approach is being reevaluated for early-stage lung cancer detection. However, it has been described that low tumor cell burden limits the capacity to detect the smallest stage I lung cancers using existing ctDNA profiling approaches (Abbosh et al, 2018). Additionally, detecting early-stage lung cancer based on the presence of CTCs has been challenging given that CTCs are generally only detected in small numbers and in approximately 30% of patients with non-small cell lung cancer (NSCLC) (de Wit S et al, 2015, de Wit S et al, 2019 and Tamminga M et al, 2020). Traditional CTC-based assays also depend on the presence of epithelial markers to isolate and/or identify CTCs. However, metastatic cancer cells commonly undergo epithelial to mesenchymal transition; therefore, identification of these CTCs would be missed with the traditional approaches (Chaffer C L et al, 2011 and Katz R L et al, 2010). Due to the low sensitivity levels and suboptimal performance of these emerging technologies, they continue to be further optimized for clinical use (Seijo L M et al, 2019 and Klein E A et al, 2021).

Chromosomal instability, a hallmark of cancer, can result in genomic copy number variations (CNVs) that can be readily detected with well-established technologies in individual cells. Katz et al describe a method using fluorescence in situ hybridization (FISH), which is employed by the LungLB™ test described herein, to detect CNVs in circulating genetically abnormal cells (CGACs) enriched from the peripheral blood of individuals with indeterminate pulmonary nodules (Katz R L et al, 2010). Studies have reported the presence of CGACs in individuals with various cancers, including lung cancer, and some of these CGACs have been identified as CTCs (Katz R L et al, 2010, Feng M et al, 2020, Katz R L et al, 2020). Additionally, individuals with cancer, including lung cancer, have been reported to have circulating lymphocytes with cytogenetic abnormalities that are identical to those found in cancerous cells from the primary tumor (Rossner P et al, 2005, Wang H et al, 2017, Dave B J et al, 1995, Zhu Y et al, 2002, Katz R L et al, 2010). As FISH is generally a highly specific assay for detecting chromosomal instability that is frequently observed in cancer, this method allows more comprehensive detection of multiple types of CGACs that have been reported to be associated with lung cancer (Katz R L et al, 2010, Ye M et al, 2021).

The disclosure provides, a liquid biopsy assay that utilizes FISH, thus circumventing the cell marker restrictions of traditional CTC-based assays, to detect CGACs in individuals with IPNs.

### SUMMARY

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step (a) performing at least one of: (i) determining the presence or absence of at least one intracellular or cell surface protein; (ii) determining at least one nuclear parameter selected from nuclear area, nuclear roundness, nuclear diameter, or a combination thereof, and (d) determining a CGAC score for each CGAC based on the chromosomal hybridization pattern and at least one of: (i) the presence or absence of the intracellular or cell surface protein; or (ii) the at least one nuclear parameter; (e) determining a sample score by taking the sum of the CGAC scores for all CGAC identified in the biological sample obtained from the subject; and (f) identifying lung cancer in the subject when the sample score is above a predetermined cutoff value.

In some aspects, the fluorescently labeled nucleic acid probes comprise four pluralities of nucleic acid probes, wherein each plurality of nucleic acid probes hybridizes to a distinct chromosomal sequence and comprises a distinct fluorescent label.

In some aspects, a first plurality of fluorescently labeled nucleic acid probes hybridizes to 3p22.1, a second plurality of fluorescently labeled nucleic acid probes hybridizes to 10q22.3, a third plurality of fluorescently labeled nucleic acid probes hybridizes to chromosome 10 centromeric (cep10), and a fourth plurality of fluorescently labeled nucleic acid probes hybridizes to 3q29.

In some aspects, a CGAC does not comprise a chromosomal hybridization pattern consisting of two copies of a first chromosomal sequence, two copies of a second chromosomal sequence, two copies of a third chromosomal sequence, and two copies of a fourth chromosomal sequence.

In some aspects, a cell having a hybridization pattern consisting of two copies of a first chromosomal sequence, two copies of a second chromosomal sequence, two copies of a third chromosomal sequence, and two copies of a fourth chromosomal sequence is defined as a healthy cell.

In some aspects, a CGAC comprises a chromosomal hybridization pattern that comprises a gain of at least two copies of a chromosomal sequence.

In some aspects, a CGAC comprises a chromosomal hybridization pattern that comprises a gain of at least two copies of a chromosomal sequence and a loss of a least one copy of a chromosomal sequence.

In some aspects, a CGAC comprises a chromosomal hybridization pattern that comprises: a gain of at least one copy of a first chromosomal sequence, a gain of at least one copy of a second chromosomal sequence, a loss of at least one copy of a third chromosomal sequence, and a loss of at least one copy of a fourth chromosomal sequence.

In some aspects, a CGAC comprises a chromosomal hybridization pattern that comprises five or more copies of a first chromosomal sequence and five or more copies of a second chromosomal sequence.

In some aspects, a CGAC comprises a chromosomal hybridization pattern that comprises: at least four copies of a first chromosomal sequence, at least four copies of a second chromosomal sequence, two copies of a third chromosomal sequence, two copies of a fourth chromosomal sequence.

In some aspects, a CGAC comprises: at least three copies of a first chromosomal sequence, at least three copies of a second chromosomal sequence, at least three copies of a third chromosomal sequence, and at least three copies of a fourth chromosomal sequence.

In some aspects, the CGAC is an advanced CGAC.

In some aspects, the CGAC score comprises a copy number variation (CNV) score assigned based on the chromosomal hybridization pattern.

In some aspects, the CNV score for a healthy cell is zero.

In some aspects, the copy number variation score is increased by a value of 0.5 for each gain of a chromosomal sequence.

In some aspects, the copy number variation score is increased by a value of 0.5 for each loss of a chromosomal sequence.

In some aspects, the CGAC score is determined by scaling the CNV score according to at least one of: (i) to the presence or absence of the intracellular or cell surface protein; or (ii) the at least one nuclear parameter, to determine the CGAC score.

In some aspects, the nuclear area for a healthy cell identified in the biological sample is assigned a normalized area of about 1.0.

In some aspects, a normalized nuclear area for a CGAC is expressed as a value relative to the nuclear area of the healthy cell.

In some aspects, the nuclear area for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% larger than a healthy cell.

In some aspects, the nuclear area for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% smaller than a healthy cell.

In some aspects, the CGAC normalized area is multiplied by the CNV score to determine the CGAC score for a CGAC of the biological sample.

In some aspects, the nuclear roundness for a healthy cell is assigned a normalized roundness of about 1.0.

In some aspects, a normalized nuclear roundness for a CGAC is expressed as a value relative to the normalized roundness of a healthy cell.

In some aspects, the nuclear roundness for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% more round than a healthy cell.

In some aspects, the nuclear roundness for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% less round than a healthy cell.

In some aspects, the CGAC normalized roundness is multiplied by the CNV score to determine the CGAC score for a CGAC of the biological sample.

In some aspects, nuclear roundness is calculated according to the equation: r=4π(A/p²), wherein r=roundness; A=nuclear area; and p=nuclear perimeter.

In some aspects, a nuclear diameter for a healthy cell is assigned a normalized nuclear diameter of about 1.0.

In some aspects, a normalized nuclear diameter for a CGAC is expressed as a value relative to the normalized diameter of a healthy cell.

In some aspects, the nuclear diameter for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% larger than a healthy cell.

In some aspects, the nuclear diameter for a CGAC can be about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% smaller than a healthy cell.

In some aspects, the CGAC normalized diameter is multiplied by the CNV score to determine the CGAC score for a CGAC of the biological sample.

In some aspects, the CNV score can be multiplied by at least one of the CGAC normalized diameter, the CGAC normalized roundness, and the CGAC normalized area, or a combination thereof, to determine the CGAC score for a CGAC of the biological sample.

In some aspects, the nuclear area, nuclear roundness, or nuclear diameter is determined by flow cytometry, light microscopy, or computer-driven size analysis.

In some aspects, the determining the presence of the at least one intracellular or cell surface protein is determined by the use of an immunofluorescent stain.

In some aspects, the at least one intracellular or cell surface protein is selected from CD45, CD19, CD31, PAX5, AID, BCL6, EGFR, CD3, CD20, IgM, IgD, CD56, EpCAM, Vimentin, FoxP3, KI-67, or a combination thereof.

In some aspects, a CGAC is assigned a biomarker score based on the presence or absence of the at least one intracellular or cell surface protein.

In some aspects, the biomarker score is multiplied by the CNV score to determine the CGAC score for a CGAC of the biological sample.

In some aspects, the CNV score can be multiplied by at least one of the CGAC normalized diameter, the CGAC normalized roundness, the CGAC normalized area, and the biomarker score or a combination thereof, to determine the CGAC score for a CGAC of the biological sample.

In some aspects, the population of cells of the biological sample comprises about 5,000 cells, about 6,000 cells, about 7,000 cells, about 8,000 cells, about 9,000 cells, about 10,000 cells, about 11,000 cells, about 12,000 cells, about 13,000 cells, about 14,000 cells, about 15,000 cells, about 16,000 cells, about 17,000 cells, about 18,000 cells, about 19,000 cells, about 20,000 cells, about 50,000 cells, or about 100,000 cells,

In some aspects, the population of cells of the biological sample comprises about 10,000 cells.

In some aspects, the CGAC score is expressed in a unit of cells.

In some aspects, the sample score is expressed as the sum of all CGAC scores/number of cells of the biological sample.

In some aspects, the sample score predetermined cutoff value is about 0.5 CGAC/10,000 cells, about 1.0 CGAC/10,000 cells, about 1.5 CGAC/10,000 cells, about 2.0 CGAC/10,000 cells, about 2.5 CGAC/10,000 cells, about 3.0 CGAC/10,000 cells, about 3.5 CGAC/10,000 cells, about 4.0 CGAC/10,000 cells, about 5.0 CGAC/10,000 cells, about 5.5 CGAC/10,000 cells, about 6.0 CGAC/10,000 cells, about 6.5 CGAC/10,000 cells, about 7.0 CGAC/10,000 cells, about 7.5 CGAC/10,000 cells, about 8.0 CGAC/10,000 cells, about 8.5 CGAC/10,000 cells, about 9.0 CGAC/10,000 cells, about 9.5 CGAC/10,000 cells, about 10.0 CGAC/10,000 cells, about 15.0 CGAC/10,000 cells, about 20.0 CGAC/10,000 cells, about 25.0 CGAC/10,000 cells, about 30.0 CGAC/10,000 cells, about 40.0 CGAC/10,000 cells, about 50.0 CGAC/10,000 cells, about 60.0 CGAC/10,000 cells, about 70.0 CGAC/10,000 cells, about 80.0 CGAC/10,000 cells, about 90.0 CGAC/10,000 cells, about 100 CGAC/10,000 cells, about 200 CGAC/10,000 cells, about 300 CGAC/10,000 cells, about 400 CGAC/10,000 cells, about 500 CGAC/10,000 cells, about 600 CGAC/10,000 cells, about 700 CGAC/10,000 cells, about 800 CGAC/10,000 cells, about 900 CGAC/10,000 cells, about 1,000 CGAC/10,000 cells, or about 2,000 CGAC/10,000 cells.

In some aspects, the biological sample obtained from the subject is a blood sample.

In some aspects, the disclosure further comprises performing a CGAC enrichment step prior to contacting the biological sample obtained from the subject comprising: (i) removing plasma from the sample, (ii) removing erythrocytes from the sample, (iii) contacting the sample with at least one affinity agent that binds a cell surface protein, and (iv) depleting cells from the sample that express the cell surface marker.

In some aspects, a subject identified as having cancer is referred for surgical resection.

In some aspects, the disclosure further comprises administering a therapeutic agent to the subject having been diagnosed with lung cancer.

The disclosure provides a method for identifying a risk of developing lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step (a) performing at least one of: (i) determining the presence or absence of at least one intracellular or cell surface protein; (ii) determining at least one nuclear parameter selected from nuclear area, nuclear roundness, nuclear diameter, or a combination thereof, and (d) determining a CGAC score for each CGAC based on the chromosomal hybridization pattern and at least one of: (i) the presence or absence of the intracellular cell surface protein; or (ii) the at least one nuclear parameter; (e) determining a sample score by taking the sum of the CGAC scores for all CGAC identified in the biological sample obtained from the subject; and (f) identifying a risk for developing lung cancer in the subject when the sample score is above a predetermined cutoff value.

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step determining at least one nuclear parameter selected from nuclear area, nuclear roundness, nuclear diameter, or a combination thereof, and (d) determining a CGAC score for each CGAC based on the chromosomal hybridization pattern and the at least one nuclear parameter; (e) determining a sample score by taking the sum of the CGAC scores for all CGAC identified in the biological sample obtained from the subject; and (f) identifying lung cancer in the subject when the sample score is above a predetermined cutoff value.

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) determining a CGAC score for each CGAC based on the chromosomal hybridization pattern; (d) determining a sample score by taking the sum of the CGAC scores for all CGAC identified in the biological sample obtained from the subject; and (e) identifying lung cancer in the subject when the sample score is above a predetermined cutoff value.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed disclosure. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIG. 1 is a series of images depicting copy number variation observed in a 4-color fluorescence in-situ hybridization of CGACs derived from peripheral blood of patients having indeterminate nodules. Depicted are double deletion CGAC, super CGAC, 4/4/2/2 (4×2) CGAC, and 3/3/3/3 CGAC.

FIG. 2A is an immunofluorescence image of CGACs visualized using DAPI stain. Target cell 1606 is boxed and identified on the image.

FIG. 2B is an immunofluorescence image of CGACs visualized using CD45-FITC stain. Target cell 1606 is boxed and identified on the image as being CD45 negative (no green fluorescence).

FIG. 2C is an image of target cell 1606 following LungLB assay and depicts a pattern of 4R/2Gd/4Gr/2Aq. R=Red; 3p22.1, Gd=gold; 10q22.3, Gr=green; 3q29, and Aq=aqua; 10 centromeric.

FIG. 3A is a series of photos depicting Target cell 4255 stained with DAPI (left image), CD45-FITC (center image), and LungLB assay images (Right images). Target cell 4255 is CD45 negative and identified as 2R/4Gd/2Gr/4Aq. R=Red, Gd=gold, Gr=green, and Aq=aqua.

FIG. 3B is a series of photos depicting Target cell 4259 stained with DAPI (left image), CD45-FITC (center image), and LungLB assay images (Right image). Target cell 4259 is CD45 positive and identified as 3R/2Gd/3Gr/2Aq. R=Red; 3p22.1, Gd=gold; 10q22.3, Gr=green; 3q29, and Aq=aqua; 10 centromeric.

FIG. 4 is a series of graphs depicting CGAC ratio/10,000 cells following LungLB assay. Depicted are 4×2 (FIG. 4A), double deletion (FIG. 4B), 3/3/3/3 (FIG. 4C), and super CGAC (FIG. 4D).

FIG. 5 is an unweighted ROC analysis performed on the 151 subject data set consisting of 112 Malignant and 39 Benign patient samples. Sensitivity (72.32%), Specificity (71.79%), AUC (73.79%).

FIG. 6 is a weighted ROC analysis performed on the 151 subject data set consisting of 112 Malignant and 39 Benign patient samples. Sensitivity (76.79%), Specificity (71.79%), AUC (78.34%).

FIG. 7 is a graph depicting normalized nuclear area of CGAC having the indicated chromosomal probe patterns.

FIG. 8 is a series of graphs depicting normalized nuclear area (FIG. 8A) and nuclear roundness (FIG. 8B) for double deletion CGAC. CGACs were normalized against the mean of all normal cells per individual slide. Therefore 1.0 is equivalent to the mean nuclear area/roundness of genetically normal white blood cells (WBC).

FIG. 9 is a process flow diagram for CGAC-level analysis on probe pattern and morphology.

FIG. 10 is a graph depicting AUC analysis of CGAC FISH data with removal of specific classes of CGAC or probe patterns. Removal of the “Mismatch” CGAC class has a positive impact on AUC compared to baseline. The baseline result (Avg Normalized) had no CGAC removed and showed an AUC of 0.7568, and is denoted by the black dotted line. Red/Green CGAC (R) removed resulted in an expected drop in AUC to 0.6159 (denoted by the red dotted line). AUC results are displayed from left to right in descending order.

FIG. 11 is a graph of benign, malignant, and indeterminate lesions and the percent of each CGAC class comprising each lesion type. Supervised analysis reveals Mismatch CGAC commonly found in subjects with benign lesions and Double Deletion CGAC commonly found in patients with malignancy.

FIG. 12A (FIG. 1a) is a series of images depicting a normal white blood cell (WBC) with a diploid copy number per FISH probe, indicated by 2 spots detected per probe color channel (Red, Green, Aqua, and Gold). A DAPI stained image is also presented along with an overlay of all images.

FIG. 12B (FIG. 1B) is a series of images depicting a representative CGAC with an extra spot in the Red and Green FISH probes. A DAPI stained image is also presented along with an overlay of all images.

FIG. 12C (FIG. 1C) is a series of images depicting an advanced CGAC having 4 spots in 2 color channels and 2 spots in 2 color channels (4×2 Advanced CGAC). A DAPI stained image is also presented along with an overlay of all images.

FIG. 12D (FIG. 1D) is a series of images depicting an advanced CGAC having a gain of two spots in 2 color channels plus any loss of spots in 2 color channels (Double Deletion Advanced CGAC). A DAPI stained image is also presented along with an overlay of all images.

FIG. 13A (FIG. 2A) is a graph depicting an ROC analysis of the full data set consisting of 151 participants revealed an AUC of 0.74 (95% CI, 0.66-0.84; P<0.001), compared with an AUC of 0.52 using the Mayo Clinic Model, and 67.9% sensitivity and 74.4% specificity.

FIG. 13B (FIG. 2B) is a graph depicting an ROC analysis where advanced CGACs were weighted more heavily revealing an AUC of 0.78 (95% CI, 0.70-0.87; P<0.0001), compared with an AUC of 0.52 using the Mayo Clinic Model, and 77% sensitivity and 72%.

FIG. 14 is a graph depicting a Normalized Nuclear Area of CGACs and Advanced CGACs from Benign and Malignant Participant Samples. The normalized nuclear area for CGACs (gray dots, n=1528) and Advanced CGACs (black dots, n=68) is shown. The nuclear areas of CGACs and Advanced CGACs were normalized to the average nuclear area of normal WBCs from each respective participant's sample. Normalized nuclear area values >1 indicate a larger nuclear area compared with normal WBCs; normalized nuclear area values <1 indicate smaller nuclear area compared with normal WBCs. The horizontal black bar represents the median normalized nuclear area, which was 1.03 for CGACs and 1.15 for Advanced CGACs (mean normalized nuclear area was 1.07 for CGACs vs 1.21 for Advanced CGACs).

FIG. 15A is a graph depicting CGAC scores calculated from the copy number variation value for benign and malignant double deletion CGACs.

FIG. 15B is a graph depicting CGAC scores calculated from the copy number variation values for benign and malignant double deletion CGACs when the CGAC score further accounts for the normalized nuclear area of each cell.

FIG. 15C is a graph depicting CGAC scores calculated from the copy number variation values for benign and malignant double deletion CGACs when the CGAC score further accounts for the normalized nuclear roundness of each cell.

FIG. 15D is a graph depicting CGAC scores calculated from the copy number variation values for benign and malignant double deletion CGACs when the CGAC score further accounts for the normalized nuclear area, weighted normalized roundness, and normalized diameter of each cell.

FIG. 16A is a graph depicting copy number variation scores for benign and malignant double deletion CGACs.

FIG. 16B is a graph depicting the normalized nuclear areas for benign and malignant double deletion CGACs.

FIG. 16C is a graph depicting the normalized roundness values for benign and malignant double deletion CGACs.

FIG. 16D is a graph depicting the normalized nuclear diameter values for benign and malignant double deletion CGACs.

FIG. 16E is a graph depicting a CGAC score calculated from the CNV score, normalized nuclear area, and normalized nuclear roundness.

FIG. 16F is a graph depicting a CGAC score calculated from the CNV score, normalized nuclear area, normalized nuclear area and normalized nuclear roundness.

FIG. 17 is a graph depicting CGAC score for benign and malignant advanced CGACs.

FIG. 18 is a series of CGAC depicting CGAC scores (referred to in the photo as LungLB scores). Cells can have very large nuclear areas and large copy number variations resulting in large CGAC scores.

### DETAILED DESCRIPTION

### Methods of the Disclosure

A major goal of cancer diagnostics is the development of improved methods of detection that can accurately identify cancer in a subject in need thereof as early and non-invasively as possible. The present disclosure provides methods of detecting lung cancer in a subject utilizing a fluorescent in situ hybridization-based liquid biopsy by identifying and analyzing circulating genetically abnormal cells (CGAC) isolated from a blood sample obtained from a subject based on the observed chromosomal hybridization pattern as well as additional factors including presence or absence of specific cell biomarkers and changes in nuclear morphology. The combined analysis of the chromosomal hybridization patterns along with these other biomarker or nuclear parameters provide improved sensitivity and ability to detect and/or diagnose lung cancer in a subject.

Circulating genetically abnormal cells are cells having genetic abnormalities and/or chromosomal variation. In some aspects, CGAC arise due to the chromosomal rearrangements and/or aneusomy that occur during cell replication. Such genetic instability is commonly associated with cancers such as lung cancer.

In some aspects, the present disclosure provides methods for identifying a subject at risk for the development of cancer. In some aspects, the present disclosure provides methods for identifying lung cancer in a subject in need thereof. In some aspects, the present disclosure provides methods of detecting cancer in a subject. In some aspects, the subject at risk for the development of lung cancer has one or more indeterminate pulmonary nodules.

The present disclosure provides a method for identifying lung cancer in a subject in need thereof comprising contacting a biological sample obtained from the subject comprising a population of cells with fluorescently labeled nucleic acid probes and performing fluorescence in situ hybridization to determine chromosomal hybridization patterns.

The method can further comprise identifying circulating genetically abnormal cells (CGAC) in the population of cells based on the chromosomal hybridization patterns determined.

For each CGAC identified the method can further comprise performing at least one of: (i) determining the presence or absence of at least one intracellular or cell surface protein; (ii) determining at least one nuclear parameter selected from nuclear area, nuclear roundness, nuclear diameter, or a combination thereof.

The method can further comprise determining a CGAC score for each CGAC based on the chromosomal hybridization pattern and at least one of: (i) the presence or absence of the intracellular or cell surface protein; or (ii) the at least one nuclear parameter.

The method can further comprise determining a sample score by taking the sum of the CGAC scores for all CGAC identified in the biological sample obtained from the subject.

The method can further comprise identifying lung cancer in the subject when the sample score is above a predetermined cutoff value.

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step (a) performing at least one of: (i) determining the presence or absence of at least one intracellular or cell surface protein; (ii) determining at least one nuclear parameter selected from nuclear area, nuclear roundness, nuclear diameter, or a combination thereof, and (d) determining a CGAC score for each CGAC based on the chromosomal hybridization pattern and at least one of: (i) the presence or absence of the intracellular cell surface protein; or (ii) the at least one nuclear parameter; (e) determining a sample score by taking the sum of the CGAC scores for all CGAC identified in the biological sample obtained from the subject; and (f) identifying lung cancer in the subject when the sample score is above a predetermined cutoff value.

The disclosure provides a method for identifying the risk of developing lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step (a) performing at least one of: (i) determining the presence or absence of at least one intracellular or cell surface protein; (ii) determining at least one nuclear parameter selected from nuclear area, nuclear roundness, nuclear diameter, or a combination thereof, and (d) determining a CGAC score for each CGAC based on the chromosomal hybridization pattern and at least one of: (i) the presence or absence of the intracellular cell surface protein; or (ii) the at least one nuclear parameter; (e) determining a sample score by taking the sum of the CGAC scores for all CGAC identified in the biological sample obtained from the subject; and (f) identifying a risk for developing lung cancer in the subject when the sample score is above a predetermined cutoff value.

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) for each CGAC identified in step determining at least one nuclear parameter selected from nuclear area, nuclear roundness, nuclear diameter, or a combination thereof, and (d) determining a CGAC score for each CGAC based on the chromosomal hybridization pattern and the at least one nuclear parameter; (e) determining a sample score by taking the sum of the CGAC scores for all CGAC identified in the biological sample obtained from the subject; and (f) identifying lung cancer in the subject when the sample score is above a predetermined cutoff value.

The disclosure provides a method for identifying lung cancer in a subject in need thereof comprising: (a) contacting a biological sample obtained from the subject comprising a population of cells with fluorescently labeled nucleic acid probes and performing fluorescence in situ hybridization to determine chromosomal hybridization patterns; (b) identifying circulating genetically abnormal cells (CGAC) in the population of cells based on the chromosomal hybridization patterns determined in step (a); (c) determining a CGAC score for each CGAC based on the chromosomal hybridization pattern; (d) determining a sample score by taking the sum of the CGAC scores for all CGAC identified in the biological sample obtained from the subject; and (e) identifying lung cancer in the subject when the sample score is above a predetermined cutoff value.

In some aspects, the subject is identified as having cancer when the sample score for the biological sample is above the predetermined cutoff value.

In some aspects, the subject at risk for the development of cancer is at risk for developing cancers of lung, breast, colon, prostate, pancreas, esophagus, all gastro-intestinal tumors, urogenital tumors, kidney cancers, melanomas, endocrine tumors, sarcomas, etc. In some aspects, the subject at risk for the development of lung cancer.

In some aspects, the test sample comprises blood cells. In some aspects, the test sample comprises saliva, peripheral blood cells, bone marrow, or stem cells isolated from blood or bone marrow. In some aspects, the test sample is peripheral blood.

In some aspects, the peripheral blood is obtained from the subject by a peripheral blood draw.

### Circulating Genetically Abnormal Cell Enrichment

The present disclosure provides an improved and superior method of enriching, isolating, and/or identifying circulating genetically abnormal cells_(CGAC) from a test sample.

In some aspects, the present disclosure provides a method of performing an enrichment step on a whole blood sample comprising a population of cells obtained from a subject comprising: removing plasma from the sample, removing erythrocytes from the sample, contacting the sample with at least one affinity agent that binds a cell surface marker, and depleting cells from the sample that express the cell surface marker. The resulting population of cells is enriched for CGACs.

In some aspects, the CGACs are enriched from a test sample wherein the test sample is whole blood. In some aspects, the sample is fresh blood. In some aspects, the sample is fixed blood. In some aspects, fixed blood is blood that is stabilized using chemicals that cross-link proteins and DNA such that normal clotting and degradation processes are significantly slowed or stopped.

In some aspects, plasma is removed from the sample. In some aspects, plasma is removed from the sample by centrifugation. In some aspects, the sample is centrifuged for at least 1 min, at least 2 min, at least 3 min, at least 4 min, at least 5 min, at least 6 min, at least 7 min, at least 8 min, at least 9 min, at least 10 min, at least 11 min, at least 12 min, at least 13 min, at least 14 min, at least 15 min, or at least 20 min. In some aspects, the sample is centrifuged for 10 min. In some aspects, the sample is centrifuged at 100×g, 200×g, 300× g, 400×g, 500×g, 600×g, 700×g, 800×g, 900×g, or 1000×g. In some aspects, the sample is centrifuged at 700×g.

In some aspects, following centrifugation, the plasma is removed from the sample and stored at −80° C.

In some aspects, removal of neutrophils, monocytes, and granulocytes reduces the rate of false negative samples as analyzed by FISH.

In some aspects, erythrocytes are removed from the sample. In some aspects, erythrocytes are removed by cell lysis. In some aspects, the sample is contacted with an erythrocyte lysis buffer. In some aspects, the erythrocyte lysis buffer is an ammonium chloride lysis buffer. In some aspects, the erythrocyte lysis buffer contains ammonium chloride. In some aspects, the erythrocyte lysis buffer contains sodium bicarbonate. In some aspects, the erythrocyte lysis buffer contains ethylenediaminetetraacetic acid (EDTA). In some aspects, the erythrocyte lysis buffer contains ammonium chloride (8.29 grams), sodium bicarbonate (0.2 grams), Ethylenediaminetetraacetic acid (1.1 grams) and water (90.494 milliliters). In some aspects, the erythrocyte lysis buffer contains ammonium chloride at a concentration of 0.01 M to 5 M, 0.1 M to 4 M, 0.5 M to 3 M, or 1 M to 2 M. In some aspects, the erythrocyte lysis buffer contains ammonium chloride at a concentration of 1.0 M, 1.1 M, 1.2 M, 1.3 M, 1.4 M, 1.5 M, 1.55 M, 1.6 M, 1.7 M, 1.8 M, 1.9 M, or 2 M. In some aspects, the erythrocyte lysis buffer contains sodium bicarbonate at a concentration of 1 mM to 200 mM, 5 mM to 150 mM, 15 mM to 100 mM, or 20 mM to 40 mM. In some aspects, the erythrocyte lysis buffer contains sodium bicarbonate at a concentration of 20 mM, 21 mM, 22 mM, 23 mM, 24 mM, 25 mM, 26 mM, 27 mM, 28 mM, 29 mM, or 30 mM. In some aspects, the erythrocyte lysis buffer contains Ethylenediaminetetraacetic acid at a concentration of 1 mM to 200 mM, 5 mM to 150 mM, 15 mM to 100 mM, or 25 mM to 45 mM. In some aspects, the erythrocyte lysis buffer contains Ethylenediaminetetraacetic acid at a concentration of 30 mM, 31 mM, 32 mM, 33 mM, 34 mM, 35 mM, 36 mM, 37 mM, 37.6 mM, 38 mM, 39 mM, 40 mM, 41 mM, 42 mM, 43 mM, 44 mM, or 45 mM.

In some aspects, the sodium bicarbonate concentration is different for fresh blood and fixed blood samples. In some aspects, different sodium bicarbonate concentrations alter the number of granulocytes that change in size and granularity. In fixed blood, the widely-used bicarbonate concentration results in a left-shift (size reduction) of granulocytes. In some aspects, increased sodium bicarbonate concentration exacerbates the observation. In some aspects, lower sodium bicarbonate concentration rescues the phenotype (granulocytes keep a normal size).

In some aspects, following erythrocyte removal, cells are further removed from the sample using surface marker depletion. In some aspects, the sample is contacted with at least one affinity agent. In some aspects, the affinity agent is an antibody. In some aspects, the affinity agent is a biotinylated affinity agent. In some aspects, the antibody is a dextran-antibody conjugate. In some aspects, the depletion comprises the use of a magnetic particle. In some aspects, the magnetic particle is a strepatavidin coated magnetic particle or an anti-dextran antibody-coated magnetic particle. In some aspects, the biotinylated affinity agent binds a cell surface marker. In some aspects, the cell surface marker is specific for a cell type. In some aspects, the cell type is a neutrophil, monocyte, plasma cell or lymphocyte. In some aspects, the cell type is a neutrophil or monocyte. In some aspects, the lymphocyte is a B-cell and subpopulations thereof, a natural killer (NK) cell and subpopulations thereof, or a T-cell and subpopulations thereof. In some aspects, the B-cell is a naïve B-cell or a mature B-cell. In some aspects, the T-cell is a T-helper cell, a cytotoxic T-cell, or regulatory T-Cells. In some aspects, the cell surface marker is CD66b, CD14, CD3, CD4, CD8, CD17, CD56, CD19, CD20, CD25, IgM, or IgD. In some aspects the cell surface marker is CD66b or CD14. In some aspects, the neutrophil cell surface marker is CD66b. In some aspects, the monocyte cell surface marker is CD14. In some aspects, CD56 is a natural killer cell surface marker. In some aspects, CD19. CD20, IgM, and IgD are B-cell surface markers.

In some aspects, the biotinylated affinity agent is an anti-CD66b antibody. In some aspects, the biotinylated affinity agent is an anti-CD14 antibody. In some aspects, the biotinylated affinity agent is an anti-CD3 antibody. In some aspects, the biotinylated affinity agent is an anti-CD4 antibody. In some aspects, the biotinylated affinity agent is an anti-CD8 antibody. In some aspects, the biotinylated affinity agent is an anti-CD17 antibody. In some aspects, the biotinylated affinity agent is an anti-CD56 antibody. In some aspects, the biotinylated affinity agent is an anti-CD19 antibody. In some aspects, the biotinylated affinity agent is an anti-CD20 antibody. In some aspects, the biotinylated affinity agent is an anti-CD25 antibody. In some aspects, the biotinylated affinity agent is an anti-IgM antibody. In some aspects, the biotinylated affinity agent is an anti-IgD antibody.

In some aspects, combinations of biotinylated affinity agents are used. In some aspects, the sample is contacted with at least two, at least three, at least four, at least five, at least six, at least seven, at least eight, at least nine, or at least ten biotinylated affinity agents. In some aspects, the sample is contacted with at least two biotinylated affinity agents. In some aspects, the sample is contacted with at least three biotinylated affinity agents. In some aspects, the sample is contacted with at least four biotinylated affinity agents. In some aspects, the sample is contacted with at least five biotinylated affinity agents. In some aspects, the sample is contacted with an anti-CD66b antibody and an anti-CD14 antibody. In some aspects, the sample is contacted with an anti-CD66b antibody, an anti-CD14 antibody, and an anti-CD13 antibody. In some aspects, the sample is contacted with an anti-CD66b antibody, an anti-CD14 antibody, an anti-CD13 antibody, and an anti-CD56 antibody. In some aspects, the sample is contacted with an anti-CD66b antibody, an anti-CD14 antibody, an anti-CD13 antibody, and an anti-CD19 antibody. In some aspects, the sample is contacted with an anti-CD66b antibody, an anti-CD14 antibody, an anti-CD13 antibody, an anti-CD56 antibody, and an anti-CD19 antibody.

In some aspects, following contacting the sample with biotinylated affinity agents, the sample is contacted with streptavidin-coated magnetic particles. In some aspects, following incubation with the streptavidin-coated magnetic particles, the sample is exposed to a magnet to magnetically separate the cells expressing the targeted cell surface markers from the sample.

In some aspects, cells are enriched via fluorescent-activated cell sorting (FACS). In some aspects, enrichment via FACS utilizes fluorescent streptavidin antibody conjugates.

### Affinity Agents

In some aspects, the affinity agent is an antibody. In some aspects, the affinity agent is a biotinylated affinity agent. In some aspects, the antibody is a dextran-antibody conjugate. In some aspects, affinity agents of the disclosure are biotinylated affinity agents. In some aspects, streptavidin-coated particles are used to bind biotinylated affinity agents and deplete and or harvest cells bound to the biotinylated affinity agent specific to a particular cell surface marker. In some aspects, affinity agents of the disclosure are directly conjugated to magnetic particles. In some aspects, affinity agents of the disclosure are Anti-Phycoerythrin (PE) MicroBeads. In some aspects, anti-PE microbeads are used for the indirect magnetic labeling and separation of cells with a PE-conjugated primary antibody. In some aspects, affinity agents of the disclosure are digoxigenin (DIG) conjugated antibodies and anti-DIG magnetic beads/particles are used in methods of the disclosure.

### Enrichment of CGACs Via Positive Selection

In some aspects, the enrichment step further comprises: contacting the sample with at least one additional affinity agent that binds a cell surface marker, contacting the sample with particles that binds the affinity agent and collecting cells that express the cell surface marker. In some aspects, the collected cells are then utilized in the FISH assays described herein. In some aspects, the cell surface marker is CD66b, CD14, CD3, CD4, CD8, CD17, CD56, CD19, CD20, CD25, IgM, or IgD. In some aspects, the cell surface marker is a B-cell specific marker that comprises CD19, CD20, IgM, or IgD. In some aspects, the cell surface marker is CD66b, CD14, CD3, CD4, CD8, CD17, CD56, CD19, CD20, CD25, IgM, or IgD. In some aspects, the at least one additional biotinylated affinity agent comprises an anti-CD19 antibody, an anti-CD20 antibody, an anti-IgM antibody, or an anti-IgD antibody. In some aspects, the collected cells comprise lymphocytes. In some aspects, the lymphocytes are B-cells.

In some aspects, the cell surface marker is a B-cell specific cell surface marker. In some aspects, the B-cell specific cell surface marker is CD19. In some aspects, the at least one biotinylated affinity agent comprises an anti-CD19 antibody.

In some aspects, positive and negative selection methods can be combined. For example, cells expressing one or more cell surface markers can be depleted from the sample (negative selection) followed by collection (positive selection) of cells expressing one or more additional surface markers.

### Cell Cryopreservation and Ampule Thawing

In some aspects, blood cells including leukocytes not used in the CGAC enrichment procedure are fixed with a paraformaldehyde solution and washed once with PBS containing 10% FBS. In aspects, the cells are resuspended in 1 mL cryopreservation medium containing 10% DMSO and slowly frozen in a −80° C. freezer (−1° C./min) and then transferred to liquid nitrogen. In some aspects, aliquots of frozen cells are thawed in a 37° C. water bath for approximately 2 minutes, followed by two washes with 10 mL PBS containing 10% FBS to reduce DMSO.

### Fluorescence In-Situ Hybridization (FISH)

The disclosure provides methods of detecting CGAC in a biological sample obtained from a subject comprising the use of FISH. In some aspects, the methods of the disclosure comprise contacting a biological sample obtained from the subject comprising a population of cells with fluorescently labeled nucleic acid probes and performing fluorescence in situ hybridization (FISH) to determine chromosomal hybridization patterns. In some aspects, the nucleic acid probes are specific for any genetic marker that is most frequently amplified or deleted in CGAC. In some aspects, the nucleic acid probes are specific to 3p22.1, 10q22.3, chromosome 10 centromeric (cep10), 3q29 or chromosome 3 centromeric (cep3). In some aspects, the labeled nucleic acid probes for 3p22.1 is an RPL14, CD39L3, PMGM, or GC20 probe. In some aspects, the labeled nucleic acid probes for 10q22.3 is a surfactant protein A1 or surfactant protein A2 probe.

Methods of the disclosure can comprise the use of any number of fluorescently labeled nucleic acid probes. Each fluorescently labeled nucleic acid probe can bind and/or hybridize to a distinct sequence of chromosomal DNA. Further, each fluorescently labeled nucleic acid probe can comprise a distinct fluorescent label. The use of distinct fluorescent labels enables the ability to spectrally distinguish each probe.

In some aspects, at least one, at least two, at least three, at least four, at least five, or at least six distinct fluorescently labeled nucleic acid probes are used.

In a preferred aspect, the fluorescently labeled nucleic acid probes comprise four pluralities of nucleic acid probes, wherein each plurality of nucleic acid probes hybridizes to a distinct chromosomal sequence and comprises a distinct fluorescent label. In some aspects, the four pluralities of nucleic acid probes comprise a first plurality of fluorescently labeled nucleic acid probes, a second plurality of fluorescently labeled nucleic acid probes, a third plurality of fluorescently labeled nucleic acid probes, and a fourth plurality of fluorescently labeled nucleic acid probes.

In some aspects, a first plurality of fluorescently labeled nucleic acid probes hybridizes to 3p22.1, a second plurality of fluorescently labeled nucleic acid probes hybridizes to 10q22.3, a third plurality of fluorescently labeled nucleic acid probes hybridizes to chromosome 10 centromeric (cep10), and a fourth plurality of fluorescently labeled nucleic acid probes hybridizes to 3q29.

The fluorescent labels can be any fluorescent label known in the art. In some aspects, a first fluorescent label is green, a second fluorescent label is red, a third fluorescent label is gold/yellow, and a fourth fluorescent label is aqua/blue. In some aspects, a first plurality of fluorescently labeled nucleic acid probes hybridizes to 3p22.1 and comprises a red fluorescent label, a second plurality of fluorescently labeled nucleic acid probes hybridizes to 10q22.3 and comprises a gold/yellow fluorescent label, a third plurality of fluorescently labeled nucleic acid probes hybridizes to chromosome 10 centromeric (cep10) and comprises an aqua/blue fluorescent label, and a fourth plurality of fluorescently labeled nucleic acid probes hybridizes to 3q29 and comprises a green fluorescent label.

In some aspects, the biological sample comprising a population of cells are fixed with Carnoy's fixative (3:1 solution of methanol and glacial acetic acid) for 30 minutes. In some aspects, the cells are fixed using 95% ethanol. Following cell fixation, the sample is contacted with a protease. In some aspects, the protease is pepsin. Following incubation with a protease, the sample is contacted with labelled nucleic acids.

The population of cells of the biological sample can comprise any number of cells. In some aspects, the population of cells of the biological sample comprises about 5,000 cells, about 6,000 cells, about 7,000 cells, about 8,000 cells, about 9,000 cells, about 10,000 cells, about 11,000 cells, about 12,000 cells, about 13,000 cells, about 14,000 cells, about 15,000 cells, about 16,000 cells, about 17,000 cells, about 18,000 cells, about 19,000 cells, about 20,000 cells, about 50,000 cells, about 100,000 cells, about 500,000 cells, about 1,000,000 cells, or about 10,000,000 cells, (or any number in between). In some aspects of the methods of the disclosure, any number of cells are analyzed can comprise about 5,000 cells, about 6,000 cells, about 7,000 cells, about 8,000 cells, about 9,000 cells, about 10,000 cells, about 11,000 cells, about 12,000 cells, about 13,000 cells, about 14,000 cells, about 15,000 cells, about 16,000 cells, about 17,000 cells, about 18,000 cells, about 19,000 cells, about 20,000 cells, about 50,000 cells, about 100,000 cells, about 500,000 cells, about 1,000,000 cells, or about 10,000,000 cells, (or any number in between).

In some aspects at least about 10,000 cells are analyzed from the biological sample. In some aspects, about 10,000 cells are analyzed from each biological sample. In some aspects, the biological sample is split and run in duplicate or triplicate with each of the split samples comprising at least about 10,000 cells.

### CGAC Identification

Methods of the disclosure further comprise determining the chromosomal hybridization patterns of the cells contacted with the fluorescently labeled nucleic acid probes. In some aspects, CGACs can be identified, and differentiated from genetically normal or healthy cell, according to the hybridization pattern following FISH analysis. In some aspects a genetically normal or healthy cell can be defined as a cell having a chromosomal hybridization pattern consisting of two copies of a first chromosomal sequence, two copies of a second chromosomal sequence, two copies of a third chromosomal sequence, and two copies of a fourth chromosomal sequence. In other words, the genetically normal or healthy cell can be observed to have two copies of each chromosomal sequence targeted by each population of fluorescently labeled nucleic acid probes. In some aspects, a cell is classified as normal if the chromosomal hybridization pattern shows 2 spots of each color indicating two copies of each nucleic acid probe. In some aspects, a deletion is a loss of one or more spots belonging to a nucleic acid probe indicating a deletion of a target genetic sequence. In some aspects, a gain is the appearance of an additional spot belonging to a nucleic acid probe indicating a duplication of a target genetic sequence.

CGAC of the disclosure can be detected when the analyzed cell has a chromosomal hybridization pattern that does not consist of two copies of a first chromosomal sequence, two copies of a second chromosomal sequence, two copies of a third chromosomal sequence, and two copies of a fourth chromosomal sequence. In other words, the CGAC will reflect the deletion, duplication, or mutation of at least one chromosomal sequence bound by a fluorescently labeled nucleic acid probe of the disclosure. This genetic change results in the gain of at least one copy of a chromosomal sequence and/or the loss of at least one copy of a chromosomal sequence. Accordingly, the chromosomal hybridization pattern as measured by FISH reflects the gain or loss of at least one colored spot in a fluorescent image of the cell.

In some aspects, a CGAC is identified when the chromosomal hybridization pattern of the nucleic acid probes depicts a gain of two or more chromosomal sequences in a cell. In some aspects, a CGAC is identified when the chromosomal hybridization pattern of the nucleic acid probes depicts a loss of two or more chromosomal sequences in a cell.

In some aspects, a CGAC is identified when the chromosomal hybridization pattern of the nucleic acid probes depicts a gain of two or more chromosomal sequences in a cell and a loss of two or more chromosomal sequences in a cell.

In some aspects, a CGAC comprises a chromosomal hybridization pattern that comprises: a gain of at least one copy of a first chromosomal sequence, a gain of at least one copy of a second chromosomal sequence, a loss of at least one copy of a third chromosomal sequence, and a loss of at least one copy of a fourth chromosomal sequence. In some aspects, this is referred to as a double deletion CGAC.

In some aspects, a CGAC comprises a chromosomal hybridization pattern that comprises five or more copies of a first chromosomal sequence and five or more copies of a second chromosomal sequence. In some aspects, this is referred to as a super CGAC.

In some aspects, a CGAC is identified when the chromosomal hybridization pattern of the nucleic acid probes depicts a gain of four or more copies of at least two chromosomal sequences in a cell and two copies of two additional chromosomal sequences.

In some aspects, a CGAC comprises a chromosomal hybridization pattern that comprises: at least four copies of a first chromosomal sequence, at least four copies of a second chromosomal sequence, two copies of a third chromosomal sequence, two copies of a fourth chromosomal sequence. In aspects, this is referred to as a 4×2 CGAC.

In some aspects, a CGAC is identified when the chromosomal hybridization pattern of the nucleic acid probes depicts a gain of three or more copies of at least four chromosomal sequences in a cell.

In some aspects, a CGAC comprises: at least three copies of a first chromosomal sequence, at least three copies of a second chromosomal sequence, at least three copies of a third chromosomal sequence, and at least three copies of a fourth chromosomal sequence. In some aspects, this is referred to as a 3/3/3/3 CGAC.

In some aspects of the disclosure, specific hybridization patterns indicate that the analyzed cell is an advanced CGAC. An “Advanced CGAC” of the disclosure is identified when it has a hybridization pattern selected from: double deletion pattern, super CGAC pattern, 4×2 pattern, and/or a 3/3/3/3 pattern. In some aspects, an advanced CGAC has a double deletion pattern or a super CGAC pattern.

The presence of CGACs in the biological sample obtained from the subject can indicate an increased risk for the development of cancer in a subject. The presence of CGACs in the biological sample obtained from the subject can indicate an increased risk for the development of lung cancer in a subject. The presence of CGACs in the biological sample obtained from the subject indicate an increased risk for the development of lung cancer in a subject. The presence of CGACs in the biological sample obtained from the subject can indicate the presence of cancer in a subject. The presence of CGACs in the biological sample obtained from the subject can indicate the presence of lung cancer in a subject.

Advanced CGAC can indicate a greater risk of developing cancer relative to a non-advanced CGAC. Advanced CGAC can indicate a greater risk of a subject having cancer relative to a non-advanced CGAC. Thus, in some aspects, when an advanced CGAC is identified in a biological sample obtained from a subject, the subject is at a higher risk of having cancer or can be assigned a higher likelihood of having cancer relative to a biological sample that does not have an advanced CGAC.

Advanced CGAC can indicate a greater risk of developing lung cancer relative to a non-advanced CGAC. Advanced CGAC can indicate a greater risk of a subject having lung cancer relative to a non-advanced CGAC. Thus, in some aspects, when an advanced CGAC is identified in a biological sample obtained from a subject, the subject is at a higher risk of having lung cancer or can be assigned a higher likelihood of having lung cancer relative to a biological sample that does not have an advanced CGAC.

The presence of advanced CGACs in the biological sample obtained from the subject can indicate an increased risk for the development of cancer in a subject. The presence of advanced CGACs in the biological sample obtained from the subject can indicate an increased risk for the development of lung cancer in a subject. The presence of advanced CGACs in the biological sample obtained from the subject indicate an increased risk for the development of lung cancer in a subject. The presence of advanced CGACs in the biological sample obtained from the subject can indicate the presence of cancer in a subject. The presence of advanced CGACs in the biological sample obtained from the subject can indicate the presence of lung cancer in a subject. The presence of advanced CGACs in the biological sample obtained from the subject can indicate the presence of cancer in a subject.

### Copy Number Variation Score

Methods of the disclosure comprise assigning a “copy number variation (CNV) score” for each CGAC identified based on its chromosomal hybridization pattern. Chromosomal instability, a hallmark of cancer, can result in genomic copy number variations that can be readily detected via FISH. A healthy cell is expected to have two copies of each chromosomal sequence bound/hybridized by the fluorescently labeled nucleic acid probes described herein.

Previously known methods of identifying cancer based on the detection of CGACs or circulating tumor cells (CTC) known in the art treat detected genetically abnormal cell as the same, assigning equal weight to each identified CGAC or CTC regardless of the chromosomal hybridization pattern. Thus, the overall risk of having cancer or developing cancer is determined based solely on the number of CGAC or CTC identified in a biological sample obtained from a subject.

Methods described herein assign higher value to CGACs having a greater number of copy number variations and thus are assigned a higher CNV score. In other words, certain CGAC having specific chromosomal hybridization patterns are assigned more value and count as more cells in the final sample score for a given biological sample obtained from a subject because those CGAC having a greater number of copy number variations are associated with a greater likelihood of cancer being present in a subject or that cancer will develop in a subject.

Accordingly, for a healthy cell having a 2/2/2/2 hybridization pattern, a CNV score of 0 is assigned. A CNV score of zero is indicative of a genetically normal cell and thus does not indicate cancer or an increased risk of developing cancer in a subject. As such, a healthy cell adds no value to the overall sample score as the presence of a healthy cell is not predictive of a cancer being present in a subject.

The CNV score quantifies the amount of copy number variability in a CGAC relative to a healthy cell. Accordingly, as more copy number variations are identified based on the chromosomal hybridization pattern of a cell, the CNV score increases.

According to methods described herein, a value of any magnitude can be assigned to a copy number variation. A CNV score can be expressed as whole integers or decimal numbers. Further, a scaling or multiplying factor can be applied to the CNV score.

Methods of the disclosure assign a copy number variation score of 0.5 for each copy number variation detected in a cell. Thus, for a representative CGAC having a gain of two chromosomal regions the CNV score would be 1.0. For a representative CGAC having a gain of two chromosomal regions and the loss of two chromosomal regions the CNV score would be 2.0.

The CNV score for representative CGACs is outlined in Table A.

TABLE A
CNV scores for representative CGAC types

| CGAC type | Probe pattern | CNV Score |
|---|---|---|
| Healthy cell | 2/2/2/2 | 0 |
| (not a CGAC) | | |
| 3/3/2/2 | 3/3/2/2 | 1 |
| 3 red/3 green | 3/3/2/2 | 1 |
| 2 aqua/2 gold - | | |
| or 3 aqua/3 | | |
| yellow/2 red/ | | |
| 2 green | | |
| 3/3/3/3 | 3/3/3/3 | 2 |
| 4/3/2/2 | 4/3/2/2 | 1.5 |
| Super CGAC | 5/5/2/2 | 3+ |
| Super CGAC | 5/5/3/3 | 4 |
| Double deletion | 4/4/0/0 | 4 |
| 4 × 2 | 4/4/2/2 | 2 |

As depicted in Table A, a healthy cell, which is not a CGAC, has a CNV score of 0 because it has a normal chromosomal hybridization pattern. A 3/3/3/3 CGAC has a CNV score of 2 with a CNV value of 0.5 assigned for each additional chromosome region identified in each probe channel. A super CGAC has a CNV score of 3 because of the gain of three chromosomal sequences in a first channel equates to a CNV value of 1.5 and the gain of three chromosomal sequences in a second channel equates to a CNV value of 1.5 for a total CNV score of 3. A double deletion CGAC is assigned a CNV score of 4 because it gains two chromosomal sequences in two channels (CNV value of 2) and loses two chromosomal sequences (CNV value of 2). A 4×2 CGAC is assigned a CNV score of 2 because of the gain of two chromosomal sequences in two channels.

The CNV scores assigned for the specific CGAC in Table A are non-limiting. All CGAC having unique hybridization patterns can be assigned a CNV score.

CNV scores of the disclosure are scaled according to the presence or absence of at least one intracellular or cell surface protein and/or at least one nuclear parameter. The scaled CNV is the CGAC score for a CGAC.

### Nuclear Parameters

The methods of the disclosure provide methods of evaluating at least one nuclear parameter of a cell. The methods of the disclosure provide methods of sorting and/or classifying cells according to the at least one nuclear parameter. In some aspects, the nuclear parameter is selected from nuclear area, nuclear diameter, and/or nuclear roundness. Nuclear parameters can be evaluated according to any method known in the art including, but not limited to, immunofluorescence microscopy, light microscopy, flow cytometry, computer-driven size analysis, and/or fluorescence-activated single cell sorting (FACS). In some aspects, the cell is stained with a nuclear stain. In some aspects, the nuclear stain is 4′,6-diamidino-2-phenylindole (DAPI).

CGAC or advanced CGAC identified according to methods described herein can be further evaluated according to at least one nuclear parameter. In some aspects, the at least one nuclear parameter is selected from nuclear area, nuclear roundness, nuclear diameter, or a combination thereof.

The CGAC score for a CGAC identified according to methods of the disclosure reflects the CNV score (measurement of copy number variation) scaled according to the at least one nuclear parameter. Accordingly, the CGAC score can be greater than the CNV score or lower than the CNV score dependent on the nuclear morphology of the CGAC. A greater CGAC score indicates a greater likelihood of cancer associated with that CGAC identified from a subject. As such, the nuclear parameter can further enhance the CGAC score (i.e., be associated with a greater likelihood of cancer) or decrease the CGAC score (i.e., be associated with a lower likelihood of cancer).

The nuclear parameter can be expressed according to a linear relationship and a given nuclear parameter value can be scaled against the CNV score to determine a CGAC score. The nuclear parameter can also be expressed according to a logarithmic or exponential relationship and a given nuclear parameter value can be scaled against the CNV score to determine a CGAC score.

### Nuclear Roundness

In some aspects, the nuclear roundness of a cell is evaluated.

In some aspects, methods of nuclear roundness evaluation may be performed according to any method known in the art, and in some aspects, involve physical sorting, such as by FACS or other nuclei sorting means, by analysis of optical data using a computer-driven size analysis, or by manual interrogation of cell nuclei, such as by using standard light microscopy. Typically, the nuclei are stained in order to permit assessment/sorting, such as with DAPI. In certain aspects, the nuclei will be obtained from cells and sorted on their own. Cells may be lysed using standard cell lysis protocols.

In some aspects, a “normal” or healthy” cell is a cell that has a normal chromosomal hybridization pattern (2 copies of each probe: 2 red, 2 blue/aqua, 2 gold/yellow, and 2 green) as measured by FISH. An average nuclear roundness can be assigned to all normal or healthy cells in the biological sample obtained from the subject. Accordingly, the average nuclear roundness for a healthy cell in the biological cell obtained from the subject can be assigned an area of 1.0 or about 1.0. In some aspects, CGAC can have nuclear roundness greater than 1.0 or smaller than 1.0.

Methods of the disclosure comprise assigning a normalized nuclear roundness for each identified CGAC. The normalized nuclear roundness reflects the difference in roundness between the nucleus of a CGAC relative to the nuclear roundness of healthy cells in the biological sample. In some aspects, the normalized nuclear roundness for the CGAC can be calculated according to the equation:

Normalized CGAC nuclear roundness=CGAC nuclear roundness/nuclear roundness of a healthy cell.

Nuclear roundness can be calculated according to the equation:

$$r = 4 π ( A / {p}^{2} ) ,$$

wherein
r=roundness; A=nuclear area; and p=nuclear perimeter.

Roundness as calculated by the equation above is a unitless measure and reflects a degree of roundness. Nuclear roundness is a measure of how circular and/or spherical a cell nucleus is.

In some aspects, roundness can be measured by any other roundness measure known in the art.

Roundness of the nuclei can be measured by comparing the differential in diameter of the nuclei across two or more points of the cell. For example, roundness can be evaluated by measuring the diameter at the x-axis and y-axis. Further, the analysis can be performed in three-dimensions by measuring at least a third diameter, for example the z-axis, thereby providing a diameter in the x, y, and z planes. A perfectly round nucleus has identical diameters in the x and y axis. A perfectly spherical cell has identical diameters across the x, y, and y axis. In some aspects, cells nuclei are not round and/or spherical. In some aspects, a cell having lower roundness indicates a nucleus that is ovular or ovoid in shape. In some aspects, a cell having lower roundness indicates a nucleus that is irregular in shape. Perfectly round nuclei have a roundness of 1.0. Cell nuclei can be irregular with a non-circular or non-ovular perimeter. Such irregular nuclei can have a larger perimeter relative to their area and thus have a roundness value less than 1.0.

The normalized CGAC nuclear roundness reflects the relative difference in nuclear roundness of a CGAC relative to the nuclear roundness of a healthy cell in the biological sample. By way of non-limiting example, a healthy cell having a perfectly circular nucleus (roundness of 1.0) can have a nuclear diameter of 5 m corresponding to an area of 19.63 m² and a perimeter of 15.71 m. For a CGAC having the same area but a perimeter of 20 m the roundness decreases to 0.61 indicating a less round nucleus.

The nuclear roundness of CGACs can also be expressed wherein the roundness of a healthy cell in the sample can be assigned a value of 1.0, a CGAC having a lower degree of nuclear roundness can be assigned a value of greater than 1.0 (i.e. greater ellipticity), and a CGAC having a higher degree of roundness can be assigned a value less than 1.0 (i.e. increased nuclear roundness relative to a healthy cell in the sample).

All techniques, methods, units, and means for expressing nuclear diameter of a cell are contemplated for use according to the methods of the disclosure.

In some aspects, a CGAC has reduced roundness relative to a normal or healthy cell. In some aspects, an advanced CGAC has reduced roundness relative to a normal or healthy cell. In some aspects, an advanced CGAC has reduced roundness relative to a non-advanced CGAC. In some aspects, an advanced CGAC has increased roundness relative to a non-advanced CGAC. In some aspects, a CGAC has increased roundness relative to a healthy cell. A CGAC or advanced CGAC having reduced roundness can indicate an increased risk and/or higher likelihood that the CGAC or advanced CGAC is associated with a cancer, including lung cancer, present in the subject.

In some aspects, the nuclear roundness for a CGAC can be at least about 1%, about 5%, about 10%, about 15%, about 20%, about 25%, about 30%, about 35%, about 40%, about, 45%, about 50%, about 60%, about 70%, about 80%, about 90%, about 100%, about 150%, or about 200% (or any percentage in between) less round than a healthy cell.

In some aspects, the nuclear roundness for a CGAC can be at least about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% (or any percentage in between) rounder than a healthy cell.

### Nuclear Area

In some aspects, the nuclear area of a cell is evaluated.

In some aspects, these methods of nuclear area evaluation may be performed according to any method known in the art, and in some aspects, involve physical sorting, such as by FACS or other nuclei sorting means, by analysis of optical data using a computer-driven size analysis, or by manual interrogation of cell nuclei, such as by using standard light microscopy. Typically, the nuclei are stained in order to permit assessment/sorting, such as with DAPI. In certain aspects, the nuclei will be obtained from cells and sorted on their own. Cells may be lysed using standard cell lysis protocols.

In some aspects, a “normal” or healthy” cell is a cell that has a normal chromosomal hybridization pattern (2 copies of each probe: 2 red, 2 blue/aqua, 2 gold/yellow, and 2 green) as measured by FISH. An average nuclear area can be assigned to all normal or healthy cells in the biological sample obtained from the subject. Accordingly, the average nuclear area for a healthy cell in the biological cell obtained from the subject can be assigned a nuclear area of 1.0 or about 1.0. In some aspects, CGAC can have nuclear areas greater than 1.0 or smaller than 1.0.

Methods of the disclosure comprise assigning a normalized nuclear area for identified CGAC. The normalized area reflects the size difference between the nucleus of a CGAC relative to the nuclear area of healthy cells in the biological sample. As such, the normalized nuclear area for the CGAC can be calculated according to the equation:

Normalized CGAC nuclear area=CGAC nuclear area/nuclear area of a healthy cell.

As such, the normalized CGAC nuclear area reflects the relative size of the nucleus of a CGAC to a healthy cell in the biological sample. By way of non-limiting example, a healthy cell can have an average nuclear area in a biological sample of 500 pixels and a CGAC can have a nuclear area of 1000 pixels (as measured by a fluorescent microscope). As such, the normalized CGAC nuclear area would be 2.0 because the CGAC nucleus is twice as large as the healthy cell nucleus. By way of an additional non-limiting example, a healthy cell can have an average nuclear area in a biological sample of 500 pixels and a CGAC can have a nuclear area of 400 pixels (as measured by a fluorescent microscope). As such, the normalized CGAC nuclear area would be 0.8 because the CGAC nucleus is smaller than the healthy cell nucleus.

All techniques, methods, units, and means for expressing nuclear area of a cell are contemplated for use according to the methods of the disclosure.

In some aspects, a CGAC can have a greater nuclear area relative to a normal or healthy cell. In some aspects, an advanced CGAC can have a greater nuclear area relative to a normal or healthy cell. In some aspects, an advanced CGAC can have a greater nuclear area relative to a non-advanced CGAC. In some aspects, an advanced CGAC can have a smaller nuclear area relative to a non-advanced CGAC. In some aspects, a CGAC can have a smaller nuclear area relative to a healthy cell. A CGAC or advanced CGAC having a larger nuclear area can indicate an increased risk and/or higher likelihood that the CGAC or advanced CGAC is associated with a cancer, including lung cancer, present in the subject.

In some aspects, the nuclear area for a CGAC can be at least about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% (or any percentage in between) larger than a healthy cell.

In some aspects, the nuclear area for a CGAC can be at least about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% (or any percentage in between) smaller than a healthy cell.

### Nuclear Diameter

In some aspects, nuclear diameter is evaluated. In some aspects, the nuclear diameter of a given cell can be the average diameter of the cell nucleus taken from at least two distinct measurements of the nuclear diameter. In some aspects, the average diameter of the cell nucleus can be taken from at least two, at least three, at least four, at least five, at least six, at least seven, at least eight, at least nine, at least ten distinct measurements of the nuclear diameter.

In some aspects, these methods of nuclear diameter evaluation may be performed according to any method known in the art, and in some aspects, involve physical sorting, such as by FACS or other nuclei sorting means, by analysis of optical data using a computer-driven size analysis, or by manual interrogation of cell nuclei, such as by using standard light microscopy. Typically, the nuclei are stained in order to permit assessment/sorting, such as with DAPI. In certain aspects, the nuclei will be obtained from cells and sorted on their own. Cells may be lysed using standard cell lysis protocols.

In some aspects, a “normal” or healthy” cell is a cell that has a normal chromosomal hybridization pattern (2 copies of each probe: 2 red, 2 blue/aqua, 2 gold/yellow, and 2 green) as measured by FISH. Accordingly, an average nuclear diameter can be assigned to a normal or healthy cell. An average nuclear diameter can be assigned to all normal or healthy cells in the biological sample obtained from the subject. In some aspects, the average nuclear diameter for a healthy cell can be assigned an area of 1.0 or about 1.0. In some aspects, CGAC can have nuclear diameters greater than 1.0.

Methods of the disclosure comprise assigning a normalized nuclear diameter for identified CGAC. The normalized nuclear diameter reflects the difference in diameter between the nucleus of a CGAC relative to the nuclear diameter of healthy cells in the biological sample. As such, the normalized nuclear diameter for the CGAC can be calculated according to the equation:

$$Normalized CGAC nuclear diameter = CGAC nuclear diameter / nuclear diameter of a healthy cell .$$

As such, the normalized CGAC nuclear diameter reflects the relative difference in size of the nuclear diameter of a CGAC relative to the diameter of a healthy cell in the biological sample. By way of non-limiting example, a healthy cell can have an average nuclear diameter in a biological sample of 100 pixels and a CGAC can have a nuclear diameter of 200 pixels (as measured by a fluorescent microscope). As such, the normalized CGAC nuclear diameter would be 2.0 because the CGAC nuclear diameter is twice as large as the healthy cell nuclear diameter. By way of an additional non-limiting example, a healthy cell can have an average nuclear diameter in a biological sample of 100 pixels and a CGAC can have a nuclear diameter of 80 pixels (as measured by a fluorescent microscope). As such, the normalized CGAC nuclear diameter would be 0.8 because the CGAC nuclear diameter is smaller than the healthy cell nuclear diameter.

All techniques, methods, units, and means for expressing nuclear diameter of a cell are contemplated for use according to the methods of the disclosure.

In some aspects, a CGAC can have a greater nuclear diameter relative to a normal or healthy cell. In some aspects, an advanced CGAC can have a greater nuclear diameter relative to a normal or healthy cell. In some aspects, an advanced CGAC can have a greater nuclear diameter relative to a non-advanced CGAC. In some aspects, an advanced CGAC can have a smaller nuclear diameter relative to a non-advanced CGAC. In some aspects, a CGAC can have a smaller nuclear diameter relative to a healthy cell. A CGAC or advanced CGAC having a larger nuclear diameter can indicate an increased risk and/or higher likelihood that the CGAC or advanced CGAC is associated with a cancer, including lung cancer, present in the subject.

In some aspects, the nuclear area for a CGAC can be at least about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% (or any percentage in between) larger than a healthy cell.

In some aspects, the nuclear area for a CGAC can be at least about 1%, about 5%, about 10%, about 15%, about 20%, about 25%, about 30%, about 35%, about 40%, about 45%, about 50%, about 60%, about 70%, about 80%, about 90%, about 100%, about 150%, or about 200% (or any percentage in between) smaller than a healthy cel

### Cell Roundness

In some aspects, the roundness of a cell can be expressed as a circularity factor (CF). The circularity factor is calculated by modifying the elongation (proportion between the height and the width of the cell) where a perfect circle will have the value of 1. In some aspects, a healthy cell will have a CF of around 1.0 (i.e., a round, circular cell). In some aspects, a CGAC will have a CF greater than 1.0 (i.e., an extended and/or irregularly shaped cell). In some aspects, a CGAC can have a CF much greater than 1.0. In some aspects, a CGAC has a CF of about 1.1, about 1.2, about 1.3, about 1.4, about 1.5, about 1.6, about 1.7, about 1.8, about 1.9, about 2.0, about 3.0, about 4.0, about 5.0, about 6.0, about 7.0, a bout 8.0, about 9.0, or about 10.0, or any number in between.

Cell roundness can be normalized for a given sample. For example, the roundness can be normalized wherein the average CF for the sample can be assigned an average value such as 1.0.

### Nuclear Parameter Evaluation

### Bioview System and Software

The Bioview Duet™ (Rehovot, Israel) system uses a color or monochromatic CCD cameras normally images and classifies all nucleated cells presented on the cytopreparation. The number of cells classified is preset by the operator however usually several thousand cells are scanned. There is a “research” mode or an open software system, that then records for each cell:
1) nuclear area in pixels, based on the DAPI stain, expressed as arbitrary units, thus, if 5000 it means that the cell area is 5000 pixels;
2) nuclear diameter; and
3) nuclear roundness as described above or circularity factor (CFs), calculated by modifying the elongation (proportion between the height and the width of the cell) where a perfect circle will have the value of 1 (lymphocytes have CFs close to 1, abnormal cells have CFs much >>1, due to their nuclear perimeter irregularity).

The nuclear area for the abnormal cells (CGACs) cells was based on the number of pixels occupied by the nucleus as measured on the DAPI stain (a nuclear stain) and can be expressed as arbitrary units. Immunofluorescence staining

### Intracellular and Cell Surface Protein Detection

The methods of the disclosure provide methods of evaluating the presence of absence of at least one intracellular or cell surface protein in CGACs identified according to methods of the disclosure. The methods of the disclosure provide methods of sorting and/or classifying cells according to the presence of absence of at least one intracellular or cell surface protein. Cellular and intracellular proteins can be identified according to any method known in the art including, but not limited to, immunofluorescence microscopy, light microscopy, flow cytometry, and/or fluorescence-activated single cell sorting (FACS). In some aspects, the biological sample is contacted with an antibody specific for the at least one intracellular or cell surface protein. The antibody can be directly conjugated to a detectable label such as a fluorescent dye. In some aspects, the antibody is detected via the use of a secondary detection antibody.

CGAC or advanced CGAC identified according to methods described herein can be further evaluated according to the presence or absence of the intracellular or cell surface protein. In some aspects, the presence or absence of the protein identifies the cell type of the CGAC.

In some aspects, immunofluorescent (IF) stains can be used to detect the at least one intracellular or cell surface protein. IF stains of the disclosure can be any immunofluorescent stain know in the art. In some aspects, IF stains comprise an antibody specific for a specific cellular target that can comprise a cellular protein, nucleic acid, metabolite, or peptide. In some aspects, IF stains can comprise an antibody that binds to an intracellular or cell surface protein. Antibody-based IF stains can be directly conjugated to a fluorescent dye (a primary antibody IF stain) or a secondary antibody comprising a fluorescent dye can be used that binds the primary antibody that binds to the cell surface marker of interest.

The CGAC score for a CGAC identified according to methods of the disclosure reflects the CNV score (measurement of copy number variation) scaled according to the presence or absence of the intracellular or cell surface protein. Accordingly, the CGAC score can be greater than the CNV score or lower than the CNV score dependent on the presence or absence of the intracellular or cell surface protein in the CGAC. A greater CGAC score indicates a greater likelihood of cancer associated with that CGAC identified from a subject. As such, the nuclear parameter can further enhance the CGAC score (i.e., be associated with a greater likelihood of cancer) or decrease the CGAC score (i.e., be associated with a lower likelihood of cancer).

In some aspects, the intracellular or cell surface protein can comprise CD45, CD19, CD31, PAX5, AID, BLC6, EGFR, CD3, CD20, IgM, IgDCD56, EpCAM, or Vimentin, or a combination thereof. In some aspects, the intracellular or cell surface protein can comprise CD45, CD19, or CD31.

Exemplary intracellular and/or cell surface proteins of the disclosure are set forth in Table B.

TABLE B
Exemplary intracellular and cell surface proteins

| Protein | |
|---|---|
| Marker | Protein Description |
| PAX5 | Transcription factor that plays an essential role in commitment |
| | to B-lymphocyte lineage. |
| AID | Involved in somatic hypermutation, and class-switching in B- |
| | lymphocytes |
| BCL6 | Transcriptional repressor mainly required for germinal center |
| | formation and antibody affinity maturation. |
| EGFR | Receptor tyrosine kinase of the ErbB family. |
| CD3 | CD3 is a protein complex and T-cell co-receptor that is involved |
| | in activating both the cytotoxic T cell (CD8) and T helper (CD4) |
| CD56 | CD56 is the archetypal phenotypic marker of natural killer cells. |
| CD31 | Endothelial Cell Marker |
| EpCAM | Epithelial Cell Adhesion Molecule (EpCAM) is a surface marker |
| | on undifferentiated embryonic stem cells and plays functional |
| | roles in proliferation and differentiation. Marker of epithelial |
| | cells. |
| Vimen- | Vimentin are intermediate filaments found in various non- |
| tin | epithelial cells, especially mesenchymal cells. |
| KI-67 | Expression of KI-67 is associated with cell proliferation |
| FoxP3 | FOXP3 protein attaches (binds) to specific regions of DNA and |
| | helps control the activity of genes that are involved in regulating |
| | the immune system. Specifically, lineage specification factor of |
| | Treg cells. |

CD45 can be used as a cell surface marker to differentiate epithelial CGACs (CD45 negative (−)) from white blood cells (CD45 positive (+)).

In some aspects, advanced CGAC can be differentiated from other CGAC based on the presence or absence of the intracellular or cell surface protein. In some aspects, an advanced CGAC is CD45 negative while other CGAC (or common CGACs) are CD45 positive. In some aspects, cells lacking CD45 expression are indicative of an endothelial or epithelial CGAC. In some aspects, endothelial or epithelial CGAC are advanced CGAC. In some aspects, endothelial or epithelial CGAC are correlated with or indicative of a malignant cancer or tumor cell. In some aspects, cells expressing CD45 are a hematopoietic CGAC. In some aspects, CD45 positive CGAC are correlated with or indicative of a benign cell, however, examples exist where CD45 positive CGAC can be indicative of a malignant tumor.

CD31 can be used as a cellular biomarker to identify endothelial cells. In some aspects, a CD31 positive cell is indicative of an endothelial CGAC. In some aspects, an advanced CGAC is CD31 positive.

CD19 can be used as a cellular biomarker to identify B cells. In some aspects, a CD19 positive cell is indicative of B-cell. In some aspects, an advanced CGAC is CD19 positive.

In some aspects, a biomarker score is assigned to CGAC identified according to methods of the disclosure. The biomarker score can be multiplied by the CNV score to determine the CGAC score for the sample.

In some aspects, the biomarker score is a value greater than 1.0 for an intracellular or cell surface protein that is associated with or indicates malignancy or lung cancer in a subject. Examples of cells having a biomarker score greater than 1.0 include, but are not limited to, EGFR, CD31, EpCAM, vimentin, and KI-67.

In some aspects, the biomarker score is a value less than 1.0 for an intracellular or cell surface protein that is associated with a benign cell in a subject. Examples of cells having a biomarker score less than 1.0 include, but are not limited to, CD19, CD45, PAX5, AID, BCL6, CD3, CD56, and FoxP3.

The biomarker score can be expressed according to a linear relationship and a biomarker score value can be scaled against the CNV score to determine a CGAC score. The biomarker score can also be expressed according to a logarithmic or exponential relationship and a given biomarker score value can be scaled against the CNV score to determine a CGAC score.

### Image Acquisition and Analysis

In some aspects, Slides containing cells are imaged using a Bioview Allegro-Plus microscope system (Bioview USA, Billerica, MA). In some aspects, images are acquired using a 60× objective (Olympus, UPlanSapo, 1.35 NA oil immersion) and a FLIR Grasshopper 3 monochrome camera (12-bit, 2448×2048 pixels, 3.4 μm pixel size) controlled using Bioview Duet software. In some aspects, all cells are imaged with 21 transverse sections spanning 0.65 μm.

In some aspects, objects were classified by the Bioview Duet software according to probe copy number variation (“normal” cells show 2 spots of each color, “deletion” is a loss of one or more spots, “single-gain” is an extra spot in one color, and “CGAC” is defined as a gain in two or more channels). CGAC of the disclosure are identified when a cell has a FISH hybridization pattern as described herein. In some aspects, a licensed technician analyzes cells binned in the “CGAC” class by the Bioview Duet software to verify each cell. CGAC counts are normalized by dividing the CTC count by the total number of cells analyzed and multiplying by 10,000. A minimum of 10,000 cells are analyzed per sample obtained from a subject. In some aspects, 25,000 to 30,000 cells are analyzed per sample obtained from a subject. In some aspects, two samples per subject are analyzed. Total CGAC count, total cell count, and normalized CGAC counts were sent for unblinding for each subject CGAC, including advanced CGAC, of the disclosure can be identified via the consideration of multiple cellular parameters such as chromosomal hybridization pattern, nuclear area, nuclear roundness, nuclear diameter and/or immunofluorescent parameter. In some aspects, CGAC having specific combinations of cellular features are more predictive of cancer in a subject.

### CGAC Score Determination

Methods of the disclosure comprise the determination of a CGAC score for each CGAC identified in a biological sample obtained from a subject. The CGAC score for a CGAC identified according to methods of the disclosure reflects the CNV score (measurement of copy number variation) scaled according to the at least one nuclear parameter and/or the presence or absence of at least one intracellular or cell surface protein. Accordingly, the CGAC score can be greater than the CNV score or lower than the CNV score dependent on the nuclear morphology of the CGAC and/or the presence or absence of the at least one intracellular or cell surface protein. A greater CGAC score indicates a greater likelihood of cancer associated with that CGAC from a sample obtained from a subject. As such, the nuclear parameter and/or the presence or absence of at least one intracellular or cell surface protein can further enhance the CGAC score (i.e., be associated with a greater likelihood of cancer) or decrease the CGAC score (i.e., be associated with a lower likelihood of cancer).

The CGAC score can determined by scaling the CNV score according to at least one of: (i) to the presence or absence of the intracellular cell surface protein; or (ii) the at least one nuclear parameter, to determine the CGAC score.

In some aspects, the CGAC score is determined by multiplying the CNV score with the at least one nuclear parameter and or the biomarker score assigned to the CGAC based on the presence of absence of the at least one intracellular or cell surface protein.

In some aspects, the CGAC score is determined by multiplying the CNV score of a CGAC with the nuclear area of the CGAC, the nuclear diameter of the CGAC, the nuclear roundness of the CGAC (or inverse thereof), or a combination thereof.

In some aspects, the CGAC score is determined by multiplying the CNV score of a CGAC with the biomarker score of the CGAC.

In some aspects, the CGAC score is determined by multiplying the CNV score of a CGAC with the nuclear area of the CGAC, the nuclear diameter of the CGAC, the nuclear roundness of the CGAC (or inverse thereof), the biomarker score of the CGAC, or a combination thereof.

In some aspects, the CGAC score is determined by multiplying the CNV score of a CGAC with the nuclear area of the CGAC.

In some aspects, the CGAC score is determined by multiplying the CNV score of a CGAC with the nuclear roundness of the CGAC.

In some aspects, the CGAC score is determined by multiplying the CNV score of a CGAC with the nuclear diameter of the CGAC.

The CNV score can be multiplied by any combination of the nuclear parameters defined herein and or the biomarker score defined herein to arrive at the CGAC score for each CGAC identified in the biological sample obtained from the subject.

The CGAC score can be reflected in the unit of cells. A healthy cell is assigned a CNV score of zero as such, a healthy cell would have a CGAC score of zero reflecting its lack of predictive power at indicating or diagnosing lung cancer is a subject. A CGAC having a CNV score of 1.0 that does not deviate from a healthy cell in any of the nuclear parameters or the biomarker score would be assigned a CGAC score of 1.0. A CGAC having a CNV score of 1.0 and a normalized nuclear area of 1.2 would thus be assigned a CGAC score of 1.2 (1.0 CNV score*1.2 nuclear area) reflecting an increased predictive and/or diagnostic power of identifying lung cancer in a subject. A CGAC having a CNV score of 1.0, a normalized nuclear area of 1.2, and a nuclear diameter of 0.9 would thus be assigned a CGAC score of 1.08 (1.0 CNV score*1.2 nuclear area*0.9 nuclear diameter) reflecting an increased predictive and/or diagnostic power of identifying lung cancer in a subject but slightly lowered by the smaller nuclear diameter.

### Sample Score Determination

The disclosure further provides methods of determining a sample score for the biological sample, wherein the sample score is a measure of the likelihood that cancer is present in a subject when the sample score is above a predetermined cutoff value. The sample score is determined by taking the sum of each CGAC score determined for each CGAC identified in the biological sample. As an example, if 20 CGAC were identified in a sample of 10,000 cells the sample score would be the total sum of the CGAC scores for the 20 identified CGAC. In some aspects, the sample score of 20 is reflected as 20 CGAC. The sample score can be described as number of CGAC/10,000 cells (or any other number of cells) and the sample score for a sample comprising 20 CGAC can be greater than 20 because for each CGAC score the CGAC score can be greater or less than 1.0 based on the CNV score of the cell as well as the impact of the at least one nuclear parameter and/or the presence or absence of the at least one intracellular or cell surface protein.

The sample score can be further modified, adjusted, or scaled according to additional cellular factors and/or patient factors (e.g., age, sex, smoking history, presence of COPD/emphysema, nodule type, location and size, and cancer history).

Cancer Diagnosis and/or Risk Assessment

Methods of the disclosure comprise identifying lung cancer in a subject in need thereof and/or identifying a risk of developing lung cancer in a subject in need thereof comprising determining a sample score for the biological sample obtained from the subject. As discussed herein, the sample score is a total summation of CGAC scores for each CGAC identified in a sample. The CGAC score can be a measure of the CNV score described herein. The CGAC score can be a measure of the CNV score scaled by the at least one nuclear parameter of the CGAC or the presence or absence of the at least one intracellular or cell surface protein of the CGAC.

Methods of the disclosure identify lung cancer in a subject when sample score is above a predetermined cutoff value. In some aspects, the sample score can be expressed as the total number of CGAC identified in a sample. In some aspects, the sample score can be expressed as a number of CGAC per number of cells analyzed. For example, the amount of CGAC per sample can be expressed as the number of CGAC (sum of all CGAC scores for the sample)/10,000 cells. Wherein cells refers to the number of cells analyzed in a biological sample.

Accordingly, when the sample score rises above a predetermined cutoff value, lung cancer can be identified in the subject. As discussed herein the sample score can be described as number of CGAC/10,000 cells and the sample score for a sample comprising 20 CGAC can be greater than 20 because for each CGAC score the CGAC score can be greater or less than one based on the CNV score of the cell as well as the impact of the at least one nuclear parameter and/or the presence or absence of the at least one intracellular or cell surface protein.

In some aspects, when the sample score rises above the predetermined cutoff value, a subject can be classified as at risk for developing lung cancer.

The predetermined cutoff value for the sample score can be adjusted according to a number of factors. These can include, but are not limited to, patient demographics including age, sex, smoking history, presence of COPD/emphysema, nodule type, location and size, and cancer history. These can also include, but are not limited to, the specific chromosomal hybridization pattern analyzed. In some aspects, the total number of CGAC, regardless of chromosomal hybridization pattern, identified per biological sample is used to calculate the sample score. In some aspects, only certain CGAC patterns are counted and those specific CGAC subtypes are used to determine the sample score. For example, only a single CGAC subtype may be evaluated. Alternatively, one or more CGAC subtypes may be evaluated. In some aspects, only advanced CGAC subtypes are quantified. For example, any combination of CGAC selected from one or more of double deletion patterned CGAC, super CGAC patterned CGAC, 4×2 patterned CGAC, and/or a 3/3/3/3 patterned CGAC may be quantified.

In some aspects, the predetermined cutoff value for the sample score is about 0.5 CGAC/10,000 cells, about 1.0 CGAC/10,000 cells, about 1.1 CGAC/10,000 cells, about 1.2 CGAC/10,000 cells, about 1.3 CGAC/10,000 cells, about 1.4 CGAC/10,000 cells, about 1.5 CGAC/10,000 cells, about 1.6 CGAC/10,000 cells, about 1.7 CGAC/10,000 cells, about 1.8 CGAC/10,000 cells, about 1.09 CGAC/10,000 cells, about 2.0 CGAC/10,000 cells, about 2.1 CGAC/10,000 cells, about 2.2 CGAC/10,000 cells, about 2.3 CGAC/10,000 cells, about 2.4 CGAC/10,000 cells, about 2.5 CGAC/10,000 cells, about 2.6 CGAC/10,000 cells, about 2.7 CGAC/10,000 cells, about 2.8 CGAC/10,000 cells, about 2.9 CGAC/10,000 cells, about 3.0 CGAC/10,000 cells, about 3.1 CGAC/10,000 cells, about 3.2 CGAC/10,000 cells, about 3.3 CGAC/10,000 cells, about 3.4 CGAC/10,000 cells, about 3.5 CGAC/10,000 cells, about 3.6 CGAC/10,000 cells, about 3.7 CGAC/10,000 cells, about 3.8 CGAC/10,000 cells, about 3.9 CGAC/10,000 cells, about 4.0 CGAC/10,000 cells, about 4.1 CGAC/10,000 cells, about 4.2 CGAC/10,000 cells, about 4.3 CGAC/10,000 cells, about 4.4 CGAC/10,000 cells, about 4.5 CGAC/10,000 cells, about 4.6 CGAC/10,000 cells, about 4.7 CGAC/10,000 cells, about 4.8 CGAC/10,000 cells, about 4.9 CGAC/10,000 cells, about 5.0 CGAC/10,000 cells, about 5.5 CGAC/10,000 cells, about 6.0 CGAC/10,000 cells, about 6.5 CGAC/10,000 cells, about 7.0 CGAC/10,000 cells, about 7.5 CGAC/10,000 cells, about 8.0 CGAC/10,000 cells, about 8.5 CGAC/10,000 cells, about 9.0 CGAC/10,000 cells, about 9.5 CGAC/10,000 cells, about 10.0 CGAC/10,000 cells, about 15.0 CGAC/10,000 cells, about 20.0 CGAC/10,000 cells, about 30.0 CGAC/10,000 cells, about 40.0 CGAC/10,000 cells, about 50.0 CGAC/10,000 cells, about 60.0 CGAC/10,000 cells, about 70.0 CGAC/10,000 cells, about 80.0 CGAC/10,000 cells, about 90.0 CGAC/10,000 cells, about 100 CGAC/10,000 cells, about 150 CGAC/10,000 cells, about 200 CGAC/10,000 cells, about 300 CGAC/10,000 cells, about 400 CGAC/10,000 cells, about 500 CGAC/10,000 cells, about 600 CGAC/10,000 cells, about 700 CGAC/10,000 cells, about 800 CGAC/10,000 cells, about 900 CGAC/10,000 cells, about 1,000 CGAC/10,000 cells, about 2,000 CGAC/10,000 cells, or about 3,000 CGAC/10,000 cells, (or any number in between).

In some aspects, a sample score greater than 0.5 CGAC/10,000 cells represents a risk of lung cancer. In some aspects, a sample score greater than 1 CGAC/10,000 cells represents a risk of lung cancer. In some aspects, a sample score greater than 2 CGAC/10,000 cells represents a risk of lung cancer. In some aspects, a sample score greater that 2.2 CGAC/10,000 cells represents a risk of lung cancer. In some aspects, a sample score greater that 2.4 CGAC/10,000 cells represents a risk of lung cancer. In some aspects, a sample score greater that 2.5 CGAC/10,000 cells represents a risk of lung cancer. In some aspects, a sample score greater that 2.6 CGAC/10,000 cells represents a risk of lung cancer. In some aspects, a sample score greater that 2.8 CGAC/10,000 cells represents a risk of lung cancer. In some aspects, a sample score greater than 3 CGAC/10,000 cells represents a risk of lung cancer. In some aspects, a sample score greater than 4 CGAC/10,000 cells represents a risk of lung cancer. In some aspects, a sample score greater than 5 CGAC/10,000 cells represents a risk of lung cancer. In some aspects, a sample score greater than 10 CGAC/10,000 cells represents a risk of lung cancer. In some aspects, a sample score greater than 20 CGAC/10,000 cells represents a risk of lung cancer.

In some aspects, a sample score greater than 0.5 CGAC/10,000 cells is indicative of a subject having lung cancer. In some aspects, a sample score greater than 1 CGAC/10,000 cells is indicative of a subject having lung cancer. In some aspects, a sample score greater than 2 CGAC/10,000 cells is indicative of a subject having lung cancer. In some aspects, a sample score greater that 2.2 CGAC/10,000 cells is indicative of a subject having lung cancer. In some aspects, a sample score greater that 2.4 CGAC/10,000 cells is indicative of a subject having lung cancer. In some aspects, a sample score greater that 2.5 CGAC/10,000 cells is indicative of a subject having lung cancer. In some aspects, a sample score greater that 2.6 CGAC/10,000 cells is indicative of a subject having lung cancer. In some aspects, a sample score greater that 2.8 CGAC/10,000 cells is indicative of a subject having lung cancer. In some aspects, a sample score greater than 3 CGAC/10,000 cells is indicative of a subject having lung cancer. In some aspects, a sample score greater than 4 CGAC/10,000 cells is indicative of a subject having lung cancer. In some aspects, a sample score greater than 5 CGAC/10,000 cells is indicative of a subject having lung cancer. In some aspects, a sample score greater than 10 CGAC/10,000 cells is indicative of a subject having lung cancer. In some aspects, a sample score greater than 20 CGAC/10,000 cells is indicative of a subject having lung cancer.

In some aspects, if 1 advanced CGAC is identified per 10,000 cells, the subject is identified as having lung cancer.

In some aspects, a subject with a sample score greater than the predetermined cutoff value is referred for surgical resection of a pulmonary nodule or tumor. In some aspects, the subject with a sample score greater than 5 CGAC/10,000 cells is referred for surgical resection of a pulmonary nodule or tumor.

Methods of the disclosure further comprise administering one or more therapeutic agents suitable for the treatment of lung cancer to a subject who has been diagnosed with lung cancer according to methods disclosed herein.

The disclosure provides methods of evaluating cancer in a subject comprising determining the level of CGACs, or the sample score, in a sample containing blood cells from the patient by methods of the disclosure, wherein a higher level of CGACs, or higher sample score, in the sample, as compared to a control or predetermined number of CGACs from a non-aggressive form of cancer, is indicative of an aggressive form of cancer and/or a poor cancer prognosis.

The disclosure provides methods of staging cancer in a subject comprising determining CGACs in a sample containing blood cells from the subject by methods of the disclosure, wherein a higher level of CGACs, or higher sample score, in the sample as compared to a predetermined control for a given stage is indicative of a more advanced stage of cancer, and a lower level of CGACs in the sample as compared to a control for a given stage is indicative of a less advanced stage of cancer.

### Cancer

The present disclosure envisions the use of assays to detect cancer and predict its progression in conjunction with cancer therapies. In some cases, where patients are suspected to be at risk of cancer, prophylactic treatments may be employed. In other cancer subjects, diagnosis may permit early therapeutic intervention. In yet other situations, the result of the assays described herein may provide useful information regarding the need for repeated treatments, for example, where there is a likelihood of metastatic, recurrent, or residual disease. Finally, the present disclosure may prove useful in demonstrating which therapies do and do not provide benefit to a particular patient.

Furthermore, the methods described in this application are able to be translated into a method for isolating CGAC and circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc.

The current invention is useful for the prognosis and diagnosis of lung cancers, which can be defined by a number of histologic classifications including: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma. Diagnosis and prognosis of other smoking related cancers is possible with these probes. Squamous cell carcinoma of the head and neck has the same risk factors as lung cancer and is hypothesized to have similar etiology (Shriver, 1998). Similarly, smoking is an etiological factor for cancer of the bladder, head, neck, kidneys, pancreas, and cancer of the upper airways including cancer of the mouth, throat, pharynx, larynx, or esophagus.

### A. Tumorigenesis

The deletion of various genes in tumor tissue has been well studied in the art. However, there remains a need for probes that are significant for detecting early molecular events in the development of cancers, as well as molecular events that make patients susceptible to the development of cancer. Probes used for the staging of cancer are also of interest. The proposed sequence leading to tumorigenesis includes genetic instability at the cellular or submicroscopic level as demonstrated by loss or gain of chromosomes, leading to a hyperproliferative state due to theoretical acquisition of factors that confer a selective proliferative advantage. Further, at the genetic level, loss of function of cell cycle inhibitors and tumor suppressor genes (TSG), or amplification of oncogenes that drive cell proliferation, are implicated.

Following hyperplasia, a sequence of progressive degrees of dysplasia, carcinoma-in-situ and ultimately tumor invasion is recognized on histology. These histologic changes are both preceded and paralleled by a progressive accumulation of genetic damage. At the chromosomal level genetic instability is manifested by a loss or gain of chromosomes, as well as structural chromosomal changes such as translocation and inversions of chromosomes with evolution of marker chromosomes. In addition, cells may undergo polyploidization. Single or multiple clones of neoplastic cells may evolve characterized in many cases by aneuploid cell populations. These can be quantitated by measuring the DNA content or ploidy relative to normal cells of the patient by techniques such as flow cytometry or image analysis.

### B. Prognostic Factors and Staging

The stage of a cancer at diagnosis is an indication of how much the cancer is spread and can be one of the most important prognostic factors regarding patient survival. Staging systems are specific for each type of cancer. For example, at present the most important prognostic factor regarding the survival of patients with lung cancer of non-small cell type is the stage of disease at diagnosis. For example, the most important prognostic factor regarding the survival of patients with lung cancer of non-small cell type is the stage of disease at diagnosis. Conversely, small cell cancer usually presents with widespread dissemination hence the staging system is less applicable. The staging system was devised based on the anatomic extent of cancer and is now known as the TNM (Tumor, Node, Metastasis) system based on anatomical size and spread within the lung and adjacent structures, regional lymph nodes and distant metastases. The only hope presently for a curative procedure lies in the operability of the tumor which can only be resected when the disease is at a low stage when confined to the organ of origination.

### C. Grading of Tumors

The histological type and grade of lung cancers do have some prognostic impact within the stage of disease with the best prognosis being reported for stage I adenocarcinoma, with 5 year survival at 50% and 1-year survival at 65% and 59% for the bronchiolar-alveolar and papillary subtypes (Naruke et al., 1988; Travis et al., 1995; Carriaga et al., 1995). For squamous cell carcinoma and large cell carcinoma the 5 year survival is around 35%. Small cell cancer has the worst prognosis with a 5 year survival rate of only 12% for patients with localized disease (Carcy et al., 1980; Hirsh, 1983; Vallmer et al., 1985). For patients with distant metastases survival at 5 years is only 1-2% regardless of histological subtype (Naruke et al., 1988). In addition to histological subtype, it has been shown that histological grading of carcinomas within subtype is of prognostic value with well differentiated tumors having a longer overall survival than poorly differentiated neoplasms. Well differentiated localized adenocarcinoma has a 69% overall survival compared to a survival rate of only 34% of patients with poorly differentiated adenocarcinoma (Hirsh, 1983). The 5 year survival rates of patients with localized squamous carcinoma have varied from 37% for well differentiated neoplasms to 25% for poorly differentiated squamous carcinomas (Ihde, 1991).

The histologic criteria for subtyping lung tumors are as follows: squamous cell carcinoma consists of a tumor with keratin formation, keratin pearl formation, and/or intercellular bridges. Adenocarcinomas consist of a tumor with definitive gland formation or mucin production in a solid tumor. Small cell carcinoma consists of a tumor composed of small cells with oval or fusiform nuclei, stippled chromatin, and indistinct nuclei. Large cell undifferentiated carcinoma consists of a tumor composed of large cells with vesicular nuclei and prominent nucleoli with no evidence of squamous or glandular differentiation. Poorly differentiated carcinoma includes tumors containing areas of both squamous and glandular differentiation.

### D. Development of Carcinomas

The evolution of carcinoma of the lung is most likely representative of a field cancerization effect as a result of the entire aero-digestive system being subjected to a prolonged period of carcinogenic insults such as benzylpyrenes, asbestosis, air pollution and chemicals other carcinogenic substances in cigarette smoke or other environmental carcinogens. This concept was first proposed by Slaughter et al. (1953). Evidence for existence of a field effect is the common occurrence of multiple synchronous for metachronous second primary tumors (SPTs) that may develop throughout the aero-digestive tract in the oropharynx, upper esophagus or ipsilateral or contralateral lung.

Accompanying these molecular defects is the frequent manifestation of histologically abnormal epithelial changes including hyperplasia, metaplasia, dysplasia, and carcinoma-in-situ. It has been demonstrated in smokers that both the adjacent normal bronchial epithelium as well as the preneoplastic histological lesions may contain clones of genetically altered cells (Wistuba et al., 2000).

Licciardello et al. (1989) found a 10-40% incidence of metachronous tumors and a 9-14% incidence of synchronous SPTs in the upper and lower aero-digestive tract, mostly in patients with the earliest primary tumors SPTs may impose a higher risk than relapse from the original primary tumor and may prove to be the major threat to long term survival following successful therapy for early stage primary head, neck, or lung tumors. Hence it is vitally important to follow these patients carefully for evidence of new SPTs in at risk sites for new malignancies specifically in the aero-digestive system.

In addition to chromosomal changes at the microscopic level, multiple blind bronchial biopsies may demonstrate various degrees of intraepithelial neoplasia at loci adjacent to the areas of lung cancer. Other investigators have shown that there are epithelial changes ranging from loss of cilia and basal cell hyperplasia to CIS in most light and heavy smokers and all lungs that have been surgically resected for cancer (Auerbach et al., 1961). Voravud et al. (1993) demonstrated by in-situ hybridization (ISH) studies using chromosome-specific probes for chromosomes 7 and 17 that 30-40% of histologically normal epithelium adjacent to tumor showed polysomies for these chromosomes. In addition, there was a progressive increase in frequency of polysomies in the tissue closest to the carcinoma as compared to normal control oral epithelium from patients without evidence of carcinoma. The findings of genotypic abnormalities that increased closer to the area of the tumor support the concept of field cancerization. Interestingly, there was no increase in DNA content as measured in the normal appearing mucosa in a Feulgen stained section adjacent to the one where the chromosomes were measured, reflecting perhaps that insufficient DNA had been gained in order to alter the DNA index. Interestingly, a very similar increase in DNA content was noted both in dysplastic areas close to the cancer and in the cancerous areas suggesting that complex karyotypic abnormalities that are clonal have already been established in dysplastic epithelium adjacent to lung cancer. Others have also shown an increase in the number of cells showing p53 mutations in dysplastic lesions closest to areas of cancer, which are invariably also p53 mutated. Other chromosomal abnormalities that have recently been demonstrated in tumors and dysplastic epithelium of smokers includes deletions of 3p, 17p, 9 p and 5q (Feder et al., 1998; Yanagisawa et al., 1996; Thiberville et al., 1995).

### E. Chromosome Deletions in Lung Cancer

Small cell lung cancer (SCLC) and non-small cell lung cancer commonly display cytogenetically visible deletions on the short arm of chromosome 3 (Hirano et al., 1994; Valdivieso et al., 1994; Cheon et 41993; Pence et al., 1993). This 3p deletion occurs more frequently in the lung tumor tissues of patients who smoke than it does in those of nonsmoking patients. (Rice et al., 1993) Since approximately 85% lung cancer patients were heavy cigarette smokers (Mrkve et al., 1993), 3p might contain specific DNA loci related to the exposure of tobacco carcinogens. It also has been reported that 3p deletion occurs in the early stages of lung carcinogenesis, such as bronchial dysplasia (Pantel et al., 1993). In addition to cytogenetic visible deletions, loss of heterozygosity (LOH) studies have defined 3-21.3 as one of the distinct regions that undergo loss either singly or in combination (Fontanini et al., 1992; Liewald et al., 1992). Several other groups have found large homozygous deletions at 3p21.3 in lung cancer (Macchiarini et al., 1992; Miyamoto et al., 1991; Ichinose et al., 1991; Yamaoka et al., 1990). Transfer of DNA fragments from 3-21.3-3p21.2 into lung tumor cell lines could suppress the tumorigenesis (Sahin et al., 1990; Volm et al., 1989). These findings strongly suggest the presence of at least one tumor suppressor gene in this specific chromosome region whose loss will initiate lung carcinogenesis.

Cytogenetic observation of lung cancer has shown an unusual consistency in the deletion rate of chromosome 3p. In fact, small cell lung cancer (SCLC) demonstrates a 100% deletion rate within certain regions of chromosome 3p. non-small cell lung cancer (NSCLC) demonstrates a 70% deletion rate (Mitsudomi et al., 1996; Shiseki et al., 1996). Loss of heterozygosity and comparative genomic hybridization analysis have shown deletions between 3p14.2 and 3p21.3 to be the most common finding for lung carcinoma and is postulated to be the most crucial change in lung tumorigenesis (Wu et al., 1998). It has been hypothesized that band 3p21.3 is the location for lung cancer tumor suppressor genes. The hypothesis is supported by chromosome 3 transfer studies, which reduced tumorigenicity in lung adenocarcinoma.

Allelotype studies on non-small cell lung carcinoma indicated loss of genetic material on chromosome 10q in 27% of cases. Studies of chromosome 10 allelic loss have shown that there is a very high incidence of LOH in small cell lung cancer, up to 91% (Alberola et al., 1995; Ayabe et al., 1994). A statistically significant LOH of alleles on 10q was noted in metastatic squamous cell carcinoma (SCC) in 56% of cases compared to non-metastatic SCC with LOH seen in only 14% of cases (Ayabe et al., 1994). No LOH was seen in other subtypes on NSCLC. Additionally, using microsatellite polymorphism analysis, it was shown that a high incidence of loss exists between D10s677 and D1051223. This region spans the long arm of chromosome 10 at bands q21-q24 and overlaps the region deleted in the study of advanced stage high grade bladder cancers which demonstrated a high frequency of allele loss within a 2.5 cM region at 10q22.3-10q23.1 (Kim et al., 1996).

### Gene Probes

The present disclosure comprises contacting the selected cells with a labeled nucleic acid probe, and detecting hybridized cells by fluorescence in situ hybridization. These probes may be specific for any genetic marker that is most frequently amplified or deleted in CGACs. In particular, the probes may be a 3p22.1 probe, which is a nucleic acid probe targeting RPL14, CD39L3, PMGM, or GC20, combined with centromeric 3; a 10q22-23 probe (encompassing surfactant protein A1 and A2) combined with centromeric 10; or a PI3 kinase probe. Other genetic markers may include, but are not limited to, centromeric 3, 7, 17, 9p21, 5p15.2, EGFR, C-myc8q22, and 6p22-22. For a further discussion of gene probes see U.S. Publication No. 2007/0218480, herein incorporated by reference in its entirety.

### 3p22.1 Gene Probes

A 3p22.1 probe is a nucleic acid probe targeting RPL14, CD39L3, PMGM, or GC20, combined with centromeric 3. The human ribosomal L14 (RPL14) gene (GenBank Accession NM_003973), and the genes CD39L3 (GenBank Accession AAC39884 and AF039917), PMGM (GenBank Accession P15259 and J05073), and GC20 (GenBank Accession NM_005875) were isolated from a BAC (GenBank Accession AC104186, herein incorporated by reference) and located in the 3p22.1 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. It is understood that the polyalanine tract plays a key role in the nonsense-mediated mRNA decay pathway that rids cells aberrant proteins and transcripts. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. See also RP11-391M1/AC104186.

Genes with a regulatory function such as the RPL14 gene, along with the genes CD39L3, PMGM, and GC20 and analogs thereof, are good candidates for diagnosis of tumorigenic events. It has been postulated that functional changes of the RPL14 protein can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

### 3p21.3 Gene Probes

### Structural Features

Recently, the human ribosomal L14 (RPL14) gene (GenBank Accession NM_003973), and the genes CD39L3 (GenBank Accession AAC39884 and AF039917), PMGM (GenBank Accession P15259 and J05073), and GC20 (GenBank Accession NM 005875) were isolated from a BAC (GenBank Accession AC019204, herein incorporated by reference) and located in the 3p21.3 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population.

### Functional Aspects

Genes with a regulatory function such as the RPL14 gene, along with the genes CD39L3, PMGM, and GC20 and analogs thereof, are good candidates for diagnosis of tumorigenic events. It has been postulated that functional changes of the RPL14 protein can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

### 10q22 Gene Probes

### Structural Features

In other embodiments, the probe may be a 10q22-23 probe, which encompasses surfactant protein A1 and A2, combined with centromeric 10. The 10q22 BAC (46b12) is 200 Kb and is adjacent and centromeric to PTEN/MMAC1 (GenBank Accession AF067844), which is at 10q22-23 and can be purchased through Research Genetics (Huntsville, Ala.). Alterations to 10q22-25 has been associated with multiple tumors, including lung, prostate, renal, and endometrial carcinomas, melanoma, and meningiomas, suggesting the possible suppressive locus affecting several cancers in this region. The PTEN/MMAC1 gene, encoding a dual-specificity phosphatase, is located in this region, and has been isolated as a tumor suppressor gene that is altered in several types of human tumors including brain, bladder, breast, and prostate cancers. PTEN/MMAC1 mutations have been found in some cancer cell lines, xenografts, and hormone refractory cancer tissue specimens. Because the inventor's 10q22 BAC DNA sequence is adjacent to this region, the DNA sequences in the BAC 10q22 may be involved in the genesis and/or progression of human lung cancer. See also RP11-506M13/AC068139.6

Pulmonary-associated surfactant protein A1 (SP-A) is located at 10q22.3. Surfactant protein-A-phospholipid-protein complex lowers the surface tension in the alveoli of the lung and plays a major role in host defense in the lung. Surfactant protein-A1 is also present in alveolar type-2 cells, which are believed to be putative stem cells of the lung. It is known that type-2 cells participate in repair and regeneration after alveolar damage. Thus, it is possible that the type-2 cells express telomerase and C-MYC, which leads to the loss of the surfactant protein and the development of non-small cell lung cancer. The 10q22 probe is useful in the further development of clinical biomarkers for the early detection of neoplastic events, for risk assessment and monitoring the efficacy of chemoprevention therapy.

### Functional Aspects

Functional evidence for the presence of tumor suppressor genes on 10q has been provided by microcell-mediated chromosomal transfer. The resulting hybrid clones displayed a suppressed tumorigenic phenotype with the inability to proliferate in nude mice and soft agarose. Sequence analysis of the PTEN/MMAC1 gene in lung cancer revealed a G to C substitution located 8 bp upstream of the coding region of exon1 and which seems to be a polymorphism, in 4 of the 30 cases of lung cancer tested. Somatic mutations of the TPEN/MMAC1 gene were not identified in any of the tumors at the primary and metastatic sites of lung cancer, indicating that point mutations in the PTEN/MMAC1 gene are probably not an important factor in tumorigenesis and the progression of a major subset of lung cancers. Other more important tumor suppressor genes must lie close to the PTEN/MMAC1 gene, in the vicinity of the inventors' 10q22 BAC locus. Therefore, the 10q22 probe is useful in the further development of clinical biomarkers for the early detection of neoplastic events, for risk assessment and monitoring the efficacy of chemoprevention therapy in high risk former or current smokers.

### C. Commercial Probe Sets

Any commercial probes or probe sets may also be used with the present disclosure. For example, the UroVysion DNA probe set (Vysis/Abbott Molecular, Des Plaines, Ill.) may be used, which includes probes directed to centromeric 3, centromeric 7, centromeric 17, 9p21.3. It has been established that UroVysion probes detect early changes of lung cancer. In other embodiments, the LaVysion DNA probe set (Vysis/Abbott Molecular, Des Plaines, Ill.), which includes probes to 7p12 (epidermal growth factor receptor); 8q24.12-q24.13 (MYC); 6p11.1-q11 (chromosome enumeration (Probe CEP 6); and 5p15.2 (encompassing the SEMA5A gene), may be used. It has been noted that the LaVysion probe set detects higher stages or more advanced stages of lung cancer. Furthermore, a single probe set directed to centromeric 7/7p12 (epidermal growth factor receptor) may also be used with the present disclosure.

### Methods for Assessing Gene Structure

In accordance with the present disclosure, one will utilize various probes to examine the structure of genomic DNA from patient samples. A wide variety of methods may be employed to detect changes in the structure of various chromosomal regions. The following is a non-limiting discussion of such methods.

### A. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non-fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout its length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.). Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for loss of allelic heterozygosity is that the:
(a) FISH is easily and rapidly performed on cells of interest and can be used on paraffin-embedded, or fresh or frozen tissue allowing the use of micro-dissection;
(b) specific gene changes can be analyzed on a cell by cell basis in relationship to centromeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted);
(c) PCR cannot identify amplification of genes; and
(d) FISH using bacterial artificial chromosomes (BACs) permits easy detection and localization on specific chromosomes of genes of interest which have been isolated using specific primer pairs.

Chromogenic in situ hybridization (CISH) enables the gain of genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference. The scanning may be performed, for example, on an automated scanner with Fluorescence capabilities (Bioview System, Rehovot, Israel).

### B. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure (Wu et al., 1989, incorporated herein by reference in its entirety).

### C. Southern Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

### D. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present disclosure: adsorption, partition, ion-exchange and molecular sieve, and many specialized techniques for using them including column, paper, thin-layer and gas chromatography (Freifelder, 1982).

### E. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present disclosure provides methods by which any or all of these types of analyses may be used.

### F. Kit Components

All the essential materials and reagents required for detecting changes in the chromosomal regions discussed above may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification, and optionally labeling agents such as those used in FISH. Such kits also generally will comprise, in suitable means, distinct containers for each individual reagent and enzyme as well as for each primer or probe.

### G. Chip Technologies

Specifically contemplated by the present inventors are chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules using methods such as fluorescence, conductance, mass spectrometry, radiolabeling, optical scanning, or electrophoresis. See also Pease et al. (1994); Fodor et al. (1991).

Biologically active DNA probes may be directly or indirectly immobilized onto a surface to ensure optimal contact and maximum detection. When immobilized onto a substrate, the gene probes are stabilized and therefore may be used repetitively. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Immobilization of the gene probes may be achieved by a variety of methods involving either non-covalent or covalent interactions between the immobilized DNA comprising an anchorable moiety and an anchor. DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. Gene probe may be bound directly to membranes using ultraviolet radiation. With nitrocellous membranes, the probes are spotted onto the membranes. A UV light source is used to irradiate the spots and induce cross-linking. An alternative method for cross-linking involves baking the spotted membranes at 80° C. for two hours in vacuum.

Immobilization can consist of the non-covalent coating of a solid phase with streptavidin or avidin and the subsequent immobilization of a biotinylated polynucleotide (Holmstrom, 1993). Precoating a polystyrene or glass solid phase with poly-L-Lys or poly L-Lys, Phe, followed by the covalent attachment of either amino- or sulfhydryl-modified polynucleotides using bifunctional crosslinking reagents (Running, 1990; Newton, 1993) can also be used to immobilize the probe onto a surface.

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287) describes a method of non-covalently immobilizing nucleic acid molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the synthetic nucleic acid and the cationic detergent or salt. The support containing the immobilized nucleic acid may be washed with an aqueous solution containing a non-ionic detergent without removing the attached molecules.

There are two common variants of chip-based DNA technologies involving DNA microarrays with known sequence identity. For one, a probe cDNA (500˜5,000 bases long) is immobilized to a solid surface such as glass using robot spotting and exposed to a set of targets either separately or in a mixture. This method, “traditionally” called DNA microarray, is widely considered as developed at Stanford University. A recent article by Ekins and Chu (1999) provides some relevant details. The other variant includes an array of oligonucleotide (20˜25-mer oligos) or peptide nucleic acid (PNA) probes synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its products under the GeneChip® trademark.

### Nucleic Acids

The inventors provide a method comprising a step of contacting the selected cells with a labeled nucleic acid probe forming hybridized cells, wherein hybridization of the labeled nucleic acid is indicative of a CGAC. However, the present disclosure is not limited to the use of the specific nucleic acid segments disclosed herein. Rather, a variety of alternative probes that target the same regions/polymorphisms may be employed.

### Probes and Primers

Naturally, the present disclosure encompasses DNA segments that are complementary, or essentially complementary, to target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a target nucleic acid segment under relatively stringent conditions such as those described herein. These probes may span hundreds or thousands of base pairs.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of about 8, 9, 10, 11, 12, 13, 14, 15, 16, 17, 18, 19, 20, 25, 30, 35, 40, 45, 50, 55, 60, 65, 70, 75, 80, 85, 90, 95, 100, 250, 500, 700, 722, 900, 992, 1000, 1500, 2000, 2500, 2800, 3000, 3500, 3800, 4000, 5000 or more base pairs will be used, although others are contemplated. As mentioned above, longer polynucleotides encoding 10,000, 50,000, 100,000, 150,000, 200,000, 250,000, 300,000 and 500,000 bases are contemplated. Such oligonucleotides and polynucleotides will find use, for example, as probes in FISH, Southern and Northern blots and as primers in amplification reactions.

It will be understood that this disclosure is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present disclosure are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

### Labeling of Probes

In certain embodiments, it will be advantageous to employ nucleic acid sequences of the present disclosure in combination with an appropriate means, such as a label, for determining hybridization. A wide variety of appropriate indicator means are known in the art, including fluorescent, radioactive, chemiluminescent, electroluminescent, enzymatic tag or other ligands, such as avidin/biotin, antibodies, affinity labels, etc., which are capable of being detected. In preferred embodiments, one may desire to employ a fluorescent label such as digoxigenin, spectrum orange, fluorescein, eosin, an acridine dye, a rhodamine, Alexa 350, Alexa 430, AMCA, BODIPY 630/650, BODIPY 650/665, BODIPY-FL, BODIPY-R6G, BODIPY-TMR, BODIPY-TRX, cascade blue, Cy2, Cy3, Cy5,6-FAM, HEX, 6-JOE, Oregon green 488, Oregon green 500, Oregon green 514, pacific blue, REG, ROX, TAMRA, TET, or Texas red.

In the case of enzyme tags such as urease alkaline phosphatase or peroxidase, colorimetric indicator substrates are known which can be employed to provide a detection means visible to the human eye or spectrophotometrically, to identify specific hybridization with complementary nucleic acid-containing samples. Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

The indicator means may be attached directly to the probe, or it may be attached through antigen bonding. In preferred embodiments, digoxigenin is attached to the probe before denaturation and a fluorophore labeled anti-digoxigenin FAB fragment is added after hybridization.

### Hybridization Conditions

Suitable hybridization conditions will be well known to those of skill in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl2, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl2, at temperatures ranging from approximately 40° C. to about 72° C. Formamide and SDS also may be used to alter the hybridization conditions.

### Biomarkers and Other Risk Factors

Various biomarkers of prognostic significance can be used in conjunction with the specific nucleic acid probes discussed above. These biomarkers could aid in predicting the survival in low stage cancers and the progression from preneoplastic lesions to invasive lung cancer. These markers can include proliferation activity as measured by Ki-67 (MIB1), angiogenesis as quantitated by expression of VEGF and microvessels using CD34, oncogene expression as measured by erb B2, and loss of tumor suppresser genes as measured by p53 expression.

Multiple biomarker candidates have been implicated in the evolution of neoplastic lung lesions. Bio-markers that have been studies include general genomic markers including chromosomal alterations, specific genomic markers such as alterations in proto-oncogenes such as K-Ras, Erbβ1/EGFR, Cyclin D; proliferation markers such as Ki67 or PCNA, squamous differentiation markers, and nuclear retinoid receptors (Papadimitrakopoulou et al., 1996) The latter are particularly interesting as they may be modulated by specific chemopreventive drugs such as 13-cis-retinoic acid or 4HTPR and culminate in apoptosis of the defective cells with restoration of a normally differentiated mucosa (Zou et al., 1998).

### Tumor Angiogenesis by Microvessel Counts

Tumor angiogenesis can be quantitated by microvessel density and is a viable prognostic factor in stage 1 NSCLC. Tumor microvessel density appears to be a good predictor of survival in stage 1 NSCLC.

### Vascular Endothelial Growth Factor (VEGF)

VEGF (3,6-8 ch 4) an endothelial cell specific mitogen is an important regulator of tumor angiogenesis who's expression correlates well with lymph node metastases and is a good indirect indicator of tumor angiogenesis. VEGF in turn is upregulated by P53 protein accumulation in NSCLC.

p53

The role of p53 mutations in predicting progression and survival of patients with NSCLC is widely debated. Although few studies imply a negligible role, the majority of the studies provide compelling evidence regarding the role of p53 as one of the prognostic factors in NSCLC. The important role of p53 in the biology of NSCLC has been the basis for adenovirus mediated p53 gene transfer in patients with advanced NSCLC (Carcy et al., 1980). In addition p53 has also been shown to be an independent predictor of chemotherapy response in NSCLC. In a recent study (Vallmer et al., 1985), the importance of p53 accumulation in preinvasive bronchial lesions from patients with lung cancer and those who did not progress to cancer were studied. It was demonstrated that p53 accumulation in preneoplastic lesions had a higher rate of progression to invasion than did p53 negative lesions.

c-erb-B2

Similar to p53, c-erg-B2 (Her2/neu) expression has also been shown to be a good marker of metastatic propensity and an indicator of survival in these tumors.

### Ki-67 Proliferation Marker

In addition to the above markers, tumor proliferation index as measured by the extent of labeling of tumor cells for Ki-67, a nuclear antigen expressed throughout cell cycle correlates significantly with clinical outcome in Stage 1 NSCLC (Feinstein et al., 1970). The higher the tumor proliferation index the poorer is the disease free survival labeling indices provide significant complementary, if not independent prognostic information in Stage 1 NSCLC, and helps in the identification of a subset of patients with Stage 1 NSCLC who may need more aggressive therapy.

Alterations in the 3p21.3 and 10q22 loci are known to be associated with a number of cancers. More specifically, point mutations, deletions, insertions or regulatory perturbations relating to the 3p21.3 and 10q22 loci may cause cancer or promote cancer development, cause or promoter tumor progression at a primary site, and/or cause or promote metastasis. Other phenomena at the 3p21.3 and 10q22 loci include angiogenesis and tissue invasion. Thus, the present inventors have demonstrated that deletions at 3p21.3 and 10q22 can be used not only as a diagnostic or prognostic indicator of cancer, but to predict specific events in cancer development, progression and therapy.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited.

### Surfactant Protein A and B

There are four main surfactant proteins: SP-A, B, C, and D. SP-A and D are hydrophilic, while SP-B and C are hydrophobic. The proteins are very sensitive to experimental conditions (temperature, pH, concentration, substances such as calcium, and so on). Moreover, their effects tend to overlap and thus it is difficult to pinpoint the specific role of each protein.

### SP-A

SP-A was the first surfactant protein to be identified, and is also the most abundant (Ingenito et al., 1999). Its molecular mass varies from 26-38 kDa (Pérez-Gil et al., 1998). The protein has a “bouquet” structure of six trimers (Haagsman and Diemel, 2001), and can be found in an open or closed form depending on the other substances present in the system. Calcium ions produce the closed-bouquet form (Palaniyar et al., 1998).

SP-A plays a role in immune defense. It is also involved in surfactant transport/adsorption (with other proteins). SP-A is necessary for the production of tubular myelin, a lipid transport structure unique to the lungs. Tubular myelin consists of square tubes of lipid lined with protein (Palaniyar et al., 2001). Mice genetically engineered to lack SP-A have normal lung structure and surfactant function, and it is possible that SP-A's beneficial surfactant properties are only evident under situations of stress (Korfhagen et al., 1996).

### SP-B

Papillary thyroid carcinoma (PTC) is clinically heterogeneous. Apart from an association with ionizing radiation, the etiology and molecular biology of PTC is poorly understood. Using oligo-based DNA arrays to study expression profiles of eight matched pairs of normal thyroid and PTC tissues, Immunohistochemical analysis detected SFTPB in 39/52 PTCs, but not in follicular thyroid carcinoma and normal thyroid tissue. Huang et al. (2001.

### Patient Interview and Other Risk Factors

In addition to analyzing the presence or absence of polymorphisms, as discussed above, it may be desirable to evaluate additional factors in a patient. For example, a patient interview, which would include a smoking history (years smoking, pack/day, etc.) is highly relevant to the diagnosis/prognosis. Also, the presence or absence of morphologic changes in sputum cells (squamous metaplasia, dysplasia, etc.) and a genetic instability score (genetic instability=composing the sum of abnormalities from various combinations in epithelial and neutrophils in sputum and/or peripheral blood cells or bone marrow cells or stem cells isolated from blood or bone marrow) may be used.

### Obtaining and Purifying Samples

In accordance with the present disclosure, one will obtain a biological sample that contains blood cells. In some embodiments, the entity evaluating the sample for CGAC levels did not directly obtain the sample from the patient. Therefore, methods of the disclosure involve obtaining the sample indirectly or directly from the patient. To achieve these methods, a doctor, medical practitioner, or their staff may obtain a biological sample for evaluation. The sample may be analyzed by the practitioner or their staff, or it may be sent to an outside or independent laboratory. The medical practitioner may be cognizant of whether the test is providing information regarding a quantitative level of CGACs.

In any of these circumstances, the medical practitioner may know the relevant information that will allow him or her to determine whether the patient can be diagnosed as having an aggressive form of cancer and/or a poor cancer prognosis based on the level of CGACs. It is contemplated that, for example, a laboratory conducts the test to determine the level of CGACs. Laboratory personnel may report back to the practitioner with the specific result of the test performed.

Typically, the sample is isolated from a biological sample taken from the individual, such as a blood sample or tissue sample using standard techniques such as disclosed in Jones (1963) which is hereby incorporated by reference. Collection of the samples may be by any suitable method, although in some aspects collection is by needle, catheter, syringe, scrapings, and so forth.

The sample may be prepared in any manner known to those of skill in the art. For example, the circulating epithelial cells from peripheral blood may be isolated from the buffy layer following Ficoll-Hypaque gradient separation, allowing for enrichment of mononuclear cells (lymphocytes and epithelial cells). Other methods known to those of skill in the art may also be used to prepare the sample.

Nucleic acids may be isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. Depending on the format, the specific nucleic acid of interest is identified in the sample directly using amplification or with a second, known nucleic acid following amplification.

Following detection, one may compare the results seen in a given sample with a statistically significant reference group of samples from normal patients and patients that have or lack alterations in the various chromosome loci and control regions. In this way, one then correlates the amount or kind of alterations detected with various clinical states and treatment options.

### Cancer Treatments

In some embodiments, the disclosure provides compositions and methods for the diagnosis and treatment of lung cancer. In one embodiment, the disclosure provides a method of determining the treatment of cancer based on whether the level of CGACs is high in comparison to a control. The treatment may be a conventional cancer treatment. One of skill in the art will be aware of many treatments that may be combined with the methods of the present disclosure, some but not all of which are described below.

### A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the disclosure, methods include identifying a patient in need of treatment. A patient may be identified, for example, based on taking a patient history or based on findings on clinical examination.

### B. Treatments

In some embodiments, the method further comprises treating a patient with lung cancer with a conventional cancer treatment. One goal of current cancer research is to find ways to improve the efficacy of chemo- and radiotherapy, such as by combining traditional therapies with other anti-cancer treatments. In the context of the present disclosure, it is contemplated that this treatment could be, but is not limited to, chemotherapeutic, radiation, a polypeptide inducer of apoptosis, a novel targeted therapy such as a tyrosine kinase inhibitor, or an anti-VEGF antibody, or other therapeutic intervention. It also is conceivable that more than one administration of the treatment will be desired.

### 1. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gene silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance antitumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and re-administered (Rosenberg et al., 1988; 1989).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Gene Therapy

In yet another embodiment, the secondary treatment is a gene therapy in which a therapeutic polynucleotide is administered before, after, or at the same time as a H2A.Z targeting agent is administered. Delivery of a H2A.Z targeting agent in conjunction with a vector encoding one of the following gene products may have a combined anti-hyperproliferative effect on target tissues. A variety of proteins are encompassed within the disclosure, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present disclosure, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present disclosure include Rb, APC, DCC, NF-1, NF-2, WT-1, MEN-I, MEN-II, zac1, p73, VHL, MMAC1/H2A.Z, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and the ICE-like proteases have both been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BlcW, BlcS, Mcl-1, A1, Bfl-1) or counteract Bcl-2 function and promote cell death (e.g., Bax, Bak, Bik, Bim, Bid, Bad, Harakiri).

d. RNA Interference (RNAi)

In certain embodiments, the H2A.Z inhibitor is a double-stranded RNA (dsRNA) directed to an mRNA for H2A.Z.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

e. siRNA

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

f. Production of Inhibitory Nucleic Acids

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedure may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25-mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ^{˜}21 nt siRNA-like molecules (Brummelkamp et al., 2002). The siRNA-like molecules can, in turn, bring about gene-specific silencing in the transfected mammalian cells.

g. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present disclosure or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### 5. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regard, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### EXAMPLES

### Example 1: Detection of Advanced Circulating Genetically Abnormal Cells

### Objective

The objective of this study is to determine if certain Circulating Genetically Abnormal Cells (CGAC), parameters such as specific Fluorescent In Situ Hybridization (FISH) Probe Patterns or Nuclear Morphology may be used to identify unique CGACs that are more specific and sensitive for malignant Lung Cancer. These CGACs, referred to herein as Advanced CGACs, may be analyzed and weighed differently than more common CGACs in order to improve overall assay performance.

### Methods

The LungLB test is a 4-color fluorescence in-situ hybridization assay used to identify CGACs isolated from peripheral blood of patients with indeterminate nodules suspicious for lung cancer. Clinical blood samples are drawn by a licensed professional directly into a blood collection tube (ie: Cytochex, CEE-Sure) used to stabilize blood samples for use within 24-96 hours. Stabilized blood samples are initially processed by a highly selective immunomagnetic depletion methodology selectively removing red blood cells and subpopulations of leukocytes using any combination of CD66b, CD14, CD3, CD19, or CD56 antibodies to create an enriched cell suspension.

Enriched cells are deposited on a glass slide followed by FISH using 4-color fluorescent probes targeting regions of the genome known to be amplified in lung cancers (Katz R. L., Zaidi T. M., et al 2008, Katz R. L., He W., et al 2010). Images of the entire slide are acquired using a Bioview wide-field fluorescent microscope and processed using a custom designed, artificial intelligent (AI)-derived algorithm to categorize (classify) CGACs, defined as amplifications of two or more probe regions, which are subsequently verified by a trained and qualified technologist.

Once analyzed, the final research file data is exported from the Bioview system to a CGAC Analysis Database (SQL). The CGAC Analysis Database contains clinical information from each sample as well as analytical data down to each individual CGAC. LungLB results are identified as negative or positive based on an established threshold of CGACs per ten thousand total cells.

### CGAC Probe Patterns

The goal is to identify specific LungLB CGAC subtypes with unique probe patterns that may suggest higher clinical significance and specificity compared to more commonly found CGAC probe patterns. Once identified, determine how weighing the various CTC subtypes differently affects LungLB Assay Sensitivity, Specificity, & AUC.

Study objectives include: Identify CGAC Subtypes with Unique Probe Patterns; Determine Clinical Significance of CGAC Subtypes (Malignancy Rate, ImmunoFISH); Determine Prevalence in True/False Positive & True/False Negative Results; and Identify Which Advanced CGAC Subtypes have the Highest Clinical Significance.

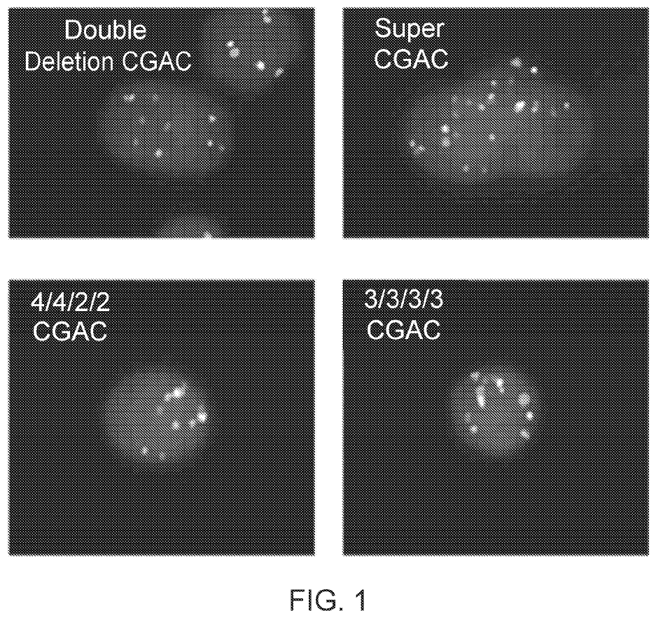

First, CGAC subtypes were identified using LungLB according to their unique probe patterns. Table 1 details Advanced CGAC subtypes and classification/pattern formulas. FIG. 1.

TABLE 1
Advanced CGAC subtypes

| Advanced CGAC Subtypes | Probe Pattern Formula |
|---|---|
| Double Deletion | Gains in 2 Channels + |
| | Deletions in 2 Channels |
| Super CGAC | 5 or More Probes in 2 Channels |
| 4 × 2 | 4Red/4Green OR 4Gold/4Aqua |
| 3/3/3/3 | 3 Probes in All Channels |

Next, the clinical significance of the various CGAC subtypes was examined. The Malignancy Rate was determined and is defined as the percentage of patients with an advanced CGAC subtype that are positive for lung cancer: Malignancy Rate=(Malignant Patients/Total Patients)×100.

TABLE 2
Malignancy rate of advanced CGAC subtypes

| Advanced | Probe Pattern Formula | Total | Benign | Malignant | Malignancy | |
|---|---|---|---|---|---|---|
| CGAC Subtype | Logic Statement | Patients | Patients | Patients | Rate | p-value |
| Double | Gains in 2 Channels + | 27 | 1 | 26 | 96.3% | 0.0028 |
| Deletion | Deletions in 2 Channels | | | | | |
| Super CGAC | 5 or More Probes in 2 | 8 | 1 | 7 | 87.5% | |
| | Channels | | | | | |
| 4 × 2 | 4Red/4Green OR | 24 | 3 | 21 | 87.5% | |
| | 4Gold/4Aqua | | | | | |
| 3 × 3 | 3 Probes in All Channels | 17 | 2 | 15 | 88.2% | 0.0064 |

Double Deletion CGACs have a very high malignancy rate of 96.3%. Out of 27 total patients with a Double Deletion CGAC, 26 were positive for lung cancer. Patients with Double Deletion CGACs had a significantly higher malignancy rate compared to patients without Double Deletion CGACs (p-value=0.0028). Double Deletion CGACs were also compared to the most commonly observed CGAC probe pattern (3Red/3Green). The malignancy rate of Double Deletion CGACs is also significantly higher than that of the 3Red/3Green CTCs (p-value=0.0064).

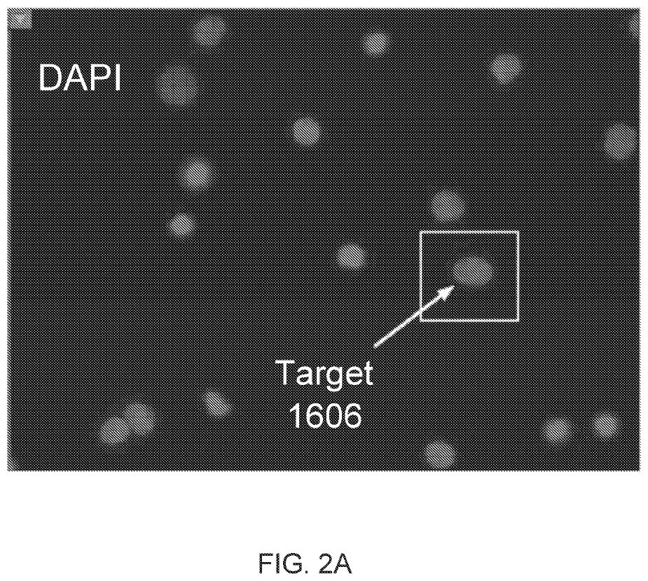

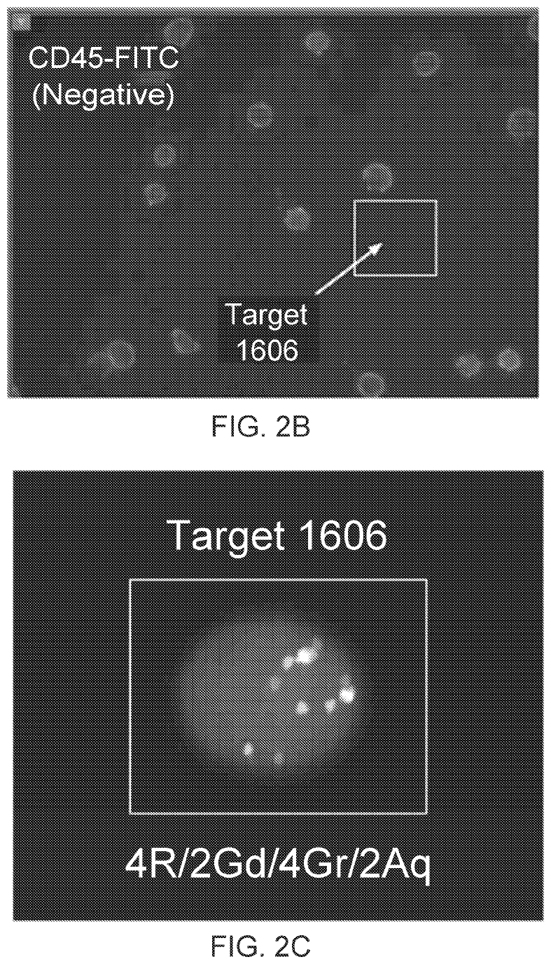

Clinical significance of Advanced CGAC subtypes may also be determined through an ImmunoFISH assay that combines LungLB FISH with immunofluorescent surface markers such as CD45. CD45 is commonly used in industry to differentiate epithelial CGACs from hematopoietic white blood cells. While most cells in the figure below are CD45 Positive, the Advanced CTC Target 1606 with a probe pattern of 4R/2Gd/4Gr/2Aq is CD45 Negative, suggesting higher clinical significance for Lung Cancer. FIG. 2.

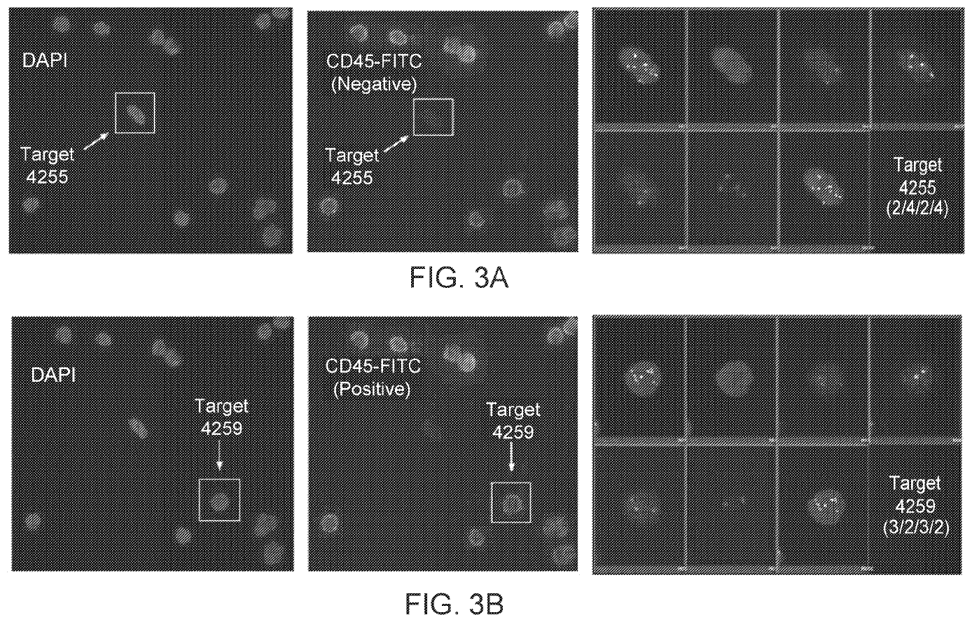

The most common CGAC observed across both benign and malignant LungLB samples have a 3R/2Gd/3Gr/2Aq probe pattern; simply put a single amplification in Red and Green. Therefore, it is important to identify the clinical significance of these cells as well. The figure below depicts CGAC Targets 4255 and 4259 in the same Bioview image. Target 4255 has an Advanced CGAC probe pattern of 2R/4Gd/2Gr/4Aq and is CD45-FITC Negative, suggesting higher clinical significance. Target 4259 has a commonly observed probe pattern of 3R/2Gd/3Gr/2Aq and is CD45-FITC Positive, suggesting this cell is actually a genetically abnormal Leukocyte. FIGS. 3A and 3B.

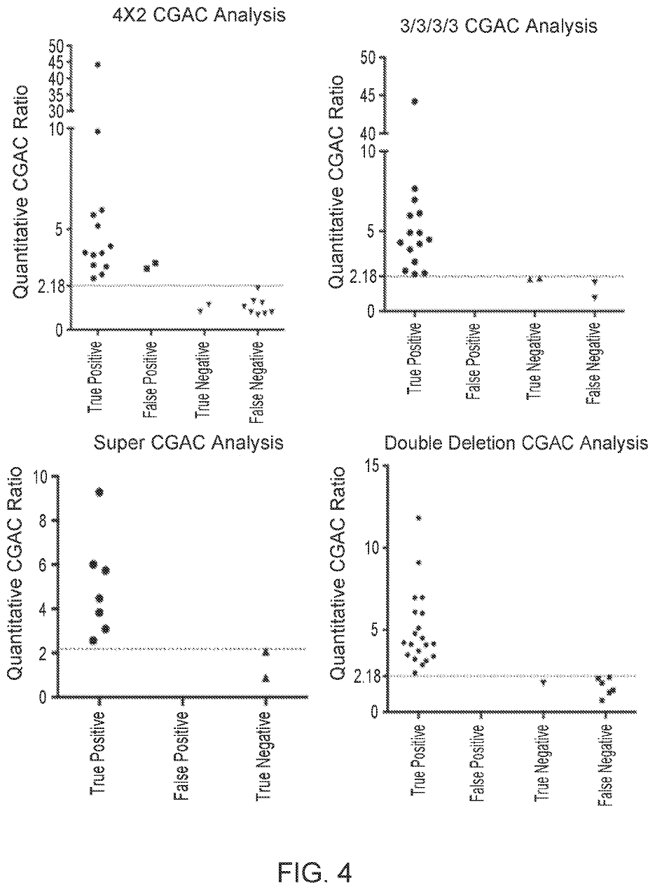

Next, the prevalence of these advanced CGAC subtypes in LungLB samples across True Positive, False Positive, True Negative, and False Negative results is determined. FIG. 4 depicts the prevalence of CGAC Subtypes in patients depending on LungLB assay results. The y-axis represents the Quantitative CGAC Ratio per patient. The dotted line represents the CGAC Ratio Threshold (2.18).

Finally, advanced CGAC subtypes can provide the most predictive value to the LungLB assay were identified. Table 3 demonstrates the prevalence of advanced CGAC subtypes in LungLB True Positives, True Negatives, False Positives, & False Negatives.

TABLE 3
Prevalence of Advanced CGAC subtypes in LungLB assay results.

| CGAC | Total | True | True | False | False |
|---|---|---|---|---|---|
| Subtype | Patients | Positive | Negative | Positive | Negative |
| Double Deletion | 27 | 20 | 1 | 0 | 6 |
| Super CGAC | 8 | 6 | 2 | 0 | 0 |
| 4 × 2 | 24 | 12 | 2 | 2 | 8 |
| 3/3/3/3 | 17 | 13 | 2 | 0 | 2 |

Double Deletion CGAC and 4×2 advanced CGAC offer the highest improvement to the AUC of the LungLB Assay. The number of False Negatives compared to True Negatives provides the clearest picture of Advanced CGAC subtype performance. Weighing these subtypes more heavily has the potential to “flip” incorrect False Negatives into correctly called True Positives. Also, there is the same potential to change correctly called True Negatives into incorrect False Positives.

Therefore, it is determined that Double Deletion and 4×2 CGAC probe patterns should be considered Advanced CGAC patterns, while on the other hand Super CGAC & 3/3/3/3 probe patterns should be considered common CGAC.

TABLE 4
Summary of CGAC and Advanced CGAC prevalence
in LungLB. Advanced CGAC Subtype includes
Double Deletion and 4 × 2 CTCs.

| Circulating | | | | | |
|---|---|---|---|---|---|
| Genetically | 3 Red/3 | 3 Gold/3 | Advance | | |
| Abnormal | Green | Aqua | CGAC | Other | |
| Cells | CGAC | CGAC | Subtype | CGAC | Total |
| CGAC | 1109 | 184 | 65 | 238 | 1596 |
| Count | (69.5%) | (11.5%) | (4.1%) | (14.9%) | (100%) |
| Total | 146 | 101 | 47 | N/A | 151 |
| Subjects | (96.7%) | (66.9%) | (31.1%) | | (100%) |
| With CGAC | | | | | |
| Malignant | 110 | 77 | 43 | N/A | 112 |
| Subjects | (75.3%) | (76.2%) | (91.5%) | | (74.2%) |
| with CGAC | | | | | |
| Benign | 36 | 24 | 4 | N/A | 39 |
| Subjects | (24.7%) | (23.8%) | (8.5%) | | (25.8%) |
| with CGAC | | | | | |

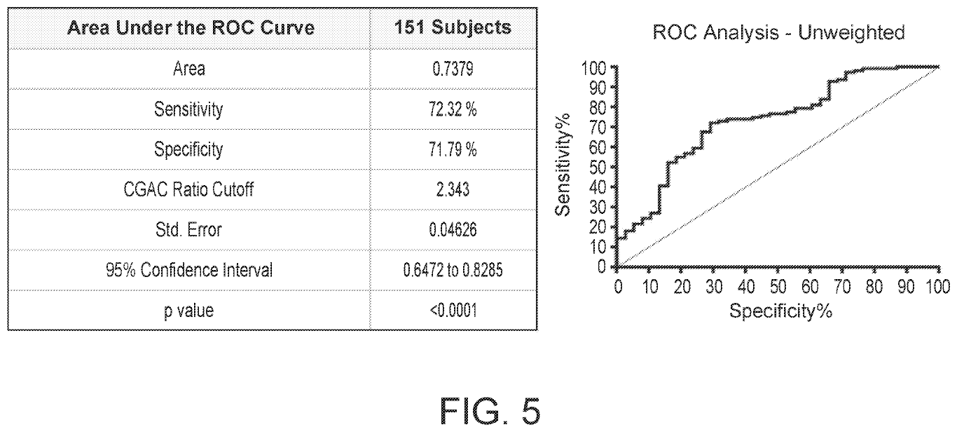

An ROC analysis was performed to assess the overall Sensitivity, Specificity, and AUC of the LungLB assay. An Unweighted ROC analysis was performed first, in which all CTCs were weighed equally, regardless of observed probe patterns (FIG. 5).

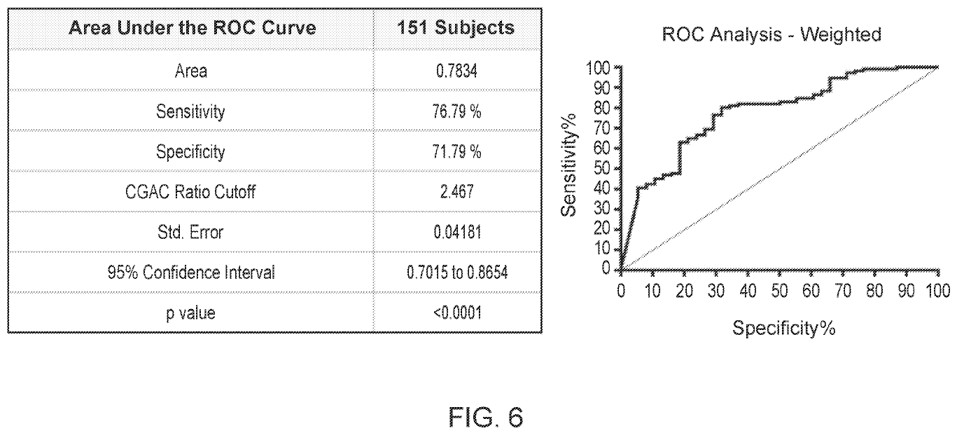

A Weighted ROC analysis was also performed. Advanced CGACs were weighed more heavily in this ROC analysis due to previous data suggesting they are more specific to Lung Cancer compared to typical CGACs. Samples that contained an Advanced CGAC had normalized CGAC ratios automatically turned to 10.0 if the original CGAC ratio was less than 10.0 and declared qualitatively positive. The reason CGAC ratios are set to 10 for ROC analysis is to ensure samples with Advanced CGACs have a positive result throughout the entire ROC curve, regardless of the set threshold (FIG. 6).

### Example 2: CTC Nuclear Morphology

Objective: Determine if nuclear morphology parameters, such as Nuclear Area and Roundness, may be informative or used to distinguish genetically abnormal cells found in benign patients vs. genetically abnormal cells found in malignant patients.

Goals: Establish baseline nuclear area and nuclear roundness parameters for all CGACs from benign and malignant samples (Bioview Duet System); Determine if nuclear morphology parameters may be used to distinguish CGACs found in benign patients vs Advanced CGACs found in malignant patients; Determine if nuclear morphology parameters may be used to distinguish Advanced CGACs subtypes, such as Double Deletion CGACs, in benign patients and malignant patients.

Background: The Bioview analysis software assesses certain nuclear morphology parameters for every cell scanned. Parameters include nuclear area, nuclear roundness, diameter, etc. This data is exported from the Bioview into the CGAC analysis database and may potentially be used to further classify target cells beyond only FISH probe counts.

First, nuclear morphology baselines were established for all CGACs observed in benign and malignant patients: CGAC Nuclear Area Avg. (Benign): 128.3; CGAC Nuclear Area Avg. (Malignant): 126.2; CGAC Nuclear Roundness Avg. (Benign): 1.14; and CGAC Nuclear Roundness Avg. (Malignant): 1.15.

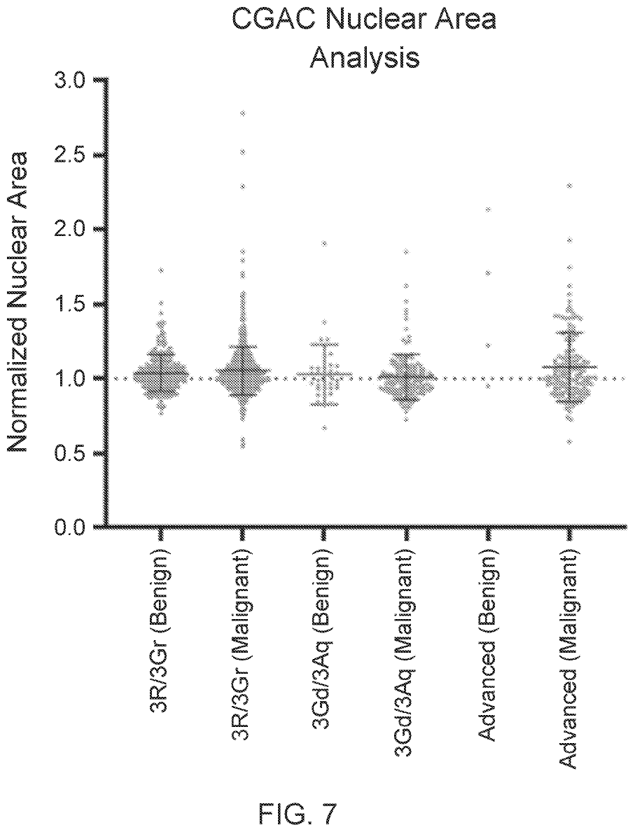

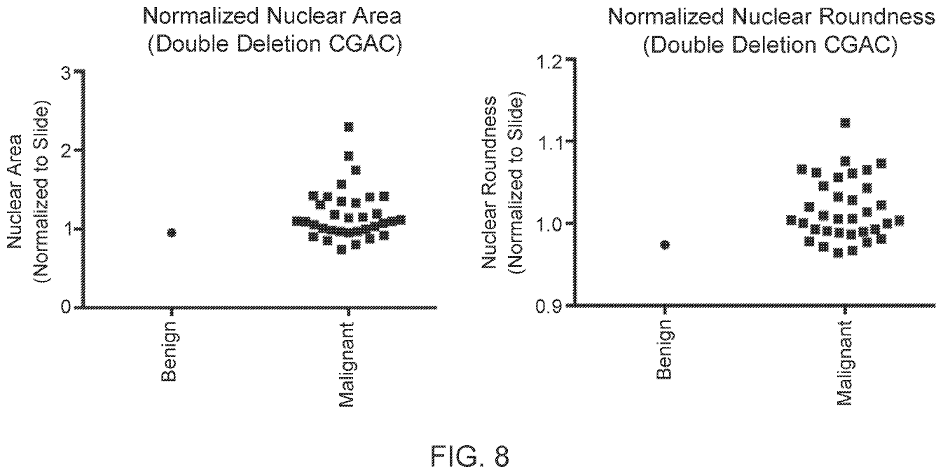

Baseline nuclear morphology parameters between all benign and malignant CTCs were almost identical, suggesting that on the surface there is no distinguishable difference. FIG. 7 visualizes the nuclear area differences of CGACs across benign and malignant patients. There may be some outliers on the higher ends of the scale due to some CGACs observed in clumps, which the Bioview system could misclassify as being very large.

Next, it was determined if nuclear morphology parameters may be used to distinguish CGACs found in benign subjects vs Advanced CGACs found in malignant subjects (Table 5).

TABLE 5
Benign vs Advanced CGAC nuclear Area

3R/3Gr (Benign): 1.04 −> 4% Increase
3R/3Gr (Malignant): 1.05 −> 5% Increase
3Gd/3Aq (Benign): 1.03 −> 3% Increase
3Gd/3Aq (Malignant): 1.01 −> 1% Increase
Advanced CTC (Benign): 1.51 −> 51% Increase*
Advanced CTC (Malignant): 1.19 −> 19% Increase

The most common CGAC probe patterns (3Red/3Green or 3Gold/3Aqua) demonstrated an equivalent Nuclear Area to genetically normal cells, regardless of being observed in benign or malignant subjects. On the other hand, Advanced CGACs from malignant subjects have an almost 20% higher nuclear area on average compared to genetically normal cells. This may prove to be clinically significant in distinguishing between commonly observed CGACs and Advanced CGACs.

Finally, it was determined if any nuclear morphology parameters assessed across CGACs may be applied more specifically to Advanced subtypes such as Double Deletion CGACs to distinguish between malignant and benign samples.

### Example 3: CGAC-level Analysis on Probe Pattern and Morphology

The LungLB test defines a CGAC as a cell enriched from blood using ficoll density centrifugation that shows a gain in two or more channels when using a 4-color FISH assay. CGAC that show different probe patterns between subjects and within the same subject. CGAC identified in subjects with benign pulmonary lesions would suggest a “false positive” signal. If false positive signals can be excluded from analysis it could serve to improve assay performance.

The Bioview microscope software is capable of exporting data on cells into a “comma separated value” format which can be loaded into Excel or database program. Exported data include multiple parameters, including probe patterns and cell morphology, at single-cell resolution. Using these exports, FISH probe patterns and cell morphology attributes were identified which can improve the way in which clinical slides are analyzed.

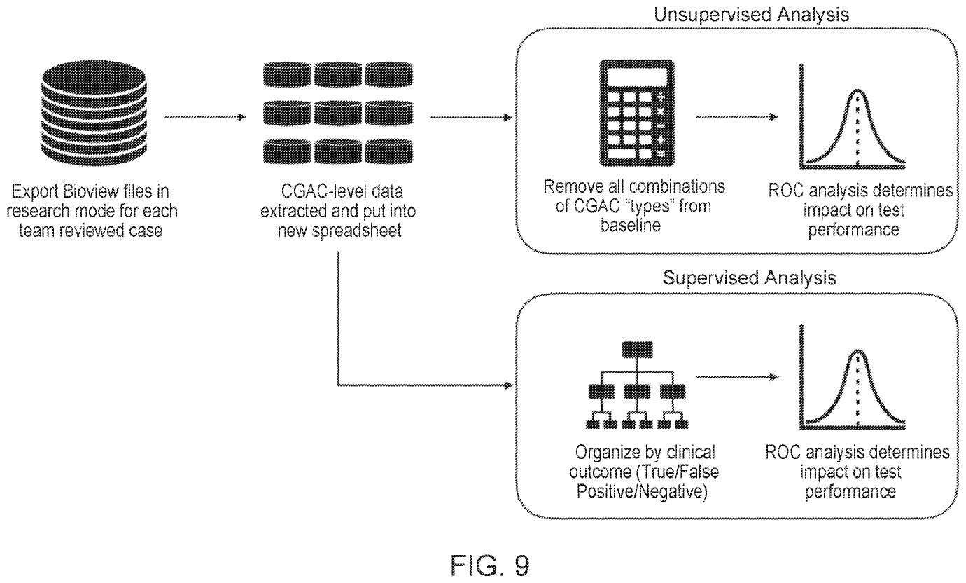

Using bioview software, each CGAC are characterized based on probe patterns (see Table 6 for list of CGAC “types” based on probe pattern). Next, either an unsupervised or supervised analysis will take place. For Unsupervised, previous knowledge of biopsy result will not influence which CGAC types are removed. For supervised, CGAC patterns identified in benign cases (true negative and false positive results) will be analyzed for trends. Finally, an ROC analysis will be performed based on Adjusted CGAC counts (baseline minus each individual and combination of CGAC type) to determine change in test performance (FIG. 9).

TABLE 6
Logic data for CGAC hybridization pattenrs

| Name | Description | Probe Pattern Logic Statements |
|---|---|---|
| Red/Green | Gain in green and red channels only | green >2 AND red >2 AND |
| | | aqua/gold = 2 |
| | | red, green >2 AND aqua = 2, gold <2 OR |
| | | aqua <2, gold = 2 (single deletion) |
| Aqua/Gold | Gain in aqua and gold channels only | aqua >2 AND gold >2 AND |
| | | red/green = 2 |
| | | aqua/gold >2 AND red = 2, green <2 OR |
| | | red <2, green = 2 (single deletion) |
| Mismatch | Gains in mis-matched chromosomes (i.e. | aqua, green >2, red = 2, gold ≤2 |
| | Aqua/Green) | aqua, green >2, red ≤2, gold = 2 |
| | Green and Red are on chromosome 3. | aqua, red >2, green = 2, gold ≤2 |
| | Aqua and gold are on chromosome 10. | aqua, red >2, green ≤2, gold = 2 |
| | | gold, green >2, red = 2, aqua ≤2 |
| | | gold, green >2, red ≤2, aqua = 2 |
| | | gold, red >2, aqua = 2, green ≤2 |
| | | gold, red >2, aqua ≤2, green = 2 |
| Double | Simultaneous gain in two channels and | green/red >2, aqua/gold <2 |
| Deletion | deletion in two channels | green/red <2, aqua/gold >2 |
| | | aqua/red >2, green/gold <2 |
| | | aqua/red <2, green/gold >2 |
| | | aqua/green >2, red/gold <2 |
| | | aqua/green <2, red/gold >2 |
| Triple | Gain in three channels only | green, red, aqua >2, gold ≤2 |
| | Note: should do a secondary analysis | green, red, gold >2, aqua ≤2 |
| | where these are considered green/red or | aqua, gold, red >2, green ≤2 |
| | aqua/gold | aqua, gold, green >2, red ≤2 |
| Both | Gain in both Green/Red and Aqua/Gold | green AND red AND aqua AND |
| | pairs | gold >2 |

Preliminary analysis of the data show that ˜70 of all CGAC identified fall into the green/red category. Therefore, it is unlikely that removal of CGAC with this probe pattern will have a positive impact on test performance. As such, for the unsupervised analysis there are 5 CGAC “categories” (Aqua/gold, mismatch, double deletion, triple, and both) that will be removed in all possible combinations. There are 31 possible unique combinations of those 5 CTC categories described in the equation below:

$${\sum }_{k = 1}^{n} \frac{n !}{k ! ( n - k ) !}$$

Where “n” is the number of CGAC categories under consideration (5 in this case, as Red/Green will not be included) and “k” is the number of categories being considered at a time (i.e. removal of 1 CGAC category versus 2 CGAC categories, etc).

### Results

### Probe Level Analysis

Data was exported from Bioview in research mode. CGAC-level data were extracted using MySQL scripts, exported into Excel and each CTC was categorized based on the logic data shown in Table 6. Step 0 is CGAC level data for all 1016 CTC identified across each slide in the analysis. Step 1 shows slide-level data for all 217 Bioview files that were analyzed. Basic statistics from these data are shown in Table 7.

TABLE 7
CGAC Class Statistics

| | Red/Green | Aqua/Gold | Mismatch | Double Deletion | Both | Triple | Total |
|---|---|---|---|---|---|---|---|
| CGAC Count | 725 | 117 | 74 | 18 | 41 | 41 | 1016 |
| % of CGAC | 71.4% | 11.5% | 7.3% | 1.8% | 4.0% | 4.0% | 100% |
| # of cases | 91 | 62 | 31 | 14 | 28 | 28 | 95 |
| % of cases | 95.8% | 65.3% | 32.6% | 14.7% | 29.5% | 29.5% | 100% |
| Unique Cases | 11 | 1 | 1 | 0 | 0 | 0 | |

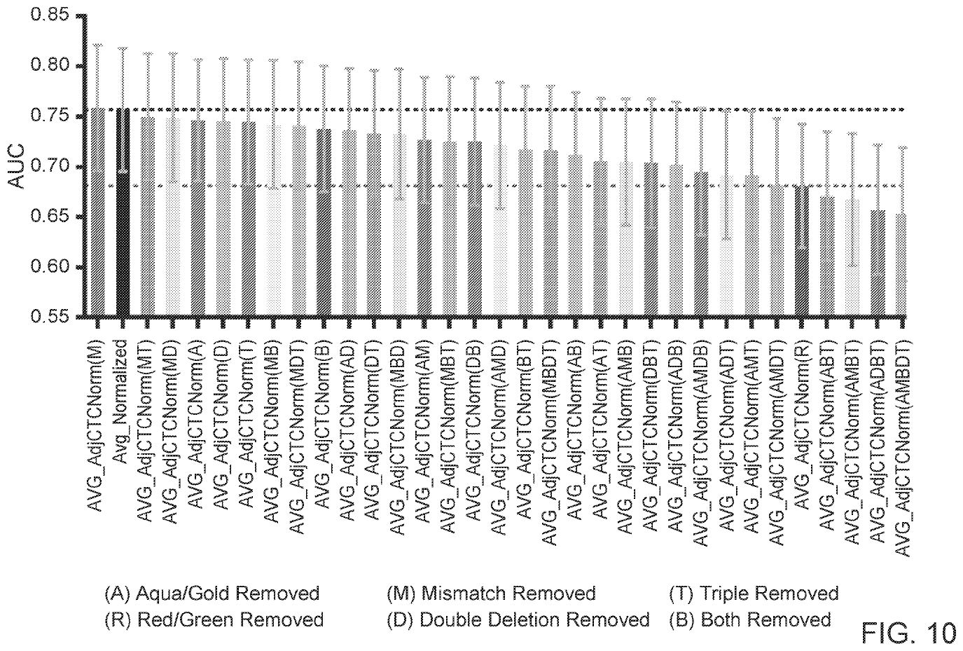

Step 2 calculates the normalized CGAC count for each file (CGAC count/Total cell count*10,000). An adjusted CTC count (Original count−CGAC class count) and an adjusted count that has been normalized were calculated. It has been determined that taking the average of the two closest normalized CGAC count yields the best test performance. As such, Step 3 takes the average of the two closest values for each of the 31 possible combinations of CGAC classes that were removed. These values were then placed into three columns based on the biopsy result (benign, malignant, indeterminate) and a Receiver Operator Characteristics (ROC) curve was generated and Area Under the Curve (AUC) calculated (FIG. 10).

The baseline result with no CGAC removed shows an AUC of 0.7568. Because the Red/Green CGAC class (R) is represented in >95% of all cases it was used as a negative control as removal of those CGAC should have a detrimental impact on test performance. Only removal of the Mismatch CGAC class resulted in a modest increase in AUC to 0.7587; however, using 1-way ANOVA and multiple comparisons no dataset showed a statistical difference compared to baseline.

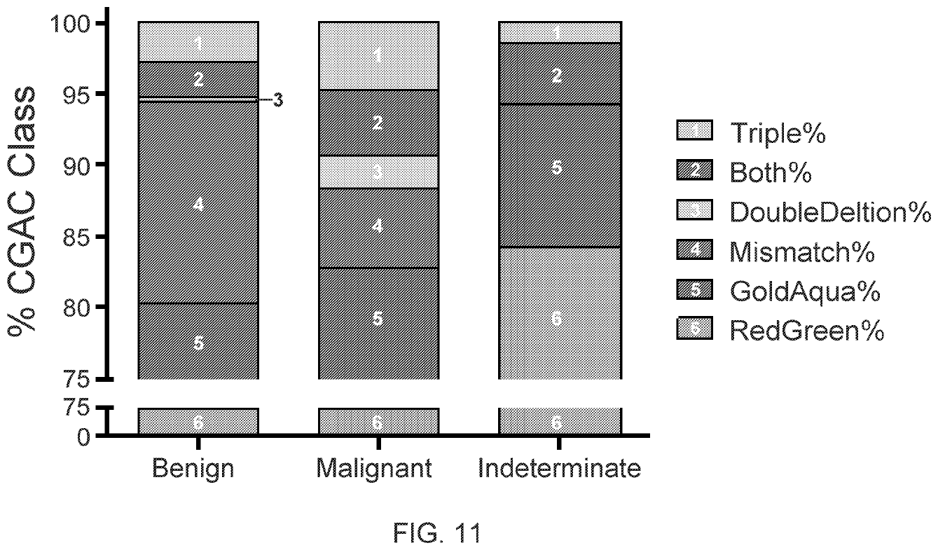

In order to determine the frequency by which each CGAC class are found in subjects with malignant and benign lesions, CGAC from each subject were sorted by biopsy result. FIG. 11 highlights that Mismatch CGAC are more often found in subjects with benign lesions (14.1% vs 5.6%, P<0.0001 Chi-square test), which is consistent the finding that removal of this CGAC type increased the AUC compared to baseline. Not obvious in the unsupervised analysis is that Double Deletion, Both, and Triple CGAC are more often found in subjects with malignant lesions (2.4% vs 0.4%, 4.5% vs 2.4%, and 4.7% vs 2.8%, respectively)

Probe-level analysis using both unsupervised and supervised analyses suggest that “Mismatch” CGAC are more commonly associated with benign nodules. Excluding this class of CGAC from analysis may increase test specificity. The Double Deletion class is more often found in subjects with malignant nodules. If Mismatch and Double Deletion CGAC continue to trend with benign and malignant nodules, it may be worth considering having them excluded or weighted more heavily during analysis, respectively.

### Example 4: CGAC Analysis Patient Level

A step-wise logistic regression was performed using >1700 CGAC categorized by FISH probe pattern in order to determine the relative independent and combined contributions of each CGAC subtype at predicting malignancy.

The data suggest that the presence or absence (dichotomous result) of individual probes are not predictive (green, red, aqua, gold). Prescence of advanced CGAC were predictive (combo of double del, 4×2, Super CTC), as was double deletion on its own. Nuclear parameters (area, roundness, diameter) were not individually predictive. Prescence of cells with 3 red/3 green or 3 aq/3 gold probe patters were also not predictive (this is because we see them in both benign and malignant patients and we know it's the number of cells that is predictive).

Each predictor below was entered into a logistic regression model with diagnosis as outcome. Cluster robust variance estimators were used to account for within-patient correlation.

TABLE 8

| Predictor | OR (95% CI) | P-value |
|---|---|---|
| Green | 1.03 (0.73, 1.45) | 0.853 |
| Red | 1.26 (0.96, 1.66) | 0.101 |
| Aqua | 0.95 (0.74, 1.23) | 0.72 |
| Gold | 1.03 (0.64, 1.68) | 0.898 |
| Advanced CGAC | 4.35 (1.39, 13.62) | 0.011 |
| Double Deletion | 9.45 (1.14, 78.74) | 0.038 |
| Super CGAC | 3.38 (0.38, 29.91) | 0.273 |
| 4 by 2 CGAC | 2.55 (0.72, 9.07) | 0.147 |
| 3333 CGAC | 2.54 (0.52, 12.38) | 0.248 |
| Area - Normalized | 1.53 (0.63, 3.72) | 0.351 |
| Round - Normalized | 98.45 (0.82, >999) | 0.06 |
| Diameter - Normalized | 3.06 (0.51, 18.23) | 0.22 |
| 3 Red/3 Green | 0.92 (0.65, 1.29) | 0.62 |
| 3 Aqua/3 Gold | 1.23 (0.75, 2.02) | 0.42 |

Each of the following interactions below was tested for significance in its own model.

TABLE 9

| Interaction | P-value |
|---|---|
| Double Deletion × Round Normalized | 0.003 |
| Double Deletion × Area Normalized | 0.004 |
| Double Deletion × Diameter Normalized | 0.005 |
| Red × Round Normalized | 0.63 |
| Red × Area Normalized | 0.88 |
| Red × Diameter Normalized | 0.97 |
| 3 Red/3 Green × Round Normalized | 0.77 |
| 3 Red/3 Green × Area Normalized | 0.88 |
| 3 Red/3 Green × Diameter Normalized | 0.99 |
| 3 Aqua/3 Gold × Round Normalized | 0.55 |
| 3 Aqua/3 Gold × Area Normalized | 0.41 |
| 3 Aqua/3 Gold × Diameter Normalized | 0.46 |

Univariate models or subgroups of interest. Subgroup: 3 Red/3 Green.

TABLE 10

| Predictor | OR (95% CI) | P-value |
|---|---|---|
| Area - Normalized | 1.48 (0.59, 3.7) | 0.404 |
| Round - Normalized | >99 (0.61, >999) | 0.075 |
| Diameter - Normalized | 3.04 (0.46, 19.97) | 0.248 |

Subgroup: 3 Aqua/3 Gold

TABLE 11

| Predictor | OR (95% CI) | P-value |
|---|---|---|
| Area - Normalized | 0.56 (0.04, 7.47) | 0.658 |
| Round - Normalized | >99 (0, >999) | 0.327 |
| Diameter - Normalized | 0.47 (0, 103.45) | 0.782 |

Analyzed probe patterns include those in table 12.

TABLE 12
Probe patterns analyzed

| | Benign | | Malignant | |
|---|---|---|---|---|
| | (N = 370) | | (N = 1321) | |
| | N | % | N | % |
| 0 green, 0 red, 3 aqua, 3 gold | 1 | 0.27 | 2 | 0.15 |
| 0 green, 0 red, 4 aqua, 4 gold | 0 | 0 | 1 | 0.08 |
| 1 green, 1 red, 3 aqua, 3 gold | 0 | 0 | 7 | 0.53 |
| 1 green, 1 red, 4 aqua, 3 gold | 0 | 0 | 1 | 0.08 |
| 1 green, 1 red, 4 aqua, 4 gold | 0 | 0 | 1 | 0.08 |
| 1 green, 1 red, 5 aqua, 5 gold | 0 | 0 | 1 | 0.08 |
| 1 green, 3 red, 3 aqua, 2 gold | 0 | 0 | 3 | 0.23 |
| 2 green, 1 red, 3 aqua, 3 gold | 0 | 0 | 1 | 0.08 |
| 2 green, 2 red, 3 aqua, 3 gold | 35 | 9.46 | 147 | 11.13 |
| 2 green, 2 red, 3 aqua, 4 gold | 1 | 0.27 | 1 | 0.08 |
| 2 green, 2 red, 4 aqua, 3 gold | 1 | 0.27 | 2 | 0.15 |
| 2 green, 2 red, 4 aqua, 4 gold | 2 | 0.54 | 9 | 0.68 |
| 2 green, 3 red, 2 aqua, 3 gold | 3 | 0.81 | 3 | 0.23 |
| 2 green, 3 red, 3 aqua, 2 gold | 3 | 0.81 | 21 | 1.59 |
| 2 green, 3 red, 3 aqua, 3 gold | 2 | 0.54 | 2 | 0.15 |
| 2 green, 3 red, 4 aqua, 4 gold | 0 | 0 | 1 | 0.08 |
| 2 green, 3 red, 5 aqua, 4 gold | 1 | 0.27 | 0 | 0 |
| 3 green, 2 red, 2 aqua, 3 gold | 6 | 1.62 | 8 | 0.61 |
| 3 green, 2 red, 3 aqua, 2 gold | 19 | 5.14 | 7 | 0.53 |
| 3 green, 2 red, 3 aqua, 3 gold | 1 | 0.27 | 4 | 0.3 |
| 3 green, 2 red, 4 aqua, 3 gold | 0 | 0 | 1 | 0.08 |
| 3 green, 2 red, 4 aqua, 4 gold | 0 | 0 | 1 | 0.08 |
| 3 green, 3 red, 0 aqua, 0 gold | 0 | 0 | 3 | 0.23 |
| 3 green, 3 red, 0 aqua, 2 gold | 0 | 0 | 1 | 0.08 |
| 3 green, 3 red, 1 aqua, 1 gold | 0 | 0 | 9 | 0.68 |
| 3 green, 3 red, 1 aqua, 2 gold | 6 | 1.62 | 24 | 1.82 |
| 3 green, 3 red, 1 aqua, 3 gold | 1 | 0.27 | 1 | 0.08 |
| 3 green, 3 red, 2 aqua, 1 gold | 0 | 0 | 2 | 0.15 |
| 3 green, 3 red, 2 aqua, 2 gold | 263 | 71.08 | 915 | 69.27 |
| 3 green, 3 red, 2 aqua, 3 gold | 1 | 0.27 | 12 | 0.91 |
| 3 green, 3 red, 3 aqua, 2 gold | 4 | 1.08 | 13 | 0.98 |
| 3 green, 3 red, 3 aqua, 3 gold | 2 | 0.54 | 18 | 1.36 |
| 3 green, 3 red, 3 aqua, 4 gold | 1 | 0.27 | 0 | 0 |
| 3 green, 3 red, 4 aqua, 1 gold | 0 | 0 | 1 | 0.08 |
| 3 green, 3 red, 4 aqua, 3 gold | 1 | 0.27 | 3 | 0.23 |
| 3 green, 3 red, 4 aqua, 4 gold | 2 | 0.54 | 7 | 0.53 |
| 3 green, 4 red, 2 aqua, 2 gold | 3 | 0.81 | 3 | 0.23 |
| 3 green, 4 red, 2 aqua, 3 gold | 0 | 0 | 1 | 0.08 |
| 3 green, 4 red, 3 aqua, 2 gold | 0 | 0 | 2 | 0.15 |
| 3 green, 4 red, 3 aqua, 3 gold | 0 | 0 | 4 | 0.3 |
| 3 green, 4 red, 3 aqua, 4 gold | 0 | 0 | 1 | 0.08 |
| 4 green, 2 red, 2 aqua, 3 gold | 0 | 0 | 2 | 0.15 |
| 4 green, 2 red, 3 aqua, 2 gold | 1 | 0.27 | 0 | 0 |
| 4 green, 2 red, 4 aqua, 3 gold | 0 | 0 | 1 | 0.08 |
| 4 green, 3 red, 2 aqua, 2 gold | 2 | 0.54 | 2 | 0.15 |
| 4 green, 3 red, 3 aqua, 2 gold | 0 | 0 | 1 | 0.08 |
| 4 green, 3 red, 3 aqua, 3 gold | 0 | 0 | 1 | 0.08 |
| 4 green, 3 red, 4 aqua, 2 gold | 0 | 0 | 1 | 0.08 |
| 4 green, 3 red, 6 aqua, 7 gold | 0 | 0 | 1 | 0.08 |
| 4 green, 4 red, 0 aqua, 0 gold | 0 | 0 | 1 | 0.08 |
| 4 green, 4 red, 1 aqua, 1 gold | 0 | 0 | 3 | 0.23 |
| 4 green, 4 red, 2 aqua, 2 gold | 1 | 0.27 | 18 | 1.36 |
| 4 green, 4 red, 2 aqua, 3 gold | 0 | 0 | 1 | 0.08 |
| 4 green, 4 red, 3 aqua, 2 gold | 0 | 0 | 2 | 0.15 |
| 4 green, 4 red, 3 aqua, 3 gold | 6 | 1.62 | 32 | 2.42 |
| 4 green, 4 red, 5 aqua, 5 gold | 0 | 0 | 1 | 0.08 |
| 5 green, 4 red, 3 aqua, 3 gold | 0 | 0 | 1 | 0.08 |
| 5 green, 5 red, 0 aqua, 0 gold | 0 | 0 | 1 | 0.08 |
| 5 green, 5 red, 1 aqua, 1 gold | 0 | 0 | 2 | 0.15 |
| 5 green, 5 red, 2 aqua, 2 gold | 0 | 0 | 1 | 0.08 |
| 5 green, 5 red, 3 aqua, 3 gold | 0 | 0 | 1 | 0.08 |
| 5 green, 5 red, 3 aqua, 4 gold | 0 | 0 | 1 | 0.08 |
| 5 green, 5 red, 5 aqua, 5 gold | 1 | 0.27 | 0 | 0 |
| 6 green, 6 red, 3 aqua, 3 gold | 0 | 0 | 1 | 0.08 |
| 7 green, 7 red, 0 aqua, 0 gold | 0 | 0 | 1 | 0.08 |
| 10 green, 10 red, 3 aqua, 3 gold | 0 | 0 | 1 | 0.08 |

### Example 5: Presence of Circulating Genetically Abnormal Cells in the Peripheral Blood of Individuals with Indeterminate Pulmonary Nodules can be Used to Accurately Detect Lung Cancer

Computed tomography is the standard method by which pulmonary nodules are detected. Radiological characteristics and clinical risk assessments guide clinicians on when a biopsy is indicated. However, >40% of biopsies of pulmonary nodules, which often lead to complications, are not lung cancer and therefore unnecessary. The LungLB™ test was developed to aid in the clinical assessment of patients with indeterminate nodules suspicious for lung cancer. The test is based on findings suggesting that circulating genetically abnormal cells (CGACs) are present early in lung cancer pathogenesis.

### Materials and Methods

### Participant Enrollment

Participants for this study were enrolled from MD Anderson Cancer Center in Houston, Texas and the Mount Sinai Health System in New York, New York. Physicians, participants, and laboratory and statistical personnel were blinded to the results of the test and clinical information. The blinding protocol was strictly followed, and the results of the test did not direct or influence participant care. All sites had institutional review board approval, and informed written consent was obtained from all eligible participants.

Eligible participants were older than 18 years of age and scheduled for percutaneous needle biopsy. There were no restrictions on nodule characteristics to avoid bias from radiological factors. Participants were ineligible if they had a prior or concurrent cancer diagnosis of any type, or a lung cancer diagnosis within the past 2 years. Inclusion and exclusion criteria were intentionally kept broad to avoid bias and reflect real-world conditions.

### Peripheral Blood Collection

Peripheral blood was collected just prior to the CT-guided needle biopsy procedure. Blood was collected in vacutainer tubes containing blood stabilizer (Streck, Omaha, NE) and shipped overnight to LungLife AI's Clinical Laboratory Improvement Amendments (CLIA)-certified lab in Thousand Oaks, CA.

### CGAC Enrichment

Samples received by the CLIA-certified lab were accessioned into LungLife AI's laboratory information management system using 2 unique identifiers. Blood was centrifuged at 700×g for 10 minutes with the brake off. Plasma was transferred to new tubes and stored at −80° C. Erythrocytes were removed using an ammonium chloride-based erythrocyte lysis buffer. The remaining leukocytes were quantified using a BD Accuri™ C6 flow cytometer (Becton Dickenson, San Jose, CA) and 5,000,000 leukocytes were transferred to a new tube for magnetic cell depletion. Cells were incubated with biotinylated antibodies targeting CD66b and CD14 (BioLegend, San Diego, CA) for removal of neutrophils and monocytes, respectively. This was followed by incubation with paramagnetic streptavidin coated particles (BD Biosciences, San Jose, CA) and subsequent magnetic separation, and the supernatant was transferred to a new tube.

### Cell Cryopreservation and Thawing

Leukocytes not used in the depletion procedure were prepared for cryopreservation. Cells were resuspended in 1 mL cryopreservation medium containing 10% DMSO and slowly frozen in a −80° C. freezer (−1° C./min) and then transferred to liquid nitrogen. Cells were thawed in a 37° C. water bath for approximately 2 minutes, followed by 2 washes with 10 mL PBS containing 10% FBS to reduce DMSO.

### Fluorescence In Situ Hybridization

Ten thousand (10,000) cells from the cell suspension were then transferred to a glass slide using a cytospin instrument. Cells were fixed in Carnoy's fixative (3:1 solution of methanol and glacial acetic acid) for 30 minutes, followed by treatment with protease (pepsin pH 2, Abbot Molecular, Abbott Park, IL). Four colored FISH probes described elsewhere (Katz R L et al, 2020) were then added to the microscope slide and a coverslip was affixed using rubber cement. DNA was denatured at 80° C. for 2 minutes, followed by overnight hybridization in a humidified chamber for 18 hours. Slides were then washed, and a new coverslip was applied with mounting medium containing 4′,6-diamidino-2-phenylindole (DAPI; Vector Labs, Burlingame, CA) to visualize cell nuclei.

### Image Acquisition and Analysis

Slides containing cells were imaged using a Bioview Allegro-Plus microscope system (Bioview USA, Billerica, MA). Images were acquired using a 60× objective (1.35 NA oil immersion on UPlanSapo, Olympus, Bartlett, TN) and a FLIR Grasshopper 3 monochrome camera (12-bit, 2448×2048 pixels, 3.4 μm pixel size, Edmund, Barrington, NJ) controlled using Bioview Duet software. All cells were imaged with 21 transverse sections spanning 0.65 μm.

Objects were classified by the Bioview Duet software according to probe copy number variation. Normal cells have 2 spots in all 4 color channels (Red, Green, Aqua, and Gold) and CGACs have a gain of spots in ≥2 color channels. Advanced CGACs were CGACs with the following specific anomalies: 4 spots in 2 color channels (4×2 Advanced CGAC), or a gain of spots in 2 color channels plus any loss of spots in 2 color channels (Double Deletion Advanced CGAC). Though Advanced CGACs can be considered a subtype of CGACs, classification of cells as a CGAC or Advanced CGAC was mutually exclusive in this study. Cells binned in the CGAC classes by the BioView Duet software were analyzed by a licensed technician who verified each cell. CGAC counts were normalized by dividing the CGAC count by the total number of cells analyzed and multiplying by 10,000. A minimum of 10,000 cells were analyzed per participant. Total CGAC count, total cell count, and normalized CGAC counts were sent for unblinding for each participant.

### Statistics

The statistical significance of clinical factor data was determined using the Mann-Whitney test (two-tailed, 95% confidence interval), Fisher's Exact test (two-tailed, 95% confidence interval), and Chi-Square test (two-tailed, 95% confidence interval). To determine the significance of different CGAC subtypes between participants with malignant and benign diagnosis, the Fisher's Exact Test (two-tailed, 95% confidence interval) or Chi-Square test (two-tailed, 95% confidence interval) were used depending on the sample size. A P<0.05 was considered statistically significant in all analyses.

The nuclear area of cells was measured from DAPI stained cells using the Bioview software and data was exported to Prism (GraphPad Prism 9.3.0, San Diego, CA) for analysis of descriptive statistics.

To establish sensitivity, specificity, and area under the curve (AUC) for the LungLB™ test, a threshold of 2.47 CGAC cells per 10,000 cells was defined. Receiver operating characteristic (ROC) analysis was performed in Prism using normalized CGAC counts from case and control participants (participants with malignant and benign nodules, respectively). For ROC analyses that were weighted for Advanced CGACs, the normalized CGAC ratio for samples containing Advanced CGACs was automatically set to 10.0 and declared qualitatively positive, regardless of the original CGAC ratio.

For the multivariate analysis, the predictors of interest were entered into a univariate and multivariate logistic regression model with cancer diagnosis as the outcome. The AUCs were compared using DeLong tests (two-sided). Complete case analysis was performed, and all analyses were completed in R v4.1.0 (R Foundation for Statistical Computing, Vienna, Austria).

### Results

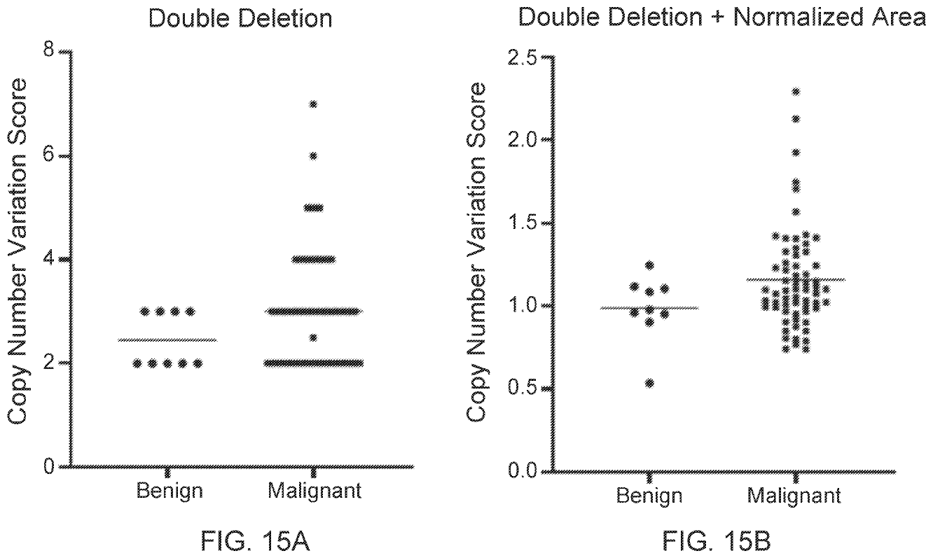

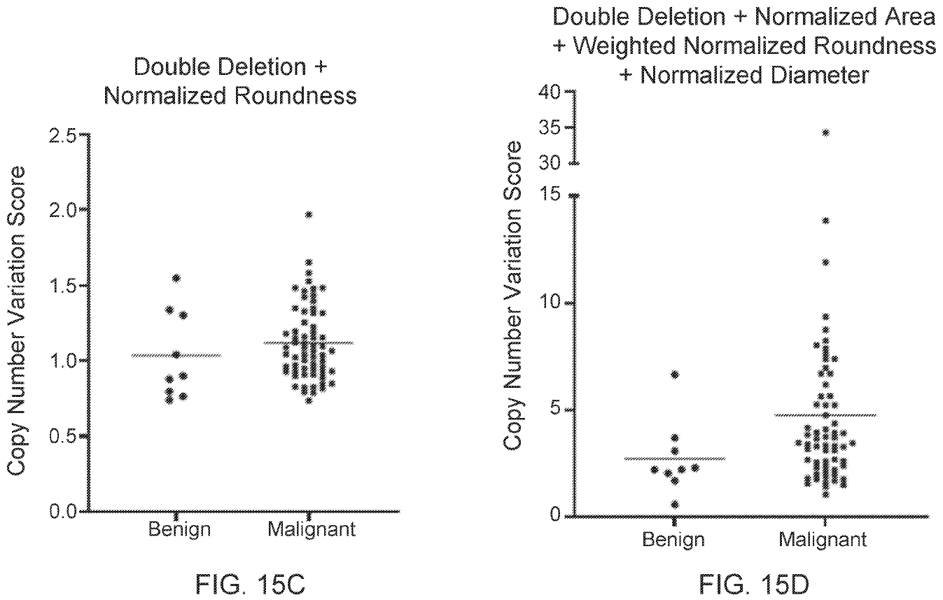

Between December 2018 and February 2021, 182 participants were assessed for eligibility for the study; 19 participants were excluded due to an indeterminate biopsy result (n=14) or having a sample that was unable to be processed due to clotting or damage (n=5) FIG. 15). Samples from 12 of the 161 participants who met the initial eligibility criteria did not pass the assay quality control and these participants were excluded. A total of 151 participants met all study inclusion criteria and were included in the final analysis. Of these 151 participants, 112 participants were diagnosed with malignant lung lesions and 39 participants with benign lung lesions based on blinded nodule biopsy results. After unblinding, participant and disease characteristics commonly used in malignancy prediction models (eg, participant age, smoking history, nodule size, etc) were compared in participants with benign versus malignant nodules and no statistically significant differences were found (Table 13), indicating there was no demonstrable participant selection bias. The clinical risk model developed by the Mayo Clinic was utilized, which is the most validated and commonly used model for predicting the probability of malignancy in suspicious pulmonary nodules, to further characterize the participants in the instant study. With application of the Mayo Clinic Model, the majority of participants (107 [70.8%] of 151 participants) in the instant study fell into the intermediate-risk category as defined by ACCP guidelines (pre-test probability of malignancy between 5%-65%), for which current evaluation and management guidelines are not well-standardized and represents the most challenging diagnostic group.

TABLE 13
Participant Characteristics

| | Overall (N = 151) | | |
|---|---|---|---|
| | Malignant | Benign | |
| | Nodules | Nodules | |
| Category, n (%) | (n = 112) | (n = 39) | P value |
| Sample n/N, % | 74.2 | 25.8 | N/A |
| Gender | | | |
| Male | 52 (46.4) | 17 (43.6) | .8525* |
| Female | 60 (53.6) | 22 (56.4) | |
| Median age, y | 70.0 | 71.0 | .7838^{†} |
| Smoking Status | | | |
| Current | 18 (11.9) | 6 (4.0) | .9115^{§} |
| Former | 70 (46.4) | 26 (17.2) | |
| Never | 23 (15.2) | 7 (4.6) | |
| Unknown | 1 (0.7) | 0 (0) | |
| Median Pack Years, y | 25.0 | 22.8 | .9083^{†} |
| Nodule Size | | | |
| <1 cm | 16 (14.3) | 13 (33.3) | .0654^{§} |
| 1 cm to <2 cm | 41 (36.6) | 13 (33.3) | |
| 2 cm to <3 cm | 32 (28.6) | 7 (18.0) | |
| ≥3 cm | 23 (20.5) | 6 (15.4) | |
| Malignant Lesion Type | | | |
| Adenocarcinoma | 73 (65.2) | — | |
| Squamous Cell Carcinoma | 17 (15.2) | — | |
| Small Cell and Neuroendocrine | 17 (15.2) | — | |
| Other | 5 (4.6) | — | |
| Cancer Stage | | | |
| I | 67 (59.8) | — | |
| II | 9 (8.0) | — | |
| III | 6 (5.4) | — | |
| IV | 6 (5.4) | — | |
| Unknown | 24 (21.4) | — | |
| Benign Lesion Type | | | |
| Infectious Etiology | | 8 (20.5) | |
| Non-infectious Inflammation | | 7 (17.9) | |
| Hamartoma | | 3 (7.7) | |
| Atypical Cells | | 5 (12.8) | |
| Other | | 16 (41.0) | |
| Risk Stratification Based on | | | .0027^{§} |
| Mayo Clinic Model | | | |
| Low Risk (>5%) | 17 (15.2) | 3 (7.7) | |
| Intermediate Risk (5%-65%) | 74 (66.1) | 33 (84.6) | |
| High Risk (<65%) | 21 (18.7) | 3 (7.7) | |

^{a} Fisher's Exact.
^{b} Mann-Whitney.
^{c} Chi-square test.

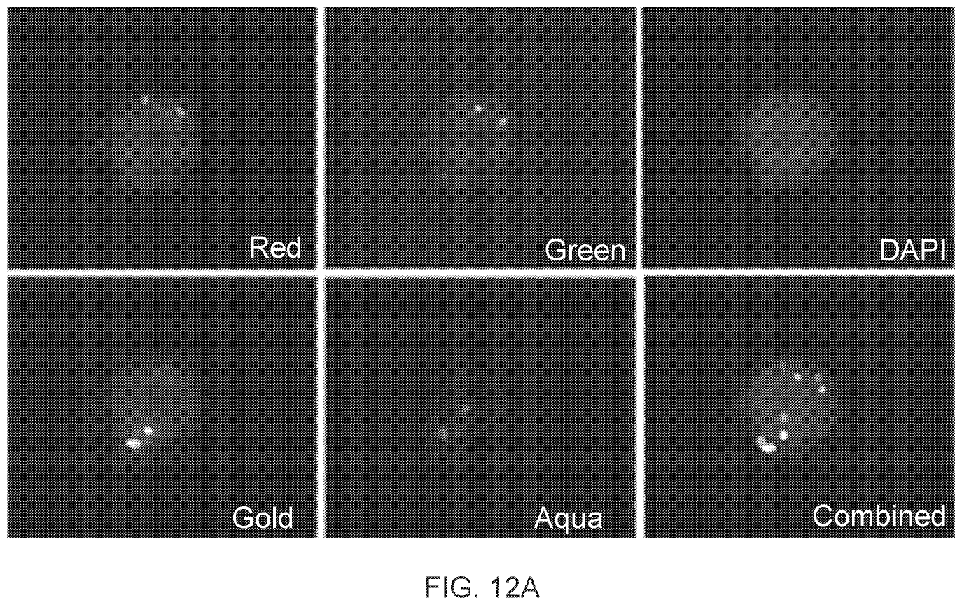

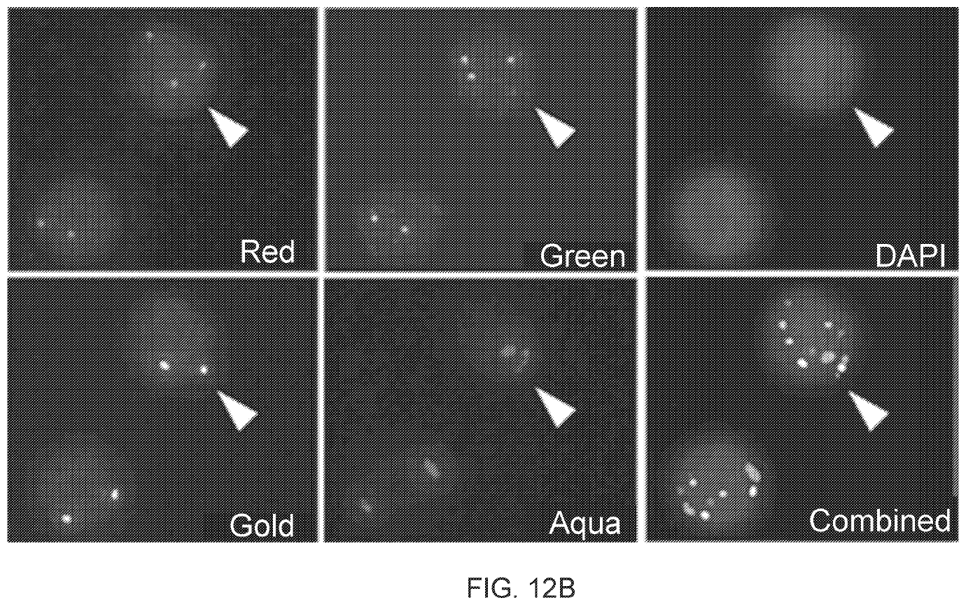

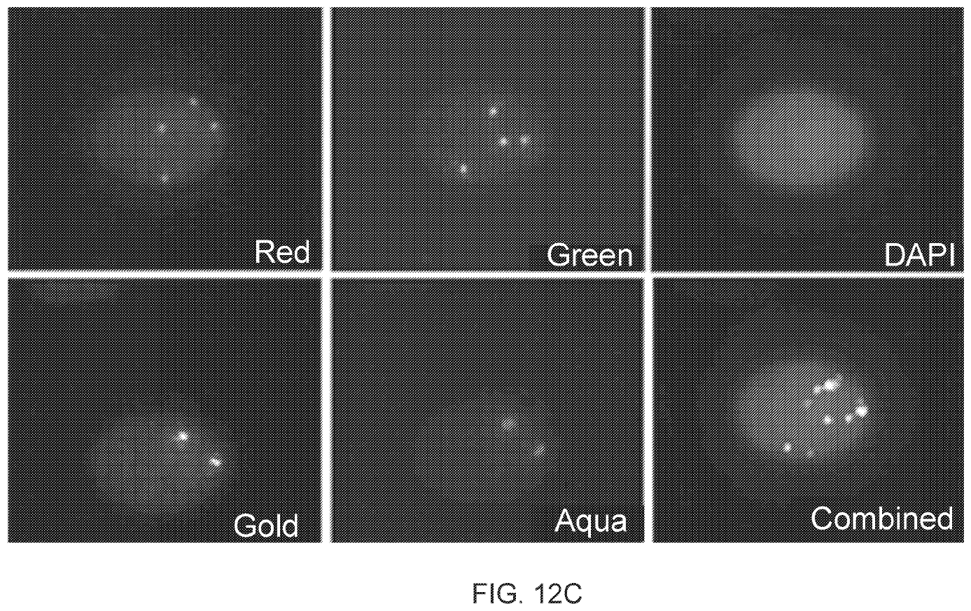

The LungLB™ test utilizes a 4-color FISH assay to detect CNVs in cells from peripheral blood and identify CGACs. A representative test result of a normal white blood cell (WBC) with a diploid copy number per FISH probe, indicated by 2 spots detected per probe color channel (Red, Green, Aqua, and Gold), is presented in FIG. 12A. The LungLB™ test identifies CGACs based a gain of spots in ≥2 color channels. Although there existed cells with spot abnormalities in a single color channel, only cells with a gain in spots in ≥2 color channels were classified as CGACs. A representative CGAC with an extra spot in the Red and Green channels is shown in FIG. 12B. Two additional CGAC subtypes of interest were identified and were classified as Advanced CGACs. Advanced CGACs had either 4 spots in 2 color channels (4×2 Advanced CGAC) as shown in FIG. 12C, or a gain of spots in 2 color channels plus any loss of spots in 2 color channels (Double Deletion Advanced CGAC) as shown in FIG. 12D. Though Advanced CGACs are a subtype of CGACs, classification of a cell as a CGAC or Advanced CGAC was mutually exclusive.

### CGAC Characterization

In total, the LungLB™ test detected 1592 CGACs or Advanced CGACs in the 151 participants who met all study inclusion criteria (Table 14); 1528 (95.8%) were classified as CGACs and 64 (4.0%) were classified as Advanced CGACs. Out of the 151 participants, 148 (98.0%) participant samples contained CGACs and 48 (31.8%) participant samples contained Advanced CGACs. CGACs were detected in all 112 (100%) samples from participants with confirmed malignant nodules vs 35 (89.7%) samples from participants with confirmed benign nodules. Furthermore, Advanced CGACs were detected in 44 (39.3%) samples from participants with confirmed malignant nodules vs 4 (10.3%) samples from participants with confirmed benign nodules. There was also a greater number of CGACs (P=0.004) and Advanced CGACs (P=0.001) identified among malignant versus benign participant samples.

TABLE 14
CGAC and Advanced CGAC Cell Counts in Malignant
and Benign Participant Samples

| | | | | | P value |
|---|---|---|---|---|---|
| | | | Malignant | Benign | (Malignant vs |
| | | Participant | Participant | Participant | Benign |
| | Total Cell | Samples With | Samples with | Samples with | Participant |
| | Count | CGAC | CGAC | CGAC | Samples with |
| n (%) | (n = 1592) | (n = 151) | (n = 112) | (n = 39) | CGAC) |
| CGAC | 1528 (95.8) | 148 (98.0) | 112 (100) | 35 (89.7) | .004^{a} |
| Advanced | 64 (4.0) | 48 (31.8) | 44 (39.3) | 4 (10.3) | .001^{b} |
| CGAC | | | | | |

Abbreviations: CGAC, circulating genetically abnormal cell.
^{a}Fisher's Exact t-test.
^{b}Chi-Square Test.

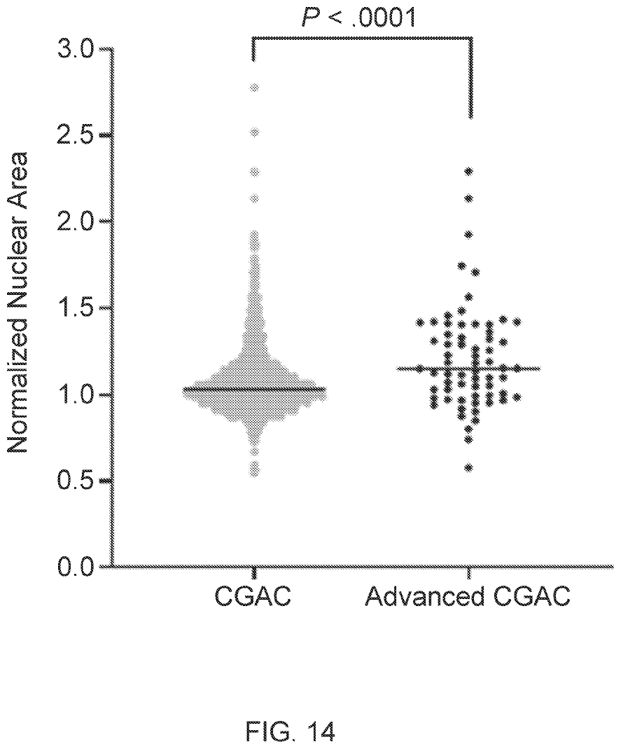

To further characterize CGACs and Advanced CGACs, a morphological analysis was performed to determine their nuclear areas and normalized them to the average nuclear area of each respective participant's normal WBCs. Although CGACs exhibited highly variable nuclear areas, it was observed that the mean nuclear area of Advanced CGACs was approximately 21.1% larger than the average WBC and approximately 13% larger than CGACs (P<0.0001) (FIG. 14 and Table 15).

TABLE 15
Normalized Nuclear Area of CGACs and Advanced
CGACs From All Participant Samples

| Cate- | | Median | Mean | | | P |
|---|---|---|---|---|---|---|
| gory | n | (IQR) | (95% CI) | SD | SEM | value |
| CGAC | 1528 | 1.032 | 1.072 | 0.185 | 0.004 | <.0001 |
| | | (0.547-1.130) | (1.063-1.080) | | | |
| Ad- | 68 | 1.152 | 1.211 | 0.302 | 0.038 | |
| vanced | | (0.995-1.385) | (1.136-1.286) | | | |
| CGAC | | | | | | |

Abbreviations: CGAC, circulating genetically abnormal cell; CI, confidence interval; IQR, interquartile range; SD, standard deviation; SEM, standard error of the mean; WBC, white blood cell.

The normalized nuclear area of every CGAC (gray dots) and Advanced CGAC (black dots) is shown above. The nuclear areas of CGACs and Advanced CGACs were normalized to the average nuclear area of normal WBCs from each respective participant sample. Median (IQR) and mean (95% CI, SD, SEM) normalized nuclear area of CGACs and Advanced CGACs are presented. Normalized nuclear area values >1 indicate a larger nuclear area compared with normal WBCs; normalized nuclear area values <1 indicate smaller nuclear area compared with normal WBCs. The normalized nuclear area of Advanced CGACs was significantly larger than CGACs (P<0.0001).

### LungLB™ Test Performance

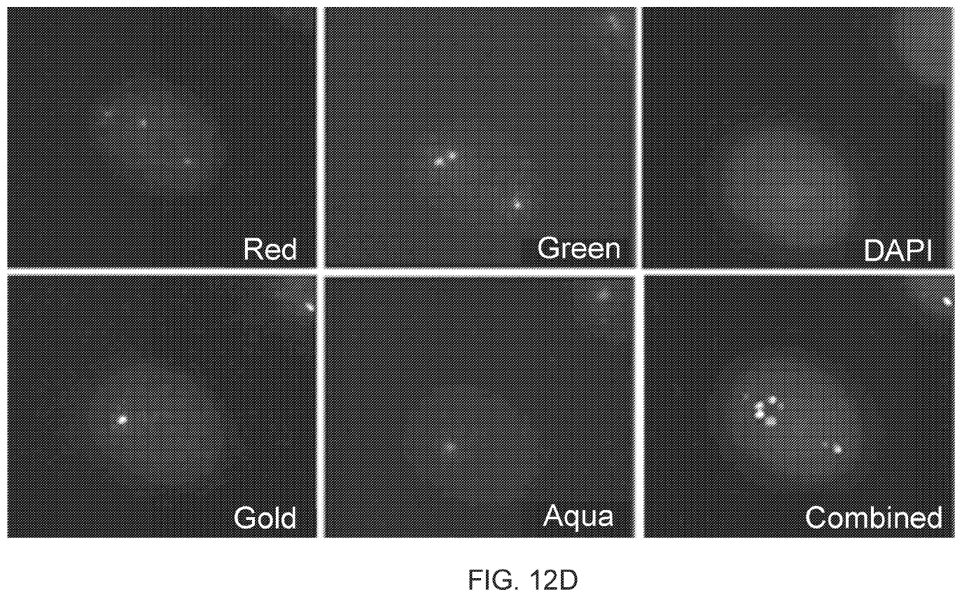

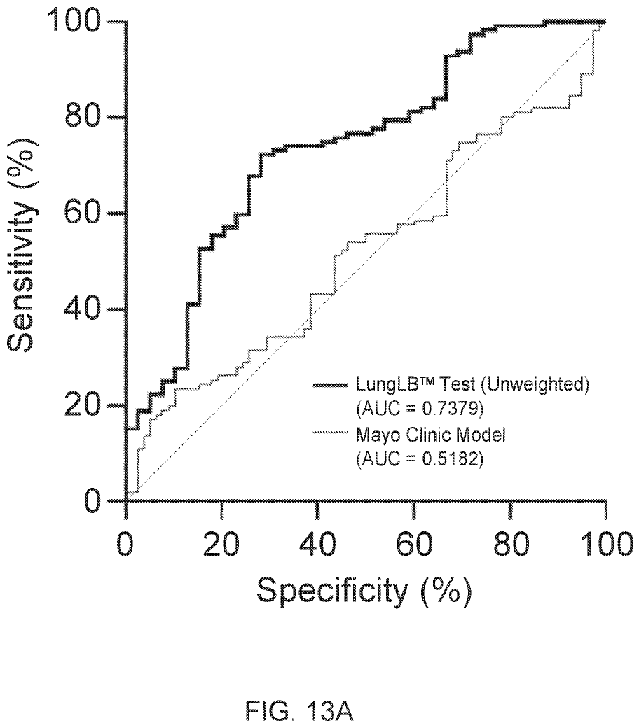

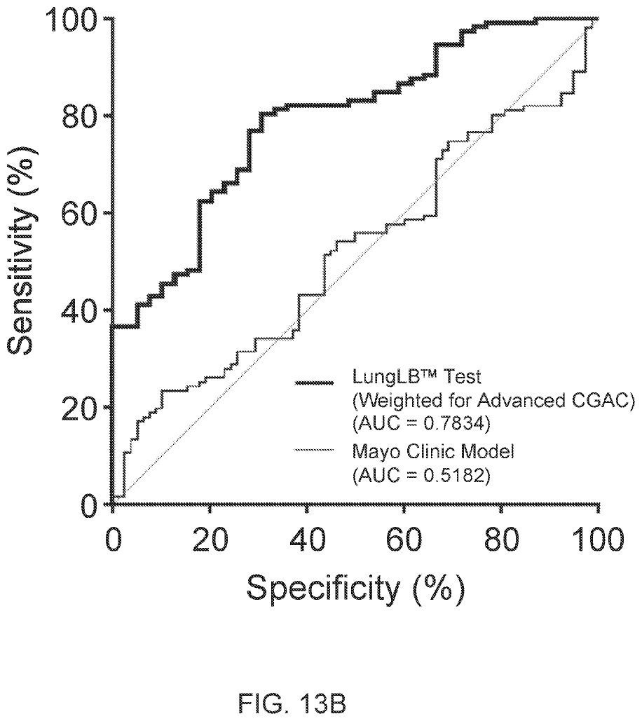

The overall performance and diagnostic efficacy of the LungLB™ test was evaluated by plotting ROC curves in comparison to the ROC from the Mayo Clinic Model. ROC analysis of the full data set consisting of 151 participants revealed an AUC of 0.74 (95% CI, 0.66-0.84; P<0.001), compared with an AUC of 0.52 using the Mayo Clinic Model, and 67.9% sensitivity and 74.4% specificity (FIG. 13A, Table 16). Given the observation that Advanced CGACs were more highly associated with malignant lung cancer diagnosis, an additional ROC analysis where Advanced CGACs were weighted more heavily revealed an AUC of 0.78 (95% CI, 0.70-0.87; P<0.0001), compared with an AUC of 0.52 using the Mayo Clinic Model, and 77% sensitivity and 72% (FIG. 13B, Table 3).

TABLE 16
LungLB ™ Test Performance by Participant Characteristic

| | | | Sensitivity | Specificity |
|---|---|---|---|---|
| Category | n | AUC | (%) | (%) |
| All Samples (Mayo | 151 | 0.5182 | 26.1 | 76.9 |
| Clinic Model) | | | | |
| All Samples (Unweighted) | 151 | 0.7379 | 67.9 | 74.4 |
| All Samples (Weighted | 151 | 0.7834 | 76.8 | 71.8 |
| for Advanced CGACs) | | | | |
| Nodule Size | | | | |
| <2 cm | 83 | 0.8269 | 83.9 | 69.5 |
| ≥2 cm | 68 | 0.7049 | 69.1 | 69.2 |
| Nodule Consistency | | | | |
| Solid | 92 | 0.7215 | 65.2 | 65.2 |
| Sub/Nonsolid | 41 | 0.8952 | 83.3 | 80.0 |
| Cancer Stage | | | | |
| I | 67 | 0.8164 | 74.2 | 74.4 |
| II-IV | 21 | 0.7179 | 61.1 | 71.7 |
| Unknown | 24 | 0.7086 | 65.6 | 78.1 |
| Malignant Lesion Type | | | | |
| Adenocarcinoma | 73 | 0.7875 | 72.6 | 74.4 |
| Squamous Cell Carcinoma | 17 | 0.8250 | 88.2 | 71.8 |
| Small Cell and | 17 | 0.7541 | 76.5 | 71.8 |
| Neuroendocrine | | | | |
| Other | 5 | 0.6970 | 60.0 | 69.2 |
| Smoking History | | | | |
| Ever | 120 | 0.7929 | 76.4 | 71.0 |
| Never | 30 | 0.8075 | 78.3 | 85.7 |

Abbreviations: AUC, area under the curve, CGAC, circulating genetically abnormal cell.

Diagnostic efficacy of the LungLB™ test was also evaluated for various participant characteristics, including nodule size and consistency, lung cancer subtype and stage, and smoking history (Table 16). The LungLB™ test demonstrated robust performance for each participant characteristic category. Superior LungLB™ test performance was observed with nodules <2 cm in size (AUC=0.83) vs ≥2 cm in size (AUC=0.70), sub/nonsolid nodules (AUC=0.90) vs solid nodules (AUC=0.72), and in stage I (AUC=0.82) vs stage II-IV disease (AUC=0.72). Comparable test performance was observed between lung cancer subtypes (adenocarcinoma, AUC=0.79; squamous cell carcinoma, AUC=0.83; and small cell and neuroendocrine, AUC=0.75) and presence of smoking history (Ever, AUC=0.79; and Never, AUC=0.81).

A multivariate analyses was also performed to identify independent predictors of lung cancer and to determine whether inclusion of clinical factors (eg, age, sex, smoking history, presence of COPD/emphysema, nodule type, location and size, and cancer history) in the LungLB™ test may improve overall test performance. Seven different models in the multivariate analyses were compared: 1. CGAC Unweighted (AUC=0.74), 2. CGAC Weighted (AUC=0.78), 3. Clinical Factors (AUC=0.67), 4. CGAC Unweighted+Clinical Factors (AUC=0.74), 5. CGAC Weighted+Clinical Factors (AUC=0.78), 6. CGAC Unweighted+Nodule Size (AUC=0.76), and 7. CGAC Weighted+Nodule Size (AUC=0.78) (Table 17). Although there were no statistically significant differences when the AUCs across the different models was compared, comparison of Model 3 vs Model 5 revealed a P=0.053, which suggests that combining the weighted presence of Advanced CGAC to clinical factors improves the diagnostic efficacy of the LungLB™ test compared with when clinical factors are used alone (Table 18).

TABLE 17
Multivariate Analyses to Identify Independent Predictors
of Malignancy in the LungLB ™ Test

| | | OR | P- |
|---|---|---|---|
| | AUC | (95% CI) | value |
| Model 1 | | | |
| CGAC (Unweighted) | 0.74 | 1.81 (1.35-2.45) | <.001 |
| Model 2 | | | |
| CGAC (Weighted for | 0.78 | 1.44 (1.20-1.72) | <.001 |
| Advanced CGACs) | | | |
| Model 3 | | | |
| Clinical Factors | 0.67 | | |
| Age | | 1.00 (0.94-1.06) | .97 |
| Male | | 1.45 (0.54-3.89) | .46 |
| Ever Smoke | | 0.77 (0.18-3.20) | .72 |
| COPD/Emphysema | | 0.45 (0.16-1.26) | .13 |
| Subsolid/Nonsolid Nodule | | 3.08 (0.91-10.43) | .07 |
| Upper Nodule Location | | 0.80 (0.29-2.19) | .66 |
| Nodule Size | | 1.40 (0.85-2.33) | .19 |
| Cancer History | | 1.01 (0.34-3.00) | .99 |
| Model 4 | | | |
| CGAC (Unweighted) + | 0.74 | | |
| Clinical Factors | | | |
| CGAC (Unweighted) | | 1.65 (1.14-2.38) | .01 |
| Age | | 0.98 (0.92-1.04) | .53 |
| Male | | 1.57 (0.56-4.41) | .39 |
| Ever Smoke | | 0.92 (0.20-4.16) | .91 |
| COPD/Emphysema | | 0.52 (0.18-1.53) | .24 |
| Subsolid/Nonsolid Nodule | | 3.20 (0.85-12.02) | .09 |
| Upper Nodule Location | | 0.66 (0.22-1.97) | .46 |
| Nodule Size | | 1.56 (0.91-2.65) | .10 |
| Cancer History | | 0.98 (0.31-3.16) | .98 |
| Model 5 | | | |
| CGAC (Weighted for Advanced | 0.78 | | |
| CGACs) + Clinical Factors | | | |
| CGAC (Weighted for | | 1.36 (1.11-1.66) | .00 |
| Advanced CGAC) | | | |
| Age | | 0.99 (0.93-1.06) | .84 |
| Male | | 1.57 (0.54-4.51) | .41 |
| Ever Smoke | | 0.53 (0.11-2.47) | .41 |
| COPD/Emphysema | | 0.60 (0.20-1.83) | .37 |
| Subsolid/Nonsolid Nodule | | 3.44 (0.93-12.66) | .06 |
| Upper Nodule Location | | 0.88 (0.30-2.61) | .82 |
| Nodule Size | | 1.64 (0.95-2.84) | .08 |
| Cancer History | | 1.09 (0.34-3.50) | .89 |
| Model 6 | | | |
| CGAC (Unweighted) + | 0.76 | | |
| Nodule Size | | | |
| CGAC (Unweighted) | | 1.78 (1.31-2.43) | <.001 |
| Nodule Size | | 1.47 (1.00-2.18) | .05 |
| Model 7 | | | |
| CGAC (Weighted for Advanced | 0.78 | | |
| CGACs) + Nodule Size | | | |
| CGAC (Weighted for | | 1.42 (1.19-1.71) | <.001 |
| Advanced CGACs) | | | |
| Nodule Size | | 1.48 (0.99-2.21) | .05 |

Abbreviations: AUC, area under the curve; CI, confidence interval; COPD, chronic obstructive pulmonary disease; OR, odds ratio.

Multivariate analyses were performed to determine independent predictors of lung cancer. Seven different models were established to identify independent predictors of lung cancer and to determine whether different combination of these variables may improve overall LungLB™ test performance. The independent predictors that were evaluated included CGAC (Model 1), CGAC with weighting of Advanced CGACs (Model 2), Clinical Factors (Model 3), CGAC+Clinical Factors (Model 4), CGAC with weighting of Advanced CGACs+Clinical Factors (Model 5), CGAC+Nodule Size (Model 6), and CGAC with weighting of Advanced CGACs+Nodules Size (Model 7).

TABLE 18
DeLong Comparison of the Multivariate Models Evaluating Independent
Predictors of Malignancy in the LungLB ™ Test

| Models Comparison | P-value |
|---|---|
| 1 vs 2 | .208 |
| 1 vs 3 | .270 |
| 1 vs 4 | .949 |
| 1 vs 5 | .672 |
| 1 vs 6 | .911 |
| 1 vs 7 | .598 |
| 2 vs 3 | .104 |
| 2 vs 4 | .639 |
| 2 vs 5 | .903 |
| 2 vs 6 | .657 |
| 2 vs 7 | .977 |
| 3 vs 4 | .106 |
| 3 vs 5 | .053 |
| 3 vs 6 | .233 |
| 3 vs 7 | .115 |
| 4 vs 5 | .567 |
| 4 vs 6 | .966 |
| 4 vs 7 | .664 |
| 5 vs 6 | .758 |
| 5 vs 7 | .927 |
| 6 vs 7 | .401 |

Abbreviations: AUC, area under the curve.

The DeLong test was used to compare the AUCs between various multivariate models presented in Table 18. No statistically significant differences were identified between multivariate models, but comparison of Models 3 vs 5 demonstrated a P=0.053.

### Discussion

For individuals with IPNs, various clinical and radiologic factors have been found to be associated with higher risk of lung cancer (Paez R et al, 2021). The Mayo Clinic developed a highly validated and commonly used model that relies on an individual's age, smoking status, history of cancer, and nodule characteristics and location to predict lung cancer risk in individuals with suspicious lung lesions. However, assessment of these factors alone has proved insufficient for high confidence risk stratification of individuals with IPNs and there exists a need for additional independent predictors and biomarkers of lung cancer.¹⁰ When the Mayo Clinic Model was applied to characterize the participants in the study described herein, 15.9% of participants had high risk and 13.2% had low risk of lung cancer. The majority (70.9%) of participants fell into the intermediate-risk class, for which risk stratification in clinical practice remains challenging. This class would also derive the greatest benefit from improved lung cancer prediction tests evaluating novel biomarkers, such as the LungLB™ test, that can better inform clinical decision-making.

In this study to evaluate the performance of the LungLB™ test, multivariate analysis revealed that CGACs were the strongest independent predictor of malignant lung cancer and improved predictive power was achieved when used in adjunct with clinical factors. The performance of the LungLB™ on this participant population was also found to be superior to that of the Mayo Clinic Model. This finding suggests that CGACs may serve as a useful biomarker, and integrating CGAC count with clinical factors in lung cancer risk assessments may provide superior results with greater confidence (Paez R et al, 2021).

The diagnostic value of Advanced CGACs was also found to be greater than other CGACs in detecting malignant lung cancer. Interestingly, it was discovered that Advanced CGACs displayed a larger average nuclear area compared with normal WBCs and other CGACs. Abnormal nuclear morphologies, including an enlarged nucleus, have been previously reported in malignant cells, which support the finding that Advanced CGACs are more highly correlated with malignancy in the LungLB™ test (Fischer E G et al, 2020; Zhou J et al, 2019, Gao W et al, 2017).³⁸

This discovery has additional implications for other diagnostic tests that detect CTCs. CTC enrichment commonly relies on isolation based on cell surface markers and size exclusion (Miller M C et al, 2010. Zhang Z et al, 2015, Habli Z et al, 2020, Rijavec E et al, 2020). Therefore, CGACs without expression of traditional CTC surface markers or that fall outside of the isolation size window would be excluded in these tests. Because the LungLB™ test utilizes FISH to identify CGACs, this test allows for more unbiased detection of CGACs regardless of their surface marker expression profile or size.

The instant analyses revealed that the LungLB™ test displayed consistent and robust performance across various participant characteristics. Surprisingly, the LungLB™ test continued to display robust performance even in participants with smaller (<2 cm) nodules, sub/nonsolid nodules, and stage I disease. The finding that the LungLB™ test performed well in participants with stage I disease was surprising, given that other cancer detection tests typically exhibit poor performance with detecting early-stage disease (Klein E A et al, 2021, Freitas C et al, 2021). The LungLB™ test demonstrated 74.2% sensitivity in stage I lung cancer and 61.1% sensitivity across stage II-IV lung cancer. This finding is particularly impactful given that early-stage lung cancer is where there currently exists the greatest unmet need in terms of an accurate, minimally invasive diagnostic test.

Furthermore, ROC analysis revealed that the LungLB™ test performed with 71.8% specificity. Hypothetically, if the LungLB™ test results had been available and utilized in the clinical decision-making for participants with benign lesions in this study, 29 of the 39 participants may have been spared from undergoing an invasive biopsy (39 participants with benign lesions×71.8% specificity=29 participants). In addition to sparing individuals with IPNs from unnecessary biopsies, the LungLB™ test also may offer earlier detection of lung cancer compared with current standard of care procedures and practices. In the example cases presented, initial biopsies indicated a negative diagnosis in Case 1 and an indeterminant diagnosis in Case 2. Meanwhile, the LungLB™ test, which was performed concurrently with the biopsies, positively detected lung cancer in both cases. Later analysis of surgically resected tissue revealed that both patients had lung cancer, which was misdiagnosed by the initial biopsy but correctly predicted by the LungLB™ test. Notably in Case 2, surgical resection of the nodule did not occur until 2 years after the initial biopsy misdiagnosis. Although the volume doubling times of IPNs are highly variable, malignant lung cancer nodules are associated with rapid doubling times and aggressive metastasis; therefore, delays in proper diagnosis and treatment of malignant lung cancer have significant implications and may lead to poorer patient outcomes (Heiden B T et al, 2021). Additionally, the inability to provide accurate diagnoses with initial biopsies also subjects patients to the burden of frequent follow-up screenings and examinations and continued anxiety, especially if the biopsy procedure resulted in an adverse event. Altogether, this highlights the broad potential clinical utility of the LungLB™ test.

This study is not without limitations; all participant samples were collected from 2 sites and comprise a relatively limited sample set, which do not span the full diversity of patient demographics nor IPNs that exist. The multivariate models were developed using this small dataset; therefore, there is risk of overfitting in these models, which will be addressed in future larger-scale studies. Of importance to note is that one of the sites participants were enrolled from was the MD Anderson Cancer Center in Houston, Texas, which is in a region with higher incidence of fungal histoplasmosis infections. For clinicians, distinguishing infectious etiology from lung cancer is often challenging and presence of histoplasmosis infection can confound lung cancer diagnosis (Grogan et al, 2014). In the instant study, 11 (28.2%) of 39 participants with benign lesions had infectious etiology. The LungLB™ test maintained strong performance in these individuals with confounding lung infections, further demonstrating its promise in diagnosing lung cancer in challenging populations.

The LungLB™ test utilizes FISH, a highly specific and sensitive assay, to detect DNA CNVs that are a hallmark of cancer to broadly detect CGACs. However, because the test does not rely on the traditional markers of CTCs to identify CGACs, further characterization of CGACs is warranted and immunophenotyping of these cell populations is underway. Further characterization of these cells may help in understanding the pathogenesis of lung cancer, as well as provide the potential for identification of additional cell markers that can be used to further improve the performance of the LungLB™ test. Additionally, novel biomarker targets may also be revealed, which can be leveraged in the development of targeted therapies.

The presented data indicate that the LungLB™ test, a liquid biopsy, FISH-based assay, is capable of discriminating benign from malignant processes in individuals with IPNs at risk for lung cancer. It is reported that the LungLB™ test performs with high specificity and sensitivity, which is further enhanced when combined with clinical factors. Multisite validation studies are underway to validate the clinical performance of the LungLB™ test in the assessment of IPNs in a broader patient population.

### Example 6: Combination of CGAC Chromosomal Hybridization Pattern and One or More Nuclear Parameters

A study was conducted to evaluate which characteristics (probe patterns and cell morphology) correlate more with malignancy and how much weight should be assigned to distinct nuclear parameters and the specific chromosomal hybridization pattern.

A series of univariate analysis were performed to determine what parameters were predictive of malignancy in a sample obtained from a subject. The analysis was performed on double deletion cells and the impact of nuclear area, nuclear roundness, and nuclear diameter was assessed to determine which combination of factors gave the strongest predictor of malignancy.

A Copy Number Variation Score was calculated for the chromosomal hybridization pattern and the CNV score was scaled according to the normalized and weighted nuclear area, nuclear roundness, and nuclear diameter to generate a CGAC score. Normalized Nuclear Area, Roundness, and Diameter are generated by normalizing these parameters to the normal cells on that patients slide.

An area under the curve (AUC) analysis was performed for each analysis in FIGS. 15A-15D. For the double deletion analysis in FIG. 15A, the AUC was 0.64. For the analysis in FIG. 15B which further factored normalized nuclear area, the AUC slightly improved to 0.65. For the analysis in FIG. 15C which further factored normalized nuclear roundness, the AUC did not improve. For the analysis in FIG. 15D, which further factored normalized nuclear area, normalized nuclear diameter, and weighted normalized roundness the AUC improved to 0.71 demonstrating an ability to differentiate between benign and malignant cells. In this example, roundness was assigned a weighted value to increase its contribution to the method. Raw roundness values ranged from 1.1 to 1.5 range. However the average normalized roundness for benign versus malignant was 1.1 vs 1.2. In order to add more weight to the Roundness parameter 1.0 was subtracted from the raw output so that the new weighted outputs would be 0.100 vs 0.200. This created a 2× difference rather than the less than 10% difference observed originally. This weighting was performed to determine if such weighting could further stratify benign versus malignant samples.

For the double deletion analysis in FIG. 15A, the AUC was 0.64. For the analysis in FIG. 15B which further factored normalized nuclear area, the AUC slightly improved to 0.65. For the analysis in FIG. 15C which further factored normalized nuclear roundness, the AUC did not improve. For the analysis in FIG. 15D, which further factored normalized nuclear area, normalized nuclear diameter, and weighted normalized roundness the AUC improved to 0.71 demonstrating an ability to differentiate between benign and malignant cells

### CGAC Score Calculation

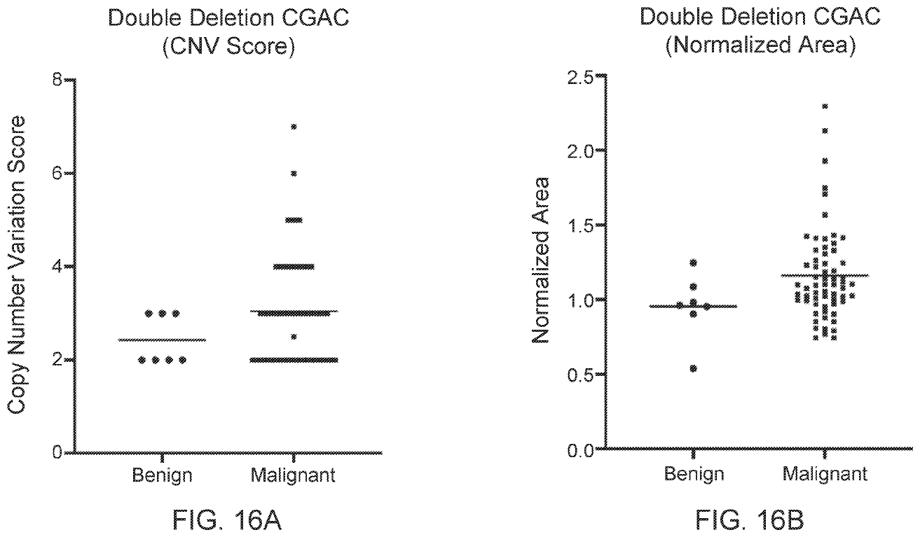

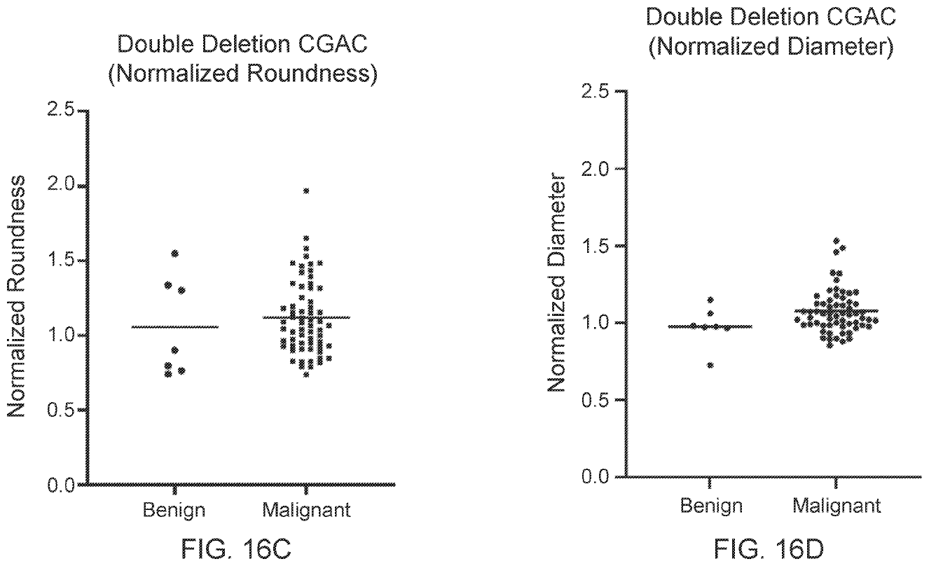

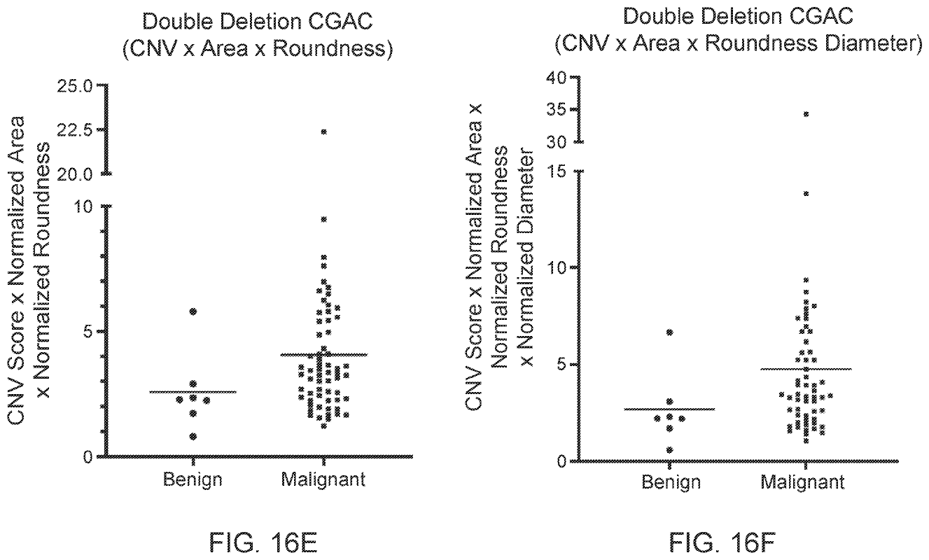

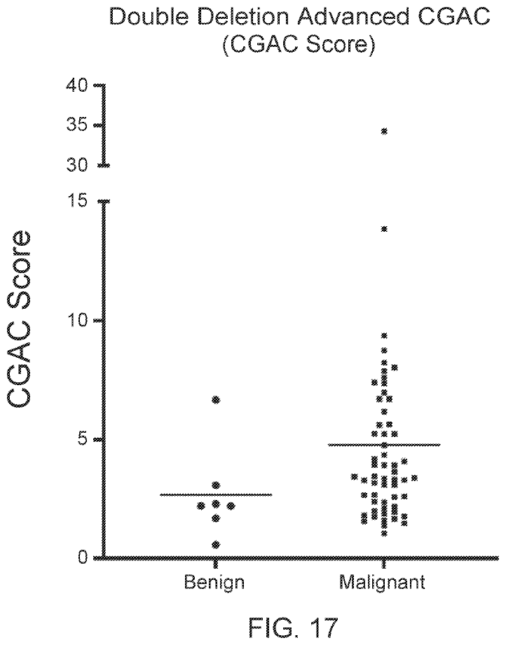

An area under the curve (AUC) analysis was performed for each analysis in FIGS. 16A-16E. The AUC values for each are detailed in table 19. A CGAC score calculated that factors CNV score, normalized nuclear area, normalized nuclear diameter, and normalized nuclear roundness displayed greater diagnostic power that an analysis using a single variable and CNV score or CNV score alone. A CGAC score calculated using this combination of features shows an enhanced ability to differentiate between benign and malignant double deletion CGACs.

TABLE 19
CGAC Score AUC analysis Summary

| CGAC Score | Number of | | |
|---|---|---|---|
| Model | Parameters | AUC | p-value |
| CNV Score | 1 | 64.91% | 0.2006 |
| Normalized Area | 1 | 69.67% | 0.0913 |
| Normalized | 1 | 60.90% | 0.3494 |
| Roundness | | | |
| Normalized | 1 | 70.15% | 0.0840 |
| Diameter | | | |
| CNV Score × Area | 2 | 71.93% | 0.0598 |
| CNV Score × | 2 | 68.17% | 0.1189 |
| Roundness | | | |
| CNV score × Area × | 3 | 70.93% | 0.0725 |
| Roundness | | | |
| CNV Score × Area × | 4 | 71.93% | 0.0598 |
| Roundness × Diameter | | | |

### Example 7: Exemplary CGAC Score Calculation

A CGAC identified in a patient sample was evaluated according to its nuclear hybridization pattern which are set forth in Table 20. For the cell taken from the malignant sample, the CGAC has a Nuclear Area, Roundness, and Diameter that are all higher than the baselines established from its biological sample. Therefore, the CGAC's Normalized Nuclear Parameters are all >1.0 and this CGAC will be weighted higher. For the cell taken from the benign sample, the CGAC has a Nuclear Area, Roundness, and Diameter that are all lower than the baselines established from its biological sample. Therefore, the CGAC's Normalized Nuclear Parameters are all <1.0 and this CGAC will be weighted lower.

TABLE 20
CGAC values for exemplary CGAC

| Sample Diagnosis | Malignant | Benign |
|---|---|---|
| CNV Score | 2 | 3 |
| CGAC Area | 153.772 | 117.188 |
| CGAC Roundness | 175 | 118 |
| CGAC Diameter | 14.385 | 12.3 |
| Sample Mean Area | 108.854 | 123.038 |
| Sample Mean Roundness | 151.844 | 148.017 |
| Sample Mean Diameter | 11.958 | 12.729 |
| CGAC Normalized Area | 1.413 | 0.952 |
| CGAC Normalized | 1.152 | 0.797 |
| Roundness | | |
| CGAC Normalized | 1.203 | 0.966 |
| Diameter | | |
| CGAC Score | 3.917 | 2.201 |

For the sample, the CGAC score was calculated according to the following equation:

$$CGAC Score = CNV Score \times Normalized Area \times Normalized Roundness \times Normalized Diameter .$$

The CGAC Score algorithm aims to rank CGAC as more or less likely to be malignant by considering all factors such as the CNV Score, Normalized Nuclear Area, Normalized Nuclear Roundness, and Normalized Nuclear Diameter. An initial analysis, specifically focused on Double Deletion Advanced CGAC, reveals that the combination of all parameters provides the most holistic approach and the best results. Small/Round CGAC are penalized while Large/Oval CGAC with abnormal morphology are weighted more heavily. This example demonstrates how CNV Score alone is insufficient to delineate a Malignant Double Deletion (CNV Score=2) compared to a Benign Double Deletion (CNV Score=3). It is only when CNV Score AND Nuclear Parameters are used together that the Malignant Double Deletion (CGAC Score=3.917) is correctly separated from the Benign Double Deletion (CGAC Score=2.201).

### Advanced CGAC Type CGAC Score Calculation

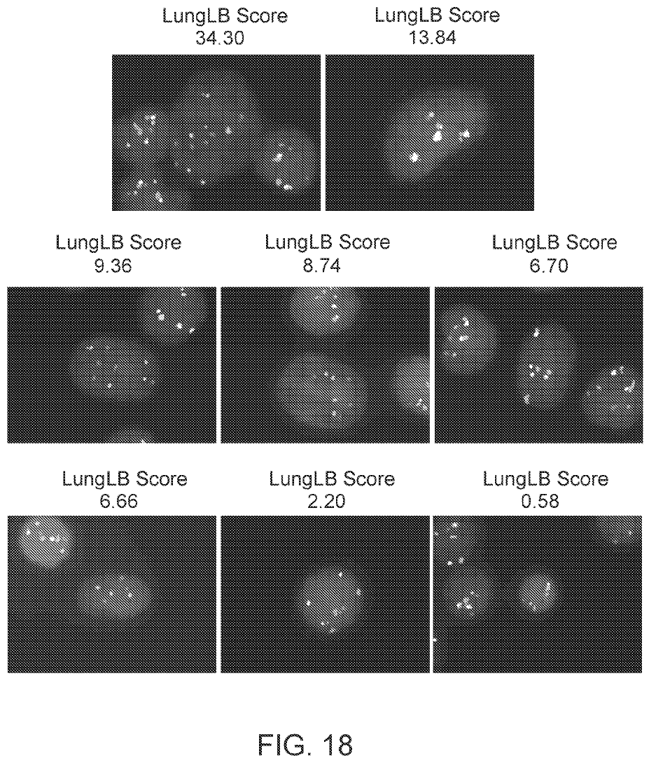

The CGAC score for CGACs having advanced CGAC hybridization patterns was calculated and plotted in FIG. 17. A significant difference in CGAC score is depicted between benign and malignant advanced CGAC types. FIG. 18 is a series of photographs demonstrating that the CGAC score for a given CGAC can be quite high when a large number of chromosomal duplications are present and the nuclear area increased, the nuclear diameter increases, and the nuclear roundness becomes less round.

1. A method for identifying lung cancer in a subject in need thereof comprising:
(a) contacting a biological sample obtained from the subject comprising a population of cells with fluorescently labeled nucleic acid probes and performing fluorescence in situ hybridization to determine chromosomal hybridization patterns;
(b) identifying circulating genetically abnormal cells (CGAC) in the population of cells based on the chromosomal hybridization patterns determined in step (a);
(c) for each CGAC identified in step (a) performing at least one of:
(i) determining the presence or absence of at least one intracellular or cell surface protein;
(ii) determining at least one nuclear parameter selected from nuclear area, nuclear roundness, nuclear diameter, or a combination thereof, and
(d) determining a CGAC score for each CGAC based on the chromosomal hybridization pattern and at least one of:
(i) the presence or absence of the intracellular or cell surface protein; or
(ii) the at least one nuclear parameter;
(e) determining a sample score by taking the sum of the CGAC scores for all CGAC identified in the biological sample obtained from the subject; and
(f) identifying lung cancer in the subject when the sample score is above a predetermined cutoff value.

2. The method of claim 1, wherein the fluorescently labeled nucleic acid probes comprise four pluralities of nucleic acid probes, wherein each plurality of nucleic acid probes hybridizes to a distinct chromosomal sequence and comprises a distinct fluorescent label, wherein a first plurality of fluorescently labeled nucleic acid probes hybridizes to 3p22.1, a second plurality of fluorescently labeled nucleic acid probes hybridizes to 10q22.3, a third plurality of fluorescently labeled nucleic acid probes hybridizes to chromosome 10 centromeric (cep10), and a fourth plurality of fluorescently labeled nucleic acid probes hybridizes to 3q29.

3.-5. (canceled)

6. The method of claim 1, wherein a CGAC comprises:
a chromosomal hybridization pattern that comprises: a gain of at least one copy of a first chromosomal sequence, a gain of at least one copy of a second chromosomal sequence, a loss of at least one copy of a third chromosomal sequence, and a loss of at least one copy of a fourth chromosomal sequence;
a chromosomal hybridization pattern that comprises five or more copies of a first chromosomal sequence and five or more copies of a second chromosomal sequence; and/or
a chromosomal hybridization pattern that comprises: at least four copies of a first chromosomal sequence, at least four copies of a second chromosomal sequence, two copies of a third chromosomal sequence, two copies of a fourth chromosomal sequence.

7.-10. (canceled)

11. The method of claim 1, wherein a CGAC comprises: at least three copies of a first chromosomal sequence, at least three copies of a second chromosomal sequence, at least three copies of a third chromosomal sequence, and at least three copies of a fourth chromosomal sequence.

12. (canceled)

13. The method of claim 1, wherein the CGAC score comprises a copy number variation (CNV) score assigned based on the chromosomal hybridization pattern.

14. The method of claim 1, wherein the CNV score for a healthy cell is zero.

15. The method of claim 1, wherein:
the CNV score is increased by a value of 0.5 for each gain of a chromosomal sequence; and/or
the copy number variation score is increased by a value of 0.5 for each loss of a chromosomal sequence.

16. (canceled)

17. The method of claim 1, wherein the CGAC score is determined by scaling the CNV score according to at least one of:
(i) to the presence or absence of the intracellular or cell surface protein; or
(ii) the at least one nuclear parameter,
to determine the CGAC score.

18. The method of claim 1, wherein the nuclear area for a healthy cell identified in the biological sample is assigned a normalized area of about 1.0 and wherein the normalized nuclear area for a CGAC is expressed as a value relative to the nuclear area of the healthy cell.

19.-21. (canceled)

22. The method of claim 1, wherein the CGAC normalized area is multiplied by the CNV score to determine the CGAC score for a CGAC of the biological sample.

23. The method of claim 1, wherein the nuclear roundness for a healthy cell is assigned a normalized roundness of about 1.0 and the normalized nuclear roundness for a CGAC is expressed as a value relative to the normalized roundness of a healthy cell.

24.-26. (canceled)

27. The method of claim 1, wherein the CGAC normalized roundness is multiplied by the CNV score to determine the CGAC score for a CGAC of the biological sample.

28. (canceled)

29. The method of claim 1, wherein a nuclear diameter for a healthy cell is assigned a normalized nuclear diameter of about 1.0 and a normalized nuclear diameter for a CGAC is expressed as a value relative to the normalized diameter of a healthy cell.

30.-32. (canceled)

33. The method of claim 1, wherein the CGAC normalized diameter is multiplied by the CNV score to determine the CGAC score for a CGAC of the biological sample.

34. The method of claim 1, wherein the CNV score can be multiplied by at least one of the CGAC normalized diameter, the CGAC normalized roundness, and the CGAC normalized area, or a combination thereof, to determine the CGAC score for a CGAC of the biological sample.

35. (canceled)

36. (canceled)

37. The method of claim 1, wherein the at least one intracellular or cell surface protein is selected from CD45, CD19, CD31, PAX5, AID, BCL6, EGFR, CD3, CD20, IgM, IgD, CD56, EpCAM, Vimentin, FoxP3, KI-67, or a combination thereof.

38. The method of claim 1, wherein a CGAC is assigned a biomarker score based on the presence or absence of the at least one intracellular or cell surface protein.

39. The method of claim 1, wherein the biomarker score is multiplied by the CNV score to determine the CGAC score for a CGAC of the biological sample.

40. The method of claim 1, wherein the CNV score can be multiplied by at least one of the CGAC normalized diameter, the CGAC normalized roundness, the CGAC normalized area, and the biomarker score or a combination thereof, to determine the CGAC score for a CGAC of the biological sample.

41. The method of claim 1, wherein the population of cells of the biological sample comprises about 5,000 cells, about 6,000 cells, about 7,000 cells, about 8,000 cells, about 9,000 cells, about 10,000 cells, about 11,000 cells, about 12,000 cells, about 13,000 cells, about 14,000 cells, about 15,000 cells, about 16,000 cells, about 17,000 cells, about 18,000 cells, about 19,000 cells, about 20,000 cells, about 50,000 cells, or about 100,000 cells.

42.-44. (canceled)

45. The method of claim 1, wherein the sample score predetermined cutoff value is about 0.5 CGAC/10,000 cells, about 1.0 CGAC/10,000 cells, about 1.5 CGAC/10,000 cells, about 2.0 CGAC/10,000 cells, about 2.5 CGAC/10,000 cells, about 3.0 CGAC/10,000 cells, about 3.5 CGAC/10,000 cells, about 4.0 CGAC/10,000 cells, about 5.0 CGAC/10,000 cells, about 5.5 CGAC/10,000 cells, about 6.0 CGAC/10,000 cells, about 6.5 CGAC/10,000 cells, about 7.0 CGAC/10,000 cells, about 7.5 CGAC/10,000 cells, about 8.0 CGAC/10,000 cells, about 8.5 CGAC/10,000 cells, about 9.0 CGAC/10,000 cells, about 9.5 CGAC/10,000 cells, about 10.0 CGAC/10,000 cells, about 15.0 CGAC/10,000 cells, about 20.0 CGAC/10,000 cells, about 25.0 CGAC/10,000 cells, about 30.0 CGAC/10,000 cells, about 40.0 CGAC/10,000 cells, about 50.0 CGAC/10,000 cells, about 60.0 CGAC/10,000 cells, about 70.0 CGAC/10,000 cells, about 80.0 CGAC/10,000 cells, about 90.0 CGAC/10,000 cells, about 100 CGAC/10,000 cells, about 200 CGAC/10,000 cells, about 300 CGAC/10,000 cells, about 400 CGAC/10,000 cells, about 500 CGAC/10,000 cells, about 600 CGAC/10,000 cells, about 700 CGAC/10,000 cells, about 800 CGAC/10,000 cells, about 900 CGAC/10,000 cells, about 1,000 CGAC/10,000 cells, or about 2,000 CGAC/10,000 cells.

46. The method of claim 1, wherein the biological sample obtained from the subject is a blood sample.

47. The method of claim 1, further comprising performing a CGAC enrichment step prior to contacting the biological sample obtained from the subject comprising:
(i) removing plasma from the sample,
(ii) removing erythrocytes from the sample,
(iii) contacting the sample with at least one affinity agent that binds a cell surface protein, and
(iv) depleting cells from the sample that express the cell surface protein.

48. (canceled)

49. The method of claim 1, further comprising administering a therapeutic agent to the subject having been diagnosed with lung cancer.

50. (canceled)

51. A method for identifying lung cancer in a subject in need thereof comprising:
(a) contacting a biological sample obtained from the subject comprising a population of cells with fluorescently labeled nucleic acid probes and performing fluorescence in situ hybridization to determine chromosomal hybridization patterns;
(b) identifying circulating genetically abnormal cells (CGAC) in the population of cells based on the chromosomal hybridization patterns determined in step (a);
(c) for each CGAC identified in step determining at least one nuclear parameter selected from nuclear area, nuclear roundness, nuclear diameter, or a combination thereof, and
(d) determining a CGAC score for each CGAC based on the chromosomal hybridization pattern and the at least one nuclear parameter;
(e) determining a sample score by taking the sum of the CGAC scores for all CGAC identified in the biological sample obtained from the subject; and
(f) identifying lung cancer in the subject when the sample score is above a predetermined cutoff value.

52. A method for identifying lung cancer in a subject in need thereof comprising:
(a) contacting a biological sample obtained from the subject comprising a population of cells with fluorescently labeled nucleic acid probes and performing fluorescence in situ hybridization to determine chromosomal hybridization patterns;
(b) identifying circulating genetically abnormal cells (CGAC) in the population of cells based on the chromosomal hybridization patterns determined in step (a);
(c) determining a CGAC score for each CGAC based on the chromosomal hybridization pattern;
(d) determining a sample score by taking the sum of the CGAC scores for all CGAC identified in the biological sample obtained from the subject; and
(e) identifying lung cancer in the subject when the sample score is above a predetermined cutoff value.

53. The method of claim 1, further comprising performing a CGAC enrichment step prior to contacting the biological sample obtained from the subject comprising:
(i) removing plasma from the sample,
(ii) removing erythrocytes from the sample,
(iii) contacting the sample with at least one affinity agent that binds a cell surface protein, and
(iv) collecting cells from the sample that express the cell surface protein.
